(19) United States
(12) Patent Application Publication
(10) Pub. No.: US 2006/0063792 A1
(43) Pub. Date: Mar. 23, 2006

(54) Substituted morphinans and methods of their use

(75) Inventors: Roland E. Dolle, King of Prussia, PA (US); Bertrand Le Bourdonnec, East Fallowfield, PA (US); Jonathan Mark Sutton, Chelmsford (GB); Paul Eastwood, Barcelona (ES); Ines Warner, Kelvedon (GB)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Adolor Corporation, Exton, PA (US)

(21) Appl. No.: 11/227,685

(22) Filed: Sep. 15, 2005

Related U.S. Application Data
(60) Provisional application No. 60/610,721, filed on Sep. 17, 2004.

(51) Int. Cl. A61K 31/485; C07D 489/02

(52) U.S. Cl. 514/282; 546/45

(57) ABSTRACT

Novel 4,5-α epoxy-morphinan compounds are disclosed. Pharmaceutical compositions containing the 4,5-α epoxy-morphinan compounds and methods of their pharmaceutical uses are also disclosed. The compounds disclosed are useful, inter alia, as modulators of opioid receptors.

### CROSS REFERENCE TO RELATED APPLICATION

This application claims the benefit of U.S. Provisional Application Ser. No. 60/610,721, filed Sep. 17, 2004, the entire disclosure of which is incorporated herein by reference in its entirety.

### FIELD OF THE INVENTION

The present invention relates to compounds that affect the opioid receptor system and, more particularly, to morphinan compounds and pharmaceutical compositions containing such compounds that are, inter alia, modulators of opioid receptors.

### BACKGROUND OF THE INVENTION

It is well known that opioid drugs target three types of endogenous opioid receptors (i.e., μ, δ, and κ receptors) in biological systems. Many opiates, such as morphine, are μ opioid agonists that are often used as analgesics for the treatment of severe pain due to their activation of μ opioid receptors in the brain and central nervous system (CNS). Opioid receptors are, however, not limited to the CNS, and may be found in other tissues throughout the body, i.e., peripheral to the CNS. A number of side effects of opioid drugs may be caused by activation of these peripheral receptors. For example, administration of μ opioid agonists often results in intestinal dysfunction due to the large number of receptors in the wall of the gut (Wittert, G., Hope, P. and Pyle, D., Biochemical and Biophysical Research Communications, 1996, 218, 877-881; Bagnol, D., Mansour, A., Akil, A. and Watson, S. J., Neuroscience, 1997, 81, 579-591). Specifically, opioids are generally known to cause nausea and vomiting, as well as inhibition of normal propulsive gastrointestinal function in animals and man (Reisine, T., and Pasternak, G., Goodman & Gilman's The Pharmacological Basis of Therapeutics, Ninth Edition, 1996, 521-555), resulting in side effects such as, for example, constipation.

Recent evidence has indicated that naturally-occurring endogenous opioid compounds may also affect propulsive activity in the gastrointestinal (GI) tract. Met-enkephalin, which activates μ and δ receptors in both the brain and gut, is one of several neuropeptides found in the GI tract (Koch, T. R., Carney, J. A., Go, V. L., and Szurszewski, J. H., Digestive Diseases and Sciences, 1991, 36, 712-728). Additionally, receptor knockout techniques have shown that mice lacking μ opioid receptors may have faster GI transit times than wild-type mice, suggesting that endogenous opioid peptides may tonically inhibit GI transit in normal mice (Schuller, A. G. P., King, M., Sherwood, A. C., Pintar, J. E., and Pasternak, G. W., Society of Neuroscience Abstracts 1998, 24, 524). Studies have shown that opioid peptides and receptors located throughout the GI tract may be involved in normal regulation of intestinal motility and mucosal transport of fluids in both animals and man (Reisine, T., and Pasternak, G., Goodman & Gilman's The Pharmacological Basis of Therapeutics, Ninth Edition, 1996, 521-555). Other studies show that the sympathetic nervous system may be associated with endogenous opioids and control of intestinal motility (Bagnol, D., Herbrecht, F., Jule, Y., Jarry, T., and Cupo, A., Regul. Pept., 1993, 47, 259-273). The presence of endogenous opioid compounds associated with the GI tract suggests that an abnormal physiological level of these compounds may lead to bowel dysfunction.

It is a common problem for patients having undergone surgical procedures, especially surgery of the abdomen, to suffer from a particular bowel dysfunction called post-surgical (or post-operative) ileus. “Ileus,” as used herein, refers to the obstruction of the bowel or gut, especially the colon. See, e.g., Dorland's Illustrated Medical Dictionary, 27th ed., page 816, (W.B. Saunders Company, Philadelphia, Pa., 1988). Ileus should be distinguished from constipation, which refers to infrequency of or difficulty in feces evacuation. See, e.g., Dorland's Illustrated Medical Dictionary, 27th ed., page 375, (W. B. Saunders Company, Philadelphia, 1988). Ileus may be diagnosed by the disruption of normal coordinated movements of the gut, resulting in failure of intestinal contents propulsion. See, e.g., Resnick, J., Am. J. of Gastroenterology, 1997, 92, 751 and Resnick, J. Am. J. of Gastroenterology, 1997, 92, 934. In some instances, particularly following surgery, including surgery of the abdomen, the bowel dysfunction may become quite severe, lasting for more than a week and affecting more than one portion of the GI tract. This condition is often referred to as post-surgical (or post-operative) paralytic ileus and most frequently occurs after laparotomy (see Livingston, E. H. and Passaro, Jr., E. D., Digestive Diseases and Sciences, 1990, 35, 121). Similarly, post-partum ileus is a common problem for women in the period following childbirth, and is thought to be caused by similar fluctuations in natural opioid levels as a result of birthing stress.

Gastrointestinal dysmotility associated with post-surgical ileus is generally most severe in the colon and typically lasts for 3 to 5 days. The administration of opioid analgesics to a patient after surgery may often contribute to bowel dysfunction, thereby delaying recovery of normal bowel function. Since virtually all patients receive opioid analgesics, such as morphine or other narcotics, for pain relief after surgery, particularly major surgery, current post-surgical pain treatment may actually slow recovery of normal bowel function, resulting in a delay in hospital discharge and increasing the cost of medical care.

Post-surgical and post-partum ileus may also occur in the absence of exogenous opioid agonists. It would be of benefit to inhibit the natural activity of endogenous opioids during and/or after periods of biological stress, such as surgery and childbirth, so that ileus and related forms of bowel dysfunction can be prevented and/or treated. Currently, therapies for ileus include functional stimulation of the intestinal tract, stool softeners, laxatives, lubricants, intravenous hydration, and nasogastric decompression. These prior art methods suffer from drawbacks, for example, as lacking specificity for post-surgical or post-partum ileus. And these prior art methods offer no means for prevention. If ileus could be prevented, hospital stays, recovery times, and medical costs would be significantly decreased, in addition to the benefit of minimizing patient discomfort. Thus, drugs that selectively act on opioid receptors in the gut would be ideal candidates for preventing and/or treating post-surgical and post-partum ileus. Of those, drugs that do not interfere with the effects of opioid analgesics in the CNS would be of special benefit in that they could be administered simultaneously for pain management with limited side effects.

Peripheral opioid antagonists that do not cross the blood-brain barrier into the CNS are known in the literature and have been tested in relation to their activity on the GI tract. In U.S. Pat. No. 5,250,542, U.S. Pat. No. 5,434,171, U.S. Pat. No. 5,159,081, and U.S. Pat. No. 5,270,328, peripherally selective piperidine-N-alkylcarboxylate opioid antagonists are described as being useful in the treatment of idiopathic constipation, irritable bowel syndrome, and opioid-induced constipation. Some peripheral μ antagonists derived from the structure naltrexone have been reported in the literature (U.S. Pat. No. 4,806,556; Botros, et al., J. Med. Chem. 1989, 32, 2068-2071). In addition, U.S. Pat. No. 4,176,186 describes quaternary derivatives of noroxymorphone (i.e., methylnaltrexone) that are said to prevent or relieve the intestinal immobility side effect of narcotic analgesics without reducing analgesic effectiveness. U.S. Pat. No. 5,972,954 describes the use of methylnaltrexone, enteric-coated methylnaltrexone, or other quaternary derivatives of noroxymorphone for preventing and/or treating opioid- and/or nonopioid-induced side effects associated with opioid administration.

General opioid antagonists, such as naloxone and naltrexone, have also been implicated as being useful in the treatment of GI tract dysmotility. For example, U.S. Pat. No. 4,987,126 and Kreek, M. J. Schaefer, R. A., Hahn, E. F., Fishman, J. Lancet, 1983, 1, 8319, 261 disclose naloxone and other morphinan-based opioid antagonists (i.e., naloxone, naltrexone) for the treatment of idiopathic gastrointestinal dysmotility. In addition, naloxone has been shown to effectively treat non-opioid induced bowel obstruction, implying that the drug may act directly on the GI tract or in the brain (Schang, J. C., Devroede, G., Am. J. Gastroenerol., 1985, 80, 6, 407). Furthermore, it has been implicated that naloxone may provide therapy for paralytic ileus (Mack, D. J. Fulton, J. D., Br. J. Surg., 1989, 76, 10, 1101). However, it is well known that activity of naloxone and related drugs is not limited to peripheral systems and may interfere with the analgesic effects of opioid narcotics.

Inasmuch as post-surgical and post-partum ileus, for example, are common illnesses that add to the cost of health care and as yet have no specific treatments, there is a need for a specific and effective remedy. The majority of currently known opioid antagonist therapies is not peripherally selective and has the potential for undesirable side effects resulting from penetration into the CNS. Given the estimated 21 million inpatient surgeries and 26 million outpatient surgeries each year, and an estimate of 4.7 million patients experiencing post-surgical ileus, methods involving opioid antagonists that are not only specific for peripheral systems, but also specific for the gut, are desirable for treating post-surgical and post-partum ileus.

There is still an unfulfilled need for compounds that may be used in methods to agonize or antagonize opioid receptors, particularly for use in preventing or treating undesirable side effects associated with administering exogenous opioids. The present invention is directed to these, as well as other important ends.

### SUMMARY OF THE INVENTION

Accordingly, the present invention is directed, in part, to compounds of Formula Ia: 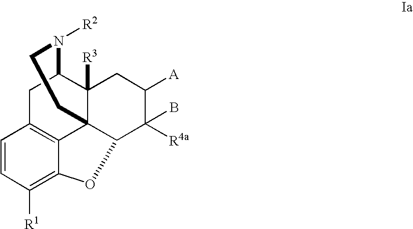
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³is —OR^{5f} or —N(R^{5g})—Y-Z;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A and B are each independently H or alkyl, or together represent a double bond between the carbon atoms to which they are attached;

R^{4a} is —[C(R^{5m})(R^{6m})]ₛ—R^{4b} or —OR⁵ⁿ, or R^{4a} and B taken together with the carbon atom to which they are attached may form: 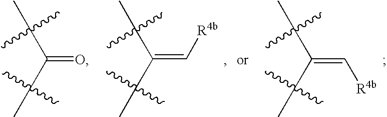
provided that when B is alkyl, then R^{4a} is —[C(R^{5m})(R^{6m})]ₛ—R^{4b};

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, and R^{5d} is independently H, alkyl, cycloalkyl, cycloalkylalkyl, or aralkyl;

R^{5c} is aralkyl;

R^{5e} is alkyl, cycloalkyl, cycloalkylalkyl, or aralkyl;

each R^{5f}, R^{5g}, R₅ₕ, R⁵ⁱ, R^{5j}, R^{5k}, R^{5m}, R⁵ⁿ, R^{5p}, R^{5q}, and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b} , R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently 0 or 1; and

each t is independently an integer from 1 to 12;

provided that:
(a) when R¹ is —OH or —O-alkyl, R² is H, cycloalkylalkyl, or alkenyl, R³ is —NR^{5g}—Y-Z, R^{5g} is H, alkyl, cycloalkylalkyl, or aralkyl, Y is —C(═O)— or a single bond, A is H, and either B is H and R^{4a} is —OR⁵ⁿ or B and R^{4a} taken together with the carbon atom to which they are attached form: 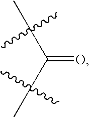
   then Z is heterocycloalkyl, heteroaryl, or —WR⁷;
(b) when R¹ and R³ are both —OH, R² is alkyl, cycloalkylalkyl, aryl, aralkyl, or alkenyl, A and B are each H, and R^{4a} is —NR^{5p}—C(═O)-Z, then R^{5p} is other than H, or Z is other than alkyl;
(c) when R¹ and R³ are both —OH, R² is alkyl, cycloalkylalkyl, aryl, aralkyl, or alkenyl, A and B are each H, and R^{4a} is —NR^{5p}-Z, then at least one of R^{5p} and Z is other than haloalkyl;
(d) when R¹ is —CON(R^{5d})(R^{6d}), then:
   at least one of R^{5d} and R^{6d} is cycloalkyl, or
   R^{6d} is aryl, or
   both R^{5d} and R^{6d} are other than H, or
   R^{5d} and R^{6d}, together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
(e) when R¹ and R³ are both —OH, R² is cyclopropylmethyl, and A and B together represent a double bond between the carbon atoms to which they are attached, then R^{4a} is other than phenyl, indolyl, or pyrrolyl;
(f) when R¹ and R³ are —OH, R² is cyclopropylmethyl, alkenyl, or alkyl, and R^{4a} and B taken together with the carbon atom to which they are attached form: 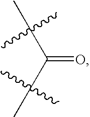
   then A is alkyl; and
(g) when R¹ and R³ are —OH, R² is cycloalkylalkyl, and A and B are both H, then R^{4a} is other than —NH₂ or —OH;
   or a pharmaceutically acceptable salt thereof.

The present invention is also directed, in part, to compounds of Formula Ib: 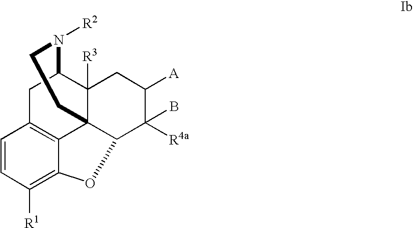
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —N(R^{5g})—Y-Z, wherein —N(R^{5g})—Y-Z is other than —NH₂;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)—or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A and B are each independently H or alkyl, or together represent a double bond between the carbon atoms to which they are attached;

R^{4a} is —[C(R^{5m})(R^{6m}))]ₛ—R^{4b} or —OR⁵ⁿ, or R^{4a} and B taken together with the carbon atom to which they are attached may form: 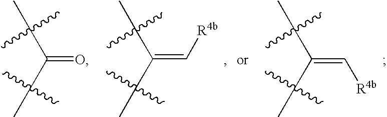
provided that when B is alkyl, then R^{4a} is —[C(R^{5m})(R^{6m})]ₛ—R^{4b};

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, R^{5c}, R^{5d}, and R^{5e} is independently H, alkyl, cycloalkyl, cycloalkylalkyl, or aralkyl;

each R^{5g}, R^{5h}, R⁵ⁱ, R^{5j}, R^{5k}, R^{5m}, R⁵ⁿ, R^{5p}, R^{5q}, and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently 0 or 1; and

each t is independently an integer from 1 to 12;

provided that:

when R¹ is —OH or —O-alkyl, R² is H, cycloalkylalkyl, or alkenyl, R^{5g} is H, alkyl, cycloalkylalkyl, or aralkyl, Y is —C(═O)— or a single bond, A is H, and either B is H and R^{4a} is —OR⁵ⁿ or B and R^{4a} taken together with the carbon atom to which they are attached form: 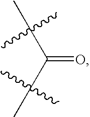
then Z is heterocycloalkyl, heteroaryl, or —WR⁷;
or a pharmaceutically acceptable salt thereof

The present invention is also directed, in part, to compounds of Formula Ic: 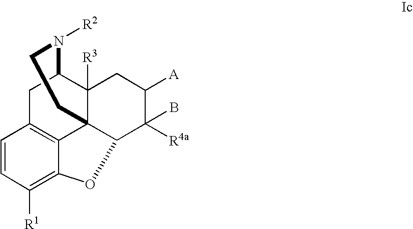
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —OR^{5f} or —N(R^{5g})—Y-Z;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A and B are each independently H or alkyl, or together represent a double bond between the carbon atoms to which they are attached;

R^{4a} is —[C(R^{5m})(R^{6m})]ₛ—R^{4b}, or

when A is H or alkyl, R^{4a} and B taken together with the carbon atom to which they are attached may form:

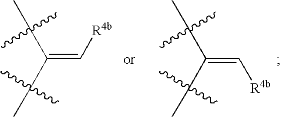

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, R^{5c}, R^{5d}, and R^{5e} is independently H, alkyl, cycloalkyl, cycloalkylalkyl, or aralkyl;

each R^{5f}, R^{5g}, R^{5h}, R⁵ⁱ, R^{5j}, R^{5k}, R^{5m}, R^{5p}, R^{5q}, and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d}, or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently 0 or 1; and

each t is independently an integer from 1 to 12;

provided that:
(a) when R^{4a} is —N(R^{5p})-Z, at least one of R^{5p} and Z is other than H or haloalkyl;
(b) when R¹ and R³ are both —OH, R² is alkyl, cycloalkylalkyl, aryl, aralkyl, or alkenyl, A and B are each H, and R^{4a} is —NR^{5p}—C(═O)-Z, then R^{5p} is other than H or Z is other than alkyl;
(c) when R¹ and R³ are both —OH, R² is cyclopropylmethyl, and A and B together represent a double bond between the carbon atoms to which they are attached, then R^{4a} is other than phenyl, indolyl, or pyrrolyl;
(d) when R¹ and R³ are both —OH, R² is alkyl, cycloalkylalkyl, aryl, aralkyl, or alkenyl, A and B are each H, and R^{4a} is —NR^{5p}-Z, then at least one of R^{5p} and Z is other than haloalkyl; and
(e) when R¹ and R³ are —OH, R² is cycloalkylalkyl, and A and B are both H, then R^{4a} is other than —NH₂;
   or a pharmaceutically acceptable salt thereof.

The present invention is also directed, in part, to compounds of Formula Id: 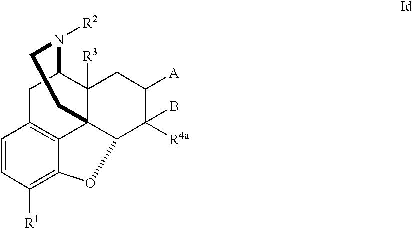
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —OR^{5f} or —N(R^{5g})—Y-Z;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A is H or alkyl and B is alkyl, or together A and B represent a double bond between the carbon atoms to which they are attached;

R^{4a} is —[C(R^{5m})(R^{6m})]ₛ—R^{4b}, or

when A is H or alkyl, R^{4a} and B taken together with the carbon atom to which they are attached may form:

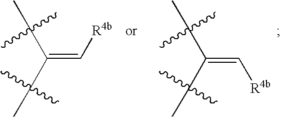

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, R^{5c}, R^{5d} and R^{5e} is independently H, alkyl, cycloalkyl, cycloalkylalkyl, or aralkyl;

each R^{5f}, R^{5g}, R^{5h}, R⁵ⁱ, R^{5j}, R^{5k}, R^{5m}, R⁵ⁿ, R^{5p}, R^{5q} and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m} and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently the integer 0 or 1; and

each t is independently an integer from 1 to 12;

provided that:
when R¹ and R³ are both —OH, R² is cyclopropylmethyl and A and B together represent a double bond between the carbon atoms to which they are attached, then R^{4a} is other than phenyl, indolyl, or pyrrolyl;
   or a pharmaceutically acceptable salt thereof

In other embodiments, the invention is directed to pharmaceutical compositions comprising a pharmaceutically acceptable carrier and a compound of Formula Ia, Ib, Ic, or Id.

In certain embodiments, the invention is directed to methods of preventing or treating a condition or disease associated with binding opioid receptors in a patient in need thereof, comprising the step of:

administering to said patient a composition comprising an effective amount of a compound of Formula Ia, Ib, Ic, or Id.

Another embodiment of the invention provides a method for treating or preventing a side effect associated with an opioid comprising the step of administering to a patient in need thereof, an effective amount of a compound of Formula Ia, Ib, Ic, or Id.

### DETAILED DESCRIPTION OF ILLUSTRATIVE EMBODIMENTS

As employed above and throughout the disclosure, the following terms, unless otherwise indicated, shall be understood to have the following meanings.

As used herein, “compounds of Formula I” collectively refers to compounds of Formula Ia, Ib, Ic, and Id, or any combination thereof.

As used herein, the term “alkyl” refers to an optionally substituted, saturated, straight or branched hydrocarbon having from about 1 to about 20 carbon atoms (and all combinations and subcombinations of ranges and specific numbers of carbon atoms therein), with from about 1 to about 8 carbon atoms, herein referred to as “lower alkyl,” being preferred. Alkyl groups include, but are not limited to, methyl, ethyl, n-propyl, isopropyl, n-butyl, isobutyl, sec-butyl, t-butyl, n-pentyl, isopentyl, neopentyl, n-hexyl, isohexyl, 3-methylpentyl, 2,2-dimethylbutyl, and 2,3-dimethylbutyl.

As used herein, the term “alkenyl” refers to an optionally substituted alkyl group having from about 2 to about 10 carbon atoms and one or more double bonds (and all combinations and subcombinations of ranges and specific numbers of carbon atoms therein), wherein alkyl is as previously defined. In some embodiments, it is preferred that the alkenyl groups have from about 2 to about 6 carbon atoms. Alkenyl groups may be optionally substituted.

As used herein, “alkylene” refers to a bivalent alkyl radical having the general formula —(CH₂)ₙ—, where n is 1 to 10. Non-limiting examples include methylene, trimethylene, pentamethylene, and hexamethylene.

As used herein, the term “alkynyl” refers to an optionally substituted alkyl group having from about 2 to about 10 carbon atoms and one or more triple bonds (and all combinations and subcombinations of ranges and specific numbers of carbon atoms therein), wherein alkyl is as previously defined.

As used herein, “aryl” and “aromatic” each refer to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

As used herein, the term “aralkyl” refers to an optionally substituted ring system comprising an alkyl radical bearing an aryl substituent and having from about 6 to about 50 carbon atoms (and all combinations and subcombinations of ranges and specific numbers of carbon atoms therein), with from about 6 to about 10 carbon atoms being preferred. Non-limiting examples include, for example, benzyl, diphenylmethyl, triphenylmethyl, phenylethyl, and diphenylethyl. In some preferred embodiments, the alkyl moieties of the aralkyl groups have from about 1 to about 4 carbon atoms. In other preferred embodiments, the alkyl moieties have from about 1 to about 3 carbon atoms.

As used herein, “aralkenyl” refers to an optionally substituted ring system comprising an alkenyl radical bearing an aryl substituent and have from about 7 to about 50 carbon atoms (and all combinations and subcombinations of ranges and specific numbers of carbon atoms therein) with from about 8 to about 15 carbon atoms being preferred, wherein aryl and alkenyl are as previously defined. Non-limiting examples include, for example, styryl, alpha-methylstyryl, beta-methylstyryl, 3-phenyl-1-propen-1-yl, 1-phenylprop-1-en-2-yl, alpha-naphthylethenyl, beta-naphthylethenyl, and diphenylethenyl.

As used herein, the term “heteroaryl” refers to an optionally substituted, mono-, di-, tri- or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Exemplary heteroaryl groups include, but are not limited to, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl. Heteroaryl may be optionally attached via a carbon or a heteroatom to the rest of the molecule.

As used herein, the term “cycloalkyl” refers to an optionally substituted alkyl group having one or more rings in their structures and having from about 3 to about 20 carbon atoms (and all combinations and subcombinations of ranges and specific numbers of carbon atoms therein), with from about 3 to about 8 carbon atoms being preferred. Multi-ring structures may be bridged or fused ring structures, wherein the additional groups fused or bridged to the cycloalkyl ring may include optionally substituted cycloalkyl, aryl, heterocycloalkyl, or heteroaryl rings. Exemplary cycloalkyl groups include, but are not limited to, cyclopropyl, cyclobutyl, cyclopentyl, cyclohexyl, cyclooctyl, adamantyl, 2-[4-isopropyl-1-methyl-7-oxa-bicyclo[2.2.1]heptanyl], and 2-[1,2,3,4-tetrahydro-naphthalenyl].

As used herein, the term “alkylcycloalkyl” refers to an optionally substituted ring system comprising a cycloalkyl group having one or more alkyl substituents, wherein cycloalkyl and alkyl are each as previously defined. Exemplary alkylcycloalkyl groups include 2-methylcyclohexyl, 3,3-dimethylcyclopentyl, trans-2,3-dimethylcyclooctyl, and 4-methyldecahydronaphthalenyl.

As used herein, the term “cycloalkylalkyl” refers to an optionally substituted ring system comprising an alkyl radical bearing a cycloalkyl substituent, and having from about 4 to about 50 carbon atoms (and all combinations and subcombinations of ranges and specific numbers of carbon atoms therein), with from about 6 to about 10 carbon atoms being preferred, wherein alkyl and cycloalkyl are as previously defined. In some preferred embodiments, the alkyl moieties of the cycloalkylalkyl groups have from about 1 to about 3 carbon atoms. Non-limiting examples include, for example, cyclopropylmethyl, cyclobutylethyl, cyclopentylpropyl, cyclohexylmethyl, 4-[4-methyldecahydronaphthalenyl]-pentyl, 3-[trans-2,3-dimethylcyclooctyl]-propyl, 2-[4-isopropyl-1-methyl-7-oxa-bicyclo[2.2.1]heptanyl]methyl, 2-[1,2,3,4-tetrahydro-naphthalenyl]ethyl, 2-cyclooctyl-1-methylethyl, and adamantylpropyl.

As used herein, “heteroaralkyl” refers to an optionally substituted ring system comprising an alkyl radical bearing a heteroaryl substituent, having from about 2 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

As used herein, “heterocycloalkyl” refers to an optionally substituted, mono-, di-, tri-, or other multicyclic aliphatic ring system that includes at least one, and preferably from 1 to about 4 sulfur, oxygen, or nitrogen heteroatom ring members. Heterocycloalkyl groups can have from about 3 to about 20 carbon atoms (and all combinations and subcombinations of ranges and specific numbers of carbon atoms therein), with from about 4 to about 10 carbons being preferred. In more preferred embodiments, the heterocycloalkyl groups have from about 4 to about 8 ring members, wherein 1 or 2 members are sulfur, oxygen, or nitrogen and the remaining members are carbon atoms. The heterocycloalkyl group may be unsaturated, and may also be fused to aromatic rings. Examples of heterocycloalkyl groups include, for example, tetrahydrofuranyl, tetrahydrothienyl, piperidinyl, pyrrolidinyl, isoxazolidinyl, isothiazolidinyl, pyrazolidinyl, oxazolidinyl, thiazolidinyl, piperazinyl, morpholinyl, piperadinyl, decahydroquinolyl, octahydrochromenyl, octahydro-cyclopenta[c]pyranyl, 1,2,3,4,-tetrahydroquinolyl, octahydro-[2]pyrindinyl, decahydro-cycloocta[c]furanyl, and imidazolidinyl. When a heterocycloalkyl ring is said to be interrupted by an additional heteroatom moiety, as used herein, this interruption of the ring refers to the replacement of a heterocycloalkyl ring carbon atom by the heteroatom moiety stated. For example, when a piperidine ring is interrupted by an oxygen atom, the resultant ring is morpholine; or when a pyrrolidine ring is interrupted by an S(═O)₂ moiety, the resultant ring is a thiazolidine 1,1-dioxide or an isothiazolidine 1,1-dioxide.

As used herein, the term “spiroalkyl” refers to an optionally substituted alkylene diradical, both ends of which are bonded to the same carbon atom of the parent group to form a spirocyclic group. The spirocyclic group, as herein defined, has 3 to 20 ring atoms, preferably with 3 to 10 ring atoms. Exemplary spiroalkyl groups taken together with its parent group include, but are not limited to, 1-(1-methyl-cyclopropyl)-propan-2-one, 2-(1-phenoxy-cyclopropyl)-ethylamine, and 1-methyl-spiro[4.7]dodecane.

As used herein, “halo” and “halogen” each refers to a fluoro, chloro, bromo, or iodo moiety attached to a compound of the invention. Preferably, “halo” and “halogen” refer to fluoro or chloro moieties.

Typically, substituted chemical moieties include one or more substituents that replace hydrogen. Exemplary substituents include, for example, halo (e.g., F, Cl, Br, I), alkyl, cycloalkyl, alkylcycloalkyl, cycloalkylalkyl, alkenyl, alkynyl, aralkyl, aryl, heteroaryl, heteroaralkyl, spiroalkyl, heterocycloalkyl, hydroxyl (—OH), nitro (—NO₂), cyano (—CN), amino (—NH₂), —N-substituted amino (—NHR″), —N,N-disubstituted amino (—N(R″)R″), oxo (═O), carboxy (—COOH), —O—C(═O)R″, —C(═O)R″, —OR″, —C(═O)OR″, -(alkylene)-C(═O)—OR″, —NHC(═O)R″, aminocarbonyl (—C(═O)NH₂), —N-substituted aminocarbonyl (—C(═O)NHR″), —N,N-disubstituted aminocarbonyl (—C(═O)N(R″)R″), thiol, thiolato (—SR″), sulfonic acid (—SO₃H), phosphonic acid (—PO₃H), —P(═O)(OR″)OR″, —S(═O)R″, —S(═O)₂R″, —S(═O)₂NH₂, —S(═O)₂ NHR″, —S(═O)₂NR″R″, —NHS(═O)₂R″, —NR″S(═O)₂R″, —CF₃, —CF₂CF₃, —NHC(═O)NHR″, —NHC(═O)NR″R″, —NR″C(═O)NHR″, —NR″C(═O)NR″R″, —NR″C(═O)R″ and the like. In relation to the aforementioned substituents, each moiety R″ can be, independently, any of H, alkyl, cycloalkyl, alkenyl, aryl, aralkyl, heteroaryl, or heterocycloalkyl, for example.

“Side effect” refers to a consequence other than the one(s) for which an agent or measure is used, as the adverse effects produced by a drug, especially on a tissue or organ system other then the one sought to be benefited by its administration. In the case, for example, of opioids, the term “side effect” may refer to such conditions as, for example, constipation, opioid-induced bowel dysfunction, nausea and/or vomiting.

As used herein, the term “effective amount” refers to an amount of a compound as described herein that may be therapeutically effective to inhibit, prevent, or treat the symptoms of particular disease, disorder, or side effect. Such diseases, disorders, and side effects include, but are not limited to, those pathological conditions associated with the administration of opioids (for example, in connection with the treatment and/or prevention of pain), wherein the treatment or prevention comprises, for example, inhibiting the activity thereof by contacting cells, tissues, or receptors with compounds of the present invention. Thus, for example, the term “effective amount,” when used in connection with opioids, for example, for the treatment of pain, refers to the treatment and/or prevention of the painful condition. The term “effective amount,” when used in connection with opioid antagonist compounds, refers to the treatment and/or prevention of side effects typically associated with opioids including, for example, such side effects as constipation, nausea, and/or vomiting, as well as other side effects, discussed in further detail below. The term “effective amount,” when used in connection with compounds active against gastrointestinal dysfunction, refers to the treatment and/or prevention of symptoms, diseases, disorders, and conditions typically associated with gastrointestinal dysfunction. The term “effective amount,” when used in connection with anti-ileus compounds, refers to the treatment and/or prevention of symptoms, diseases, disorders, and conditions typically associated with ileus. The term “effective amount,” when used in connection with compounds effective against obesity, refers to the treatment and/or prevention of the obese condition.

As used herein, the term “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term specifically encompasses veterinary uses.

As used herein, the expressions “in combination with,” “combination therapy,” and “combination products” refer, in certain embodiments, to the concurrent administration to a patient of opioids, an anesthetic agent (inhaled anesthetic, hypnotic, anxiolytic, neuromuscular blocker and opioid) and/or optional ingredients (antibiotics, antivirals, antifungals, anti-inflammatories, anesthetics and mixtures thereof) and the compounds of the invention, preferably compounds of formula Ia. When administered in combination, each component may be administered at the same time or sequentially in any order at different points in time. Thus, each component may be administered separately but sufficiently closely in time so as to provide the desired therapeutic effect.

As used herein, the term “dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

As used herein, the term “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein throughout may exist in alternate forms and such alternate forms are intended to be included within the scope of the compounds described and claimed in the present application. Accordingly, reference herein to compounds of formula I is intended to include reference to these alternate forms. For example, many amino-containing compounds can be used or prepared as an acid addition salt. Often such salts improve isolation and handling properties of the compound. For example, depending on the reagents, reaction conditions, and the like, compounds as described herein can be used or prepared, for example, as their hydrochloride or tosylate salts. Alternate forms of the compounds described herein also include, for example, isomorphic crystalline forms, all chiral and racemic forms, including stereoisomeric and partial stereoisomeric forms, N-oxides, hydrates, solvates, and acid salt hydrates.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein throughout that contain, for example, both amino and carboxy groups, also include reference to their corresponding zwitterions.

“Patient” refers to animals, including mammals, preferably humans.

“Prodrug” refers to compounds specifically designed to maximize the amount of active species that reaches the desired site of reaction, which are of themselves typically inactive or minimally active for the activity desired, but through biotransformation are converted into biologically active metabolites.

As used herein, the term “stereoisomers” refers to compounds that have identical chemical constitution, but differ as regards the arrangement of the atoms or groups in space.

As used herein, the term “partial stereoisomer” refers to stereoisomers having two or more chiral centers wherein at least one of the chiral centers has defined stereochemistry (i.e., R or S) and at least one has undefined stereochemistry (i.e., R or S). When the term “partial stereoisomers thereof” is used herein, it refers to any compound within the described genus whose configuration at chiral centers with defined stereochemistry centers is maintained and the configuration of each undefined chiral center is independently selected from R or S. For example, if a stereoisomer has three chiral centers and the stereochemical configuration of the first center is defined as having “S” stereochemistry, the term “or partial stereoisomer” thereof refers to stereoisomers having SRR, SRS, SSR, or SSS configurations at the three chiral centers, and mixtures thereof.

“N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

When any variable occurs more than one time in any constituent or in any formula, its definition in each occurrence is independent of its definition at every other occurrence. Combinations of substituents and/or variables are permissible only if such combinations result in stable compounds.

Each of the elements A, B, and R^{4a} in the morphinans of the invention as illustrated in Formula I may exist in independent trans and cis stereochemical isomeric configuration relative to element R³ within the cycloalkyl ring common to all four said elements. The term “trans” as used herein describes the spatial relationship between R³ and any of A, B, and R^{4a} and refers to the individual relationship between, for example, R³ and A, R³ and B, or R³ and R^{4a}, wherein said A, B, or R^{4a} substituent is positioned on the opposite side of the ring relative to the R³ substituent, whereas in the “cis” isomer, said A, B, or R^{4a} substituent and the R³ substituent are on the same side of the ring. The present invention contemplates the individual stereoisomers, as well as racemic or diastereomeric mixtures. In certain preferred compounds of the present invention, said A substituent is trans to R³. In certain other preferred compounds, said B substituent is cis to R³ and accordingly said R^{4a} substituent and the R³ substituent are in the trans orientation with respect to each other.

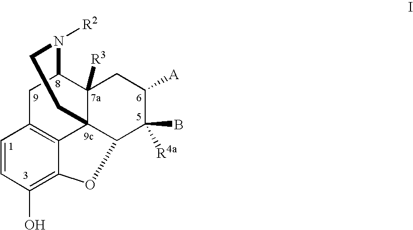

In addition to the “cis” and “trans” orientation of said A, B, or R^{4a} substituent and the R³ substituent, the absolute stereochemistry of the carbon atoms bearing said A, B, or R^{4a} substituent and the R³ substituent may also be defined using the commonly employed “R” and “S” definitions (Orchin et al., The Vocabulary of Organic Chemistry, 1980, John Wiley and Sons, Inc., page 126, which is incorporated herein by reference). The preferred compounds of the present invention are those of Formula I in which the configuration of the carbon atom bearing said R³ substituent is (S). The assignment of the stereochemical descriptor for the carbons bearing A and B will, by convention, depend upon the nature of the group selected for each of A, B, and R^{4a} and the relationship of A, B, and R^{4a} with R³, as noted above.

Furthermore, asymmetric carbon atoms may be introduced into the molecule depending on the structure of the moiety —W— when R⁵ⁱ and R⁶ⁱ are non-identical or in the moiety —[C(R^{5q})(R^{6q})]ₛ—C(═O)—N(R^{5r})—W—R⁷ when either R⁵ⁱ is non-identical to R⁶ⁱ in —W—, or R^{5q} is non-identical to R^{6q}, and the independent selection of any variables contained therein. For example, when R⁵ⁱ is hydrogen and R⁶ⁱ is other than H, the carbon atom to which R⁵ⁱ is attached is asymmetric.

Other asymmetric centers are contemplated in the present invention. Asymmetric centers are, by convention, present in the Formula I structure shown below at the ring carbon atoms identified as C-4a, C-5, C-6, C-7a, C-8 and C-9c. Further, independent selection of said 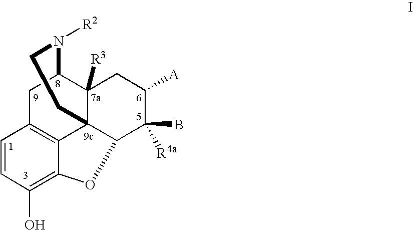
A, B, or R^{4a}, or independent sub-variables therein contained, may give rise to additional asymmetric centers. As such, these classes of compounds can exist as the individual “R” or “S” stereoisomers at each or any of these asymmetric centers, alone or in combination with any other asymmetric centers so formed in the compound to provide single enantiomers, any of the possible racemic mixtures of isomers or diastereomeric mixtures thereof, and all are contemplated as within the scope of the present invention. Preferably, a substantially pure stereoisomer of the compound of formula I is used, i.e., an isomer in which the configuration at the C-5 and C-6 asymmetric centers is independently “R” or “S”.

In certain preferred embodiments, the compounds, pharmaceutical compositions and methods of the present invention may involve a peripheral opioid antagonist compound. The term “peripheral” designates that the compound acts primarily on physiological systems and components external to the central nervous system. In preferred form, the peripheral opioid antagonist compounds employed in the methods of the present invention exhibit high levels of activity with respect to peripheral tissue, such as, gastrointestinal tissue, while exhibiting reduced, and preferably substantially no CNS activity. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5%, and most preferably 0%, of the pharmacological activity of the compounds employed in the present methods is exhibited in the CNS.

Furthermore, it is preferred in certain embodiments of the invention where the compound is administered to antagonize the peripheral side effects of an opioid that the compound does not substantially cross the blood-brain barrier and thereby decrease the beneficial activity of the opioid. The phrase “does not substantially cross,” as used herein, means that less than about 20% by weight of the compound employed in the present methods crosses the blood-brain barrier, preferably less than about 15% by weight, more preferably less than about 10% by weight, even more preferably less than about 5% by weight and most preferably 0% by weight of the compound crosses the blood-brain barrier. Selected compounds can be evaluated for CNS penetration by determining plasma and brain levels following i.v. administration.

Accordingly, in some embodiments, the present invention is directed to compounds of Formula Ia: 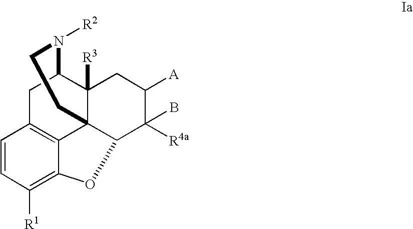
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —OR^{5f} or —N(R^{5g})—Y-Z;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A and B are each independently H or alkyl, or together represent a double bond between the carbon atoms to which they are attached;

R^{4a} is —[C(R^{5m})(R^{6m})]ₛ—R^{4b} or —OR⁵ⁿ, or R^{4a} and B taken together with the carbon atom to which they are attached may form: 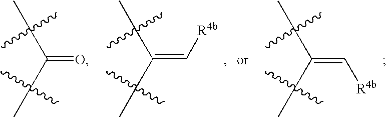
provided that when B is alkyl, then R^{4a} is —[C(R^{5m})(R^{6m})]ₛ—R^{4b};

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, and R^{5d} is independently H, alkyl, cycloalkyl, cycloalkylalkyl, or aralkyl;

R^{5c} is aralkyl;

R^{5e} is alkyl, cycloalkyl, cycloalkylalkyl, or aralkyl;

each R^{5f}, R^{5g}, R^{5h}, R⁵ⁱ, R^{5j}, R^{5k}, R^{5m}, R⁵ⁿ, R^{5p}, R^{5q}, and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently 0 or 1; and

each t is independently an integer from 1 to 12;

provided that:
(a) when R¹ is —OH or —O-alkyl, R² is H, cycloalkylalkyl, or alkenyl, R³ is —NR^{5g}—Y-Z, R^{5g} is H, alkyl, cycloalkylalkyl, or aralkyl, Y is —C(═O)— or a single bond, A is H, and either B is H and R^{4a} is —OR⁵ⁿ or B and R^{4a} taken together with the carbon atom to which they are attached form: 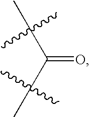
   then Z is heterocycloalkyl, heteroaryl, or —WR⁷;
(b) when R¹ and R³ are both —OH, R² is alkyl, cycloalkylalkyl, aryl, aralkyl, or alkenyl, A and B are each H, and R^{4a} is —NR^{5p}—C(═O)-Z, then R^{5p} is other than H, or Z is other than alkyl;
(c) when R¹ and R³ are both —OH, R² is alkyl, cycloalkylalkyl, aryl, aralkyl, or alkenyl, A and B are each H, and R^{4a} is —NR^{5p}-Z, then at least one of R^{5p} and Z is other than haloalkyl;
(d) when R¹ is —CON(R^{5d})(R^{6d}), then:
   at least one of R^{5d} and R^{6d} is cycloalkyl, or
   R^{6d} is aryl, or
   both R^{5d} and R^{6d} are other than H, or
   R^{5d} and R^{6d}, together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
(e) when R¹ and R³ are both —OH, R² is cyclopropylmethyl, and A and B together represent a double bond between the carbon atoms to which they are attached, then R^{4a} is other than phenyl, indolyl, or pyrrolyl;
(f) when R¹ and R³ are —OH, R² is cyclopropylmethyl, alkenyl, or alkyl, and R^{4a} and B taken together with the carbon atom to which they are attached form: 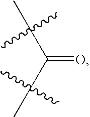
   then A is alkyl; and
(g) when R¹ and R³ are —OH, R² is cycloalkylalkyl, and A and B are both H, then R^{4a} is other than —NH₂ or —OH;
   or a pharmaceutically acceptable salt thereof.

In some embodiments, the present invention is directed to compounds of Formula Ib: 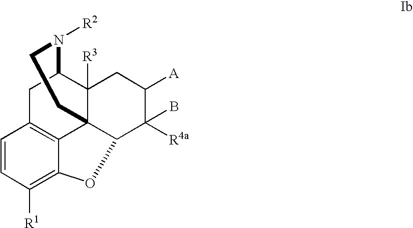
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —N(R^{5g})—Y-Z, wherein —N(R^{5g})—Y-Z is other than —NH₂;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A and B are each independently H or alkyl, or together represent a double bond between the carbon atoms to which they are attached;

R^{4a} is —[C(R^{5m})(R^{6m})]ₛ—R^{4b} or —OR⁵ⁿ, or R^{4a} and B taken together with the carbon atom to which they are attached may form: 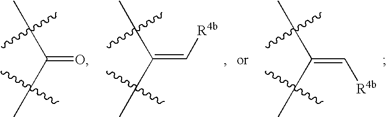
provided that when B is alkyl, then R^{4a} is —[C(R^{5m})(R^{6m})]ₛ—R^{4b};

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, R^{5c}, R^{5d}, and R^{5e} is independently H, alkyl, cycloalkyl, cycloalkylalkyl, or aralkyl;

each R^{5g}, R^{5h}, R⁵ⁱ, R^{5j}, R^{5k}, R^{5m} R⁵ⁿ, R^{5p}, R^{5q}, and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently 0 or 1; and

each t is independently an integer from 1 to 12;

provided that:
when R¹ is —OH or —O-alkyl, R² is H, cycloalkylalkyl, or alkenyl, R^{5g} is H, alkyl, cycloalkylalkyl, or aralkyl, Y is —C(═O)— or a single bond, A is H, and either B is H and R^{4a} is

—OR⁵ⁿ or B and R^{4a} taken together with the carbon atom to which they are attached form: 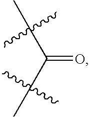
then Z is heterocycloalkyl, heteroaryl, or —WR⁷;
or a pharmaceutically acceptable salt thereof.

In some embodiments the invention is directed to compounds of Formula Ic: 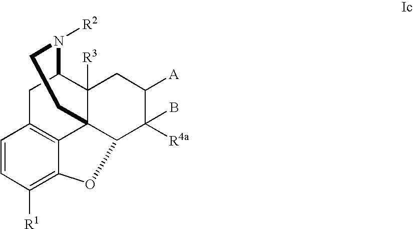
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —OR^{5f} or —N(R^{5g})—Y-Z;

each Y is independently a single bond, —[C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A and B are each independently H or alkyl, or together represent a double bond between the carbon atoms to which they are attached;

R^{4a} is —[C(R^{5m})(R^{6m})]ₛ—R^{4b}, or

when A is H or alkyl, R^{4a} and B taken together with the carbon atom to which they are attached may form:

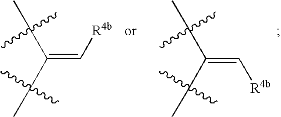

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, R^{5c}, R^{5d}, and R^{5e} is independently H, alkyl, cycloalkyl, cycloalkylalkyl, or aralkyl;

each R^{5f}, R^{5g}, R^{5h}, R⁵ⁱ, R^{5j}, R^{5k}, R^{5m}, R^{5p}, R^{5q} and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d}, or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently 0 or 1; and

each t is independently an integer from 1 to 12;

provided that:
(a) when R^{4a} is —N(R^{5p})-Z, at least one of R^{5p} and Z is other than H or haloalkyl;
(b) when R¹ and R³ are both —OH, R² is alkyl, cycloalkylalkyl, aryl, aralkyl, or alkenyl, A and B are each H, and R^{4a} is —NR^{5p}—C(═O)-Z, then R^{5p} is other than H or Z is other than alkyl;
(c) when R¹ and R³ are both —OH, R² is cyclopropylmethyl, and A and B together represent a double bond between the carbon atoms to which they are attached, then R^{4a} is other than phenyl, indolyl, or pyrrolyl;
(d) when R¹ and R³ are both —OH, R² is alkyl, cycloalkylalkyl, aryl, aralkyl, or alkenyl, A and B are each H, and R^{4a} is —NR^{5p}-Z, then at least one of R^{5p} and Z is other than haloalkyl; and
(e) when R¹ and R³ are —OH, R² is cycloalkylalkyl, and A and B are both H, then R^{4a} is other than —NH₂;
   or a pharmaceutically acceptable salt thereof.

In some embodiments the invention is directed to compounds of Formula Id: 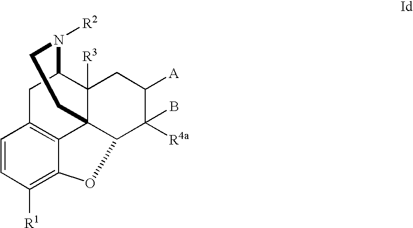
wherein:

R¹ is —OR^{5a}, —N(R^{5b})(R^{6b}), —COOR^{5c}, —CON(R^{5d})(R^{6d}), or —CH₂OR^{5e};

R² is H, alkyl, cycloalkyl, cycloalkylalkyl, aralkyl, aryl, or alkenyl;

R³ is —OR^{5f} or —N(R^{5g})—Y-Z;

each Y is independently a single bond, [—C(R^{5h})(R^{6h})]ₜ—, —C(═O)—, or —S(═O)₂—;

each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;

each W is independently —[C(R⁵ⁱ)(R⁶ⁱ)]ₜ—;

each R⁷ is independently —C(═O)—R⁸;

each R⁸ is independently —OR^{5j} or —N(R^{5k})(R^{6k});

A is H or alkyl and B is alkyl, or together A and B represent a double bond between the carbon atoms to which they are attached;

R^{4a} is —[C(R^{5m})(R^{6m})]ₛ—R^{4b}, or

when A is H or alkyl, R^{4a} and B taken together with the carbon atom to which they are attached may form:

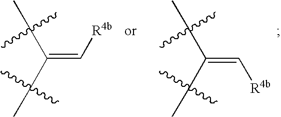

R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;

each R^{5a}, R^{5b}, R^{5c}, R^{5d} and R^{5e} is independently H, alkyl, cycloalkyl, cycloalkylalkyl, or aralkyl;

each R^{5f}, R^{5g}, R^{5h}, R⁵ⁱ, R^{5j}, R^{5k}, R^{5m}, R⁵ⁿ, R^{5p}, R^{5q} and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;

each R^{6b}, R^{6d}, R^{6h}, R⁶ⁱ, R^{6k}, R^{6m} and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;

each s is independently the integer 0 or 1; and

each t is independently an integer from 1 to 12;

provided that:
when R¹ and R³ are both —OH, R² is cyclopropylmethyl and A and B together represent a double bond between the carbon atoms to which they are attached, then R^{4a} is other than phenyl, indolyl, or pyrrolyl;
   or a pharmaceutically acceptable salt thereof.

In certain preferred embodiments, R¹ is —OR^{5a}, —CON(R^{5d})(R^{6d}), or —CH₂OR^{5e}. More preferably, R¹ is —OR^{5a}.

In some preferred embodiments, R² is alkyl, more preferably C₁₋₆ alkyl, or cycloalkylalkyl, more preferably cyclopropylmethyl.

In some preferred embodiments of compounds of Formula Ia, Ic, or Id, R³ is —N(R^{5g})—Y-Z.

In some preferred embodiments, each Y is independently a single bond, —[C(R^{5h})(R^{6h})]ₜ—, or —C(═O)—.

In some preferred embodiments, each Z is independently H, alkyl, heterocycloalkyl, aryl, or —WR⁷. In certain embodiments where Z is alkyl, it is preferably C₁₋₄ alkyl, more preferably butyl. In some embodiments where Z is heterocycloalkyl, it is preferably a six-membered ring heterocycle containing 1 or 2 ring heteroatoms, more preferably containing 1 or 2 nitrogen or oxygen atoms. Still more preferably the heterocycloalkyl is morpholinyl or piperidinyl. In some embodiments where Z is aryl, it is preferably phenyl, more preferably phenyl substituted with one or two chloro, methoxy, or alkoxycarbonyl, yet more preferably where the alkoxy of the alkoxycarbonyl is —O-C₁₋₆ alkyl. In other preferred embodiments, Z is: —WR⁷, preferably —W—C(═O)—R⁸, yet more preferably —W—C(═O)—OH, or alkyl, cycloalkyl, cycloalkylalkyl, heterocycloalkyl, aryl, or heteroaryl, said alkyl, cycloalkyl, cycloalkylalkyl, heterocycloalkyl, aryl, or heteroaryl groups being substituted with —CO₂H, or when R^{4b} is —N(R^{5p})—Y-Z, R^{5p} and Z together with the atoms through which they are connected form a 4- to 8-membered heterocycloalkyl ring, said heterocycloalkyl ring being substituted with —CO₂H.

In certain preferred embodiments, A is H or C₁₋₄ alkyl, preferably H or methyl, more preferably H. In certain preferred embodiments, B is H or C₁₋₄ alkyl, preferably H or methyl, more preferably H. In certain other preferred embodiments, A and B taken together represent a double bond between the carbon atoms to which they are attached.

In some preferred embodiments of compounds of Formula Ia, Ib, or Ic, A and B are each H.

In certain preferred embodiments, R^{4a} is —[C(R^{5m})(R^{6m})]ₛ—R^{4b}, or R^{4a} and B taken together form

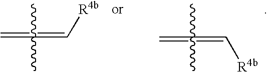

In some preferred embodiments of compounds of Formula Ia or Ib, R^{4a} and B taken together form;

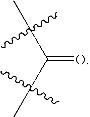

In some other preferred embodiments, R^{4b} is alkenyl, alkyl, aryl, heteroaryl, —N(R^{5p})—Y-Z, —C(═O)—R⁸, or —[C(R^{5q})(R^{6q})]ₛ—C(═O)—N(R^{5r})—W—R⁷. In certain of these embodiments, R^{4b} is alkenyl, preferably ethenyl. In certain other embodiments, R^{4b} is alkyl, preferably C₁₋₄ alkyl, more preferably substituted or unsubstituted methyl, yet more preferably CH₂OH. In some embodiments, R^{4b} is aryl, preferably phenyl, more preferably substituted phenyl, yet more preferably, phenyl substituted with one or two methyl, chloro, alkoxycarbonyl, hydroxy, alkoxy, phenoxy, trifluoromethyl, methanesulfonyl, or amino-, alkylamino- or dialkylamino-carbonyl. In other embodiments, R^{4b} is heteroaryl, preferably heteroaryl containing a ring nitrogen or oxygen atom, more preferably benzofuran or pyridyl.

In some more preferred embodiments, the compounds of the invention have the structure IIa or IIb: 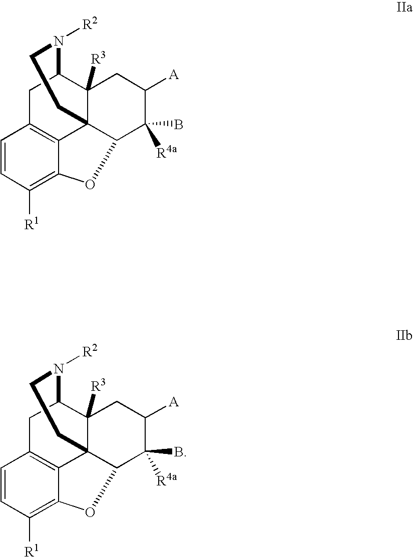
In other preferred embodiments, where the compounds according to Formula Ia or Ib have structure IIa or IIb, R^{4a} is —OR⁵ⁿ.

In certain embodiments, when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O) and S(═O)₂. In some other preferred embodiments, the heterocycloalkyl ring is substituted; more preferably substituted with carboxy or alkoxycarbonyl.

In some preferred embodiments of compounds of Formula Ib, Ic, or Id, each R^{5a}, R^{5b}, R^{5c}, R^{5d} and R^{5e} is independently H, alkyl, cycloalkyl, cycloalkylalkyl or aralkyl; more preferably, H or C₁₋₆ alkyl, yet more preferably H.

In some preferred embodiments, each R^{5b} and R^{5d} is independently H or alkyl, more preferably H or C₁₋₆ alkyl.

In some preferred embodiments, R^{5a} is H, alkyl, more preferably C₁₋₆ alkyl, cycloalkylalkyl, more preferably C₃₋₆ cycloalkylmethyl, or aralkyl, more preferably benzyl.

In some preferred embodiments of compounds of Formula Ia, Ic, or Id, R^{5f} is H.

In some preferred embodiments, R^{5j} is H or alkyl, more preferably H or C₁₋₆ alkyl.

In certain preferred embodiments, R^{5g}, R^{5h}, R⁵ⁱ, R^{5j}, R^{5k}, R^{5m}, R^{5p}, R^{5q} and R^{5r} is independently alkyl, cycloalkyl, cycloalkylalkyl, aryl, heteroaryl, or aralkyl.

In other preferred embodiments, each R^{5g}, R^{5h}, R⁵ⁱ, R^{5j}, R^{5k}, R^{5m}, R^{5p}, R^{5q}, and R^{5r} is independently H, alkyl, more preferably C₁₋₆ alkyl, or aryl, more preferably phenyl. In certain other preferred embodiments, R^{5g} is H. In still other preferred embodiments, R⁵ⁱ is H. In other preferred embodiments, R^{5j} is H. In certain preferred embodiments, R^{5k} is H. In some other preferred embodiments, R^{5r} is H. In still other preferred embodiments, R^{5p} is H.

In certain preferred embodiments of compounds of Formula Ia or Ib, each R^{5f}, R^{5g}, R^{5h}, R⁵ⁱ, R^{5j}, R^{5k}, R^{5m}, R⁵ⁿ, R^{5p}, R^{5q} and R^{5r} is independently H, alkyl, or aryl.

In some preferred embodiments of compounds of Formula Ia or Ib, R⁵ⁿ is H, alkyl, cycloalkyl, cycloalkylalkyl, aryl or aralkyl; more preferably H or C₁₋₆ alkyl; more preferably still H.

In some embodiments, each R^{6b}, R^{6d}, R^{6h}, R⁶ⁱ, R^{6k}, R^{6m} and R^{6q} is independently H, alkyl, aralkyl, or aryl. In certain embodiments, R^{6k} is alkyl or aralkyl; more preferably alkyl, yet more preferably C₁₋₆ alkyl. In other embodiments, R⁶ⁱ is H. In other embodiments, when R¹ is —N(R^{5b})(R^{6b}), then R^{5b} and R^{6b} together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂. In yet other embodiments, when R¹ is —CON(R^{5d})(R^{6d}), then R^{5d} and R^{6d} together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂. In yet other embodiments, R^{5d} and R^{6d} are each H. In still other embodiments, R^{5k} and R^{6k}, together with the nitrogen atom to which they are attached may form a 4- to 1 2-membered, more preferably 4- to 8-membered, heterocycloalkyl ring, said heterocycloalkyl ring optionally interrupted by one or more additional heteroatom moieties selected from nitrogen, oxygen, sulfur, S(═O), and S(═O)₂. In some other preferred embodiments, the heterocycloalkyl ring is substituted; more preferably substituted with carboxy or alkoxycarbonyl; yet more preferably the heterocycloalkyl ring is piperidinyl, still more preferably: 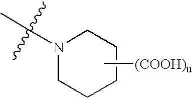
and u is 0 or 1. In some other preferred embodiments, R^{5q} and R^{6q} are each H.

In some preferred embodiments, each s is 0. In other preferred embodiments, each s is 1.

In some preferred embodiments of compounds of Formula Ia, Ib, Ic, or Id, each t the integer 1, 2, or 3, more preferably, 1 or 2, still more preferably 1. In other preferred embodiments, t is 3.

In some embodiments, the compounds of the invention are partial stereoisomers, prodrugs, pharmaceutically acceptable salts, hydrates, solvates, acid hydrates, or N-oxides thereof.

In certain embodiments, the compounds have a structure according to Formula III:

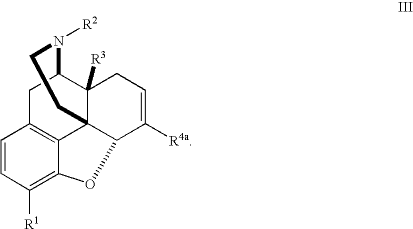

In certain embodiments, the compounds of the invention have a structure according to Formula IVa or IVb: 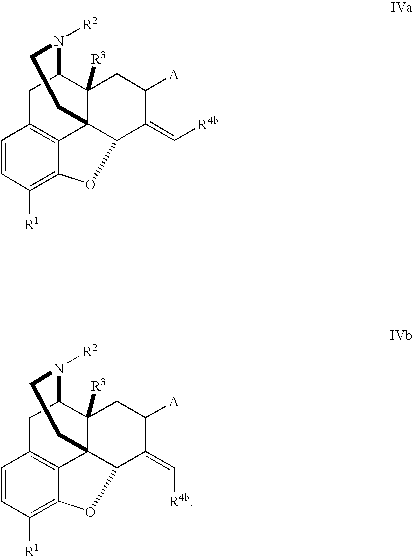
In some preferred embodiments, the compounds have structure IVa. In other preferred embodiments, the compounds have structure IVb.

In certain preferred embodiments of compounds of Formula Ia, the compound is selected from the group consisting of:

4,5α-Epoxy-6-carboxy-6,7-deshydro-3,14β-dihydroxy-17-(cyclopropylmethyl)-morphinan;

4,5α-Epoxy-6(α/β)-carboxy-3,14β-dihydroxy-17-(cyclopropylmethyl)morphinan;

4,5α-Epoxy-3,14β-dihydroxy-6,6Z(N-acryloylamino-acetic acid)-17-(cyclopropylmethyl)morphinan;

4,5α-Epoxy-3,14β-dihydroxy-6,6Z(N-acryloyl-piperidine-2(R/S)-carboxylic acid methyl ester)-17-(cyclopropylmethyl)morphinan;

4,5α-Epoxy-3,14β-dihydroxy-6,6Z(N-acryloyl-piperidine-3(R/S)-carboxylic acid)-17-(cyclopropylmethyl)morphinan;

4,5α-Epoxy-3,14β-dihydroxy-6,6E(carboxyethenyl)-17-(cyclopropylmethyl)morphinan;

4,5α-Epoxy-3,14β-dihydroxy-6,6E(N-acryloylamino-acetic acid)-17-(cyclopropylmethyl)morphinan;

4,5α-Epoxy-3,14β-dihydroxy-6,6E(N-acryloylamino-butyric acid)-17-(cyclopropylmethyl)morphinan;

4,5α-Epoxy-3,14β-dihydroxy-6,6E(N-acryloyl-piperidine-2(R/S)-carboxylic acid)-17-(cyclopropylmethyl)morphinan;

4,5α-Epoxy-3,14β-dihydroxy-6,6E(N-acryloyl-piperidine-3(R/S)-carboxylic acid)-17-(cyclopropylmethyl)morphinan;

4,5α-Epoxy-3,14β-dihydroxy-6,6E(N-acryloyl-piperidine-4-carboxylic acid)-17-(cyclopropylmethyl)morphinan;

4,5α-Epoxy-3-hydroxy-14β-acetylamino-6 β-carboxymethylamino-17-(cyclopropylmethyl)morphinan;

4,5α-Epoxy-3-hydroxy-14β-acetylamino-6 β-(4-carboxypiperidino)-17-(cyclopropylmethyl)morphinan;

4,5α-Epoxy-3-hydroxy-14β-acetylamino-6 β-(4-carboxybenzylamino)-17-(cyclopropylmethyl)morphinan;

4,5α-Epoxy-3-hydroxy-14β-[(4-carboxy)phenylcarbonylamino]-6-keto-17-(cyclopropylmethyl)morphinan;

4,5α-Epoxy-3-hydroxy-14β-[(4-carboxy)propionylamino]-6-keto-17-(cyclopropylmethyl)morphinan;

4,5α-Epoxy-3-hydroxy-14β-[(4-carboxy)propionylamino]-6-β-hydroxy-17-(cyclopropylmethyl)morphinan;

4,5α-Epoxy-3-hydroxy-14β-[(4-carboxy)propionylamino]-6-α-hydroxy-17-(cyclopropylmethyl)morphinan;

and stereoisomers, prodrugs, pharmaceutically acceptable salts, hydrates, solvates, acid hydrates, or N-oxides thereof.

In certain preferred embodiments of compounds of Formula Ia, the compound is selected from the group consisting of:

4,5α-Epoxy-3,14β-dihydroxy-6,6Z(N-acryloylamino-acetic acid)-17-(cyclopropylmethyl)morphinan;

4,5α-Epoxy-3,14β-dihydroxy-6,6Z(N-acryloyl-piperidine-2(R/S)-carboxylic acid methyl ester)-17-(cyclopropylmethyl)morphinan;

4,5α-Epoxy-3,14β-dihydroxy-6,6E(N-acryloylamino-acetic acid)-17-(cyclopropylmethyl)morphinan;

4,5α-Epoxy-3,14β-dihydroxy-6,6E(N-acryloylamino-butyric acid)-17-(cyclopropylmethyl)morphinan;

4,5α-Epoxy-3,14β-dihydroxy-6,6E(N-acryloyl-piperidine-2(R/S)-carboxylic acid)-17-(cyclopropylmethyl)morphinan;

4,5α-Epoxy-3,14β-dihydroxy-6,6E(N-acryloyl-piperidine-3(R/S)-carboxylic acid)-17-(cyclopropylmethyl)morphinan;

4,5α-Epoxy-3-hydroxy-14β-acetylamino-6 β-carboxymethylamino-17-(cyclopropylmethyl)morphinan;

4,5α-Epoxy-3-hydroxy-14β-[(4-carboxy)propionylamino]-6-keto-17-(cyclopropylmethyl)morphinan;

4,5α-Epoxy-3-hydroxy-14β-[(4-carboxy)propionylamino]-6-β-hydroxy-17-(cyclopropylmethyl)morphinan;

4,5α-Epoxy-3-hydroxy-14β-[(4-carboxy)propionylamino]-6-α-hydroxy-17-(cyclopropylmethyl)morphinan;

and stereoisomers, prodrugs, pharmaceutically acceptable salts, hydrates, solvates, acid hydrates, or N-oxides thereof.

In certain preferred embodiments of compounds of Formula Ia, the compound is selected from the group consisting of:

4,5α-Epoxy-3,14β-dihydroxy-6,6Z(N-acryloylamino-acetic acid)-17-(cyclopropylmethyl)morphinan;

4,5α-Epoxy-3,14β-dihydroxy-6,6E(N-acryloyl-piperidine-2(R/S)-carboxylic acid)-17-(cyclopropylmethyl)morphinan;

4,5α-Epoxy-3-hydroxy-14β-[(4-carboxy)propionylamino]-6-keto-17-(cyclopropylmethyl)morphinan;

4,5α-Epoxy-3-hydroxy-14β-[(4-carboxy)propionylamino]-6-β-hydroxy-17-(cyclopropylmethyl)morphinan;

4,5α-Epoxy-3-hydroxy-14β-[(4-carboxy)propionylamino]-6-α-hydroxy-17-(cyclopropylmethyl)morphinan;

and stereoisomers, prodrugs, pharmaceutically acceptable salts, hydrates, solvates, acid hydrates, or N-oxides thereof.

The compounds employed in the methods of the present invention may exist in prodrug form. As used herein, “prodrug” is intended to include any covalently bonded carriers which release the active parent drug, for example, as according to Formula Ia, Ib, Ic, or Id or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example Formula Ia, Ib, Ic, or Id, may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosure of which is hereby incorporated herein by reference, in its entirety.

Although the compounds of the present invention may be administered as the pure chemicals, it is preferable to present the active ingredient as a pharmaceutical composition. The invention thus further provides a pharmaceutical composition comprising one or more of the compounds of Formula Ia, Ib, Ic, or Id together with one or more pharmaceutically acceptable carriers therefore and, optionally, other therapeutic and/or prophylactic ingredients. The carrier(s) must be acceptable in the sense of being compatible with the other ingredients of the composition and not deleterious to the recipient thereof.

In accordance with certain embodiments of the present invention, the compositions of the invention may further comprise at least one opioid. A wide variety of opioids is available that may be suitable for use in the present methods and compositions. Generally speaking, it is only necessary that the opioid provide the desired effect (for example, pain alleviation), and be capable of being incorporated into the present combination products and methods (discussed in detail below). In preferred embodiments, the present methods and compositions may involve an opioid that is selected from alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil and/or tramadol. More preferably, the opioid is selected from morphine, codeine, oxycodone, hydrocodone, dihydrocodeine, propoxyphene, fentanyl, tramadol, and mixtures thereof.

The compositions of the invention may further comprise one or more other active ingredients that may be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosure of which is hereby incorporated herein by reference, in its entirety.

In addition, the compositions of the invention may further comprise one or more compounds that may be designed to enhance the analgesic potency of the opioid and/or to reduce analgesic tolerance development. Such compounds include, for example, dextromethorphan or other NMDA antagonists (Mao, M. J. et al., Pain, 1996, 67, 361), L-364,718 and other CCK antagonists (Dourish, C. T. et al., Eur. J. Pharmacol., 1988, 147, 469), NOS inhibitors (Bhargava, H. N. et al., Neuropeptides, 1996, 30, 219), PKC inhibitors (Bilsky, E. J. et al., J. Pharmacol. Exp. Ther., 1996, 277, 484), and dynorphin antagonists or antisera (Nichols, M. L. et al., Pain, 1997, 69, 317). The disclosures of each of the foregoing documents are hereby incorporated herein by reference, in their entireties.

Other opioids, optional conventional opioid components, and optional compounds for enhancing the analgesic potency of the opioid and/or for reducing analgesic tolerance development, that may be employed in the methods and compositions of the present invention, in addition to those exemplified above, would be readily apparent to one of ordinary skill in the art, once armed with the teachings of the present disclosure.

The compounds of the invention may be administered in an effective amount by any of the conventional techniques well-established in the medical field. The compounds employed in the methods of the present invention including, for example, the compounds of Formula Ia, Ib, Ic, or Id, may be administered by any means that results in the contact of the active agents with the agents' site or site(s)of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

In certain preferred embodiments, the compounds of the invention may be used in methods for preventing or treating post-operative or opioid-induced ileus.

Compounds of the present invention can be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The active compound may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations according to the present invention may be prepared so that an oral dosage unit form contains from about 0.1 to about 1000 mg of active compound.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying technique that yields a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

The combination products of this invention, such as pharmaceutical compositions comprising opioids in combination with the compounds of Formula Ia, Ib, Ic, or Id, may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the opioid compounds and the compounds of Formula Ia, Ib, Ic, or Id may be administered at the same time (that is, together), or in any order. When not administered at the same time, preferably the administration of an opioid and the compounds of Formula Ia, Ib, Ic, or Id occurs less than about one hour apart, more preferably less than about 30 minutes apart, even more preferably less than about 15 minutes apart, and still more preferably less than about 5 minutes apart. Preferably, administration of the combination products of the invention is oral, although other routes of administration, as described above, are contemplated to be within the scope of the present invention. Although it is preferable that the opioids and the compounds of Formula Ia, Ib, Ic, or Id are both administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Although the proper dosage of the combination products of this invention will be readily ascertainable by one skilled in the art, once armed with the present disclosure, by way of general guidance, where an opioid compounds is combined with the compounds of Formula Ia, Ib, Ic, or Id, for example, typically a daily dosage may range from about 0.01 to about 100 milligrams of the opioid (and all combinations and subcombinations of ranges therein) and about 0.001 to about 100 milligrams of the compounds of Formula Ia, Ib, Ic, or Id (and all combinations and subcombinations of ranges therein), per kilogram of patient body weight. Preferably, the a daily dosage may be about 0.1 to about 10 milligrams of the opioid and about 0.01 to about 10 milligrams of the compounds of Formula Ia, Ib, Ic, or Id per kilogram of patient body weight. Even more preferably, the daily dosage may be about 1.0 milligrams of the opioid and about 0.1 milligrams of the compounds of Formula Ia, Ib, Ic, or Id per kilogram of patient body weight. With regard to a typical dosage form of this type of combination product, such as a tablet, the opioid compounds (e.g., morphine) generally may be present in an amount of about 15 to about 200 milligrams, and the compounds of Formula Ia, Ib, Ic, or Id in an amount of about 0.1 to about 4 milligrams.

Particularly when provided as a single dosage form, the potential exists for a chemical interaction between the combined active ingredients (for example, an opioid and the compounds of Formula Ia, Ib, Ic, or Id). For this reason, the preferred dosage forms of the combination products of this invention are formulated such that although the active ingredients are combined in a single dosage form, the physical contact between the active ingredients is minimized (that is, reduced).

In order to minimize contact, one embodiment of this invention where the product is orally administered provides for a combination product wherein one active ingredient is enteric coated. By enteric coating one or more of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material that effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The dose may also be provided by controlled release of the compound, by techniques well known to those in the art.

Pharmaceutical kits useful in, for example, the treatment of pain, which comprise a therapeutically effective amount of an opioid along with a therapeutically effective amount of the morphinan compound of the invention, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL™ two-part container (available from Abbott Labs, Chicago, Ill.), as desired. The opioid compound and the compounds of Formula Ia, Ib, Ic, or Id may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

The compounds of the present invention may be used in methods to bind μ opioid receptors. Such binding may be accomplished by contacting the receptor with an effective amount of the compound of the invention. The opioid receptors may be located in the central nervous system or located peripherally to the central nervous system or in both locations. Preferably, the contacting step conducted in an aqueous medium, preferably at physiologically relevant ionic strength, pH, and the like.

In preferred embodiments of the methods of the invention, the compounds are opioid receptor agonists. In certain other preferred embodiments, the compounds prevent or treat a condition or disease caused by an opioid (either endogenous or exogenous). In certain embodiments of the method, particularly where the opioid are exogenous, the compounds of the invention preferably do not substantially cross the blood-brain barrier.

In other preferred embodiments of the methods of the invention, the compounds antagonize the activity of the opioid receptors. In other preferred embodiments, the compounds prevent or treat a condition or disease caused by an opioid (either endogenous or exogenous). In certain embodiments of the method, particularly where the opioid are exogenous, the compounds of the invention preferably do not substantially cross the blood-brain barrier.

The compounds of the present invention may be used in methods to antagonize l opioid receptors, particularly where undesirable symptoms or conditions are side effects of administering exogenous opioids. Furthermore, the compounds of the invention may be used as to treat patients having disease states that are ameliorated by binding opioid receptors or in any treatment wherein temporary suppression of the μ opioid receptor system is desired.

Such symptoms, conditions or diseases include the complete or partial antagonism of opioid-induced sedation, confusion, respiratory depression, euphoria, dysphoria, hallucinations, pruritus (itching), increased biliary tone, increased biliary colic, and urinary retention, ileus, emesis, and addiction liability; prevention or treatment of opioid and cocaine dependence; rapid opioid detoxification; treatment of alcoholism; treatment of alcoholic coma; detection of opioid use or abuse (pupil test); treatment of eating disorders; treatment of obesity; treatment of post-concussional syndrome; adjunctive therapy in septic, hypovolemic or endotoxin-induced shock; potentiation of opioid analgesia (especially at ultra-low doses); reversal or prevention of opioid tolerance and physical dependence (especially at ultra-low doses); prevention of sudden infant death syndrome; treatment of psychosis (especially wherein the symptoms are associated with schizophrenia, schizophreniform disorder, schizoaffective disorder, unipolar disorder, bipolar disorder, psychotic depression, Alzheimer's disease, Parkinson's disease, compulsive disorders, and other psychiatric or neurologic disorders with psychosis as symptoms); treatment of dyskinesia, treatment of autism; treatment of the endocrine system (including increased release of leutinizing hormone, treatment of infertility, increasing number of multiple births in animal husbandry, and male and female sexual behavior); treatment of the immune system and cancers associated with binding of the opioid receptors; treatment of anxiolysis; treatment of diuresis; treatment and regulation of blood pressure; treatment of tinnitus or impaired hearing; treatment of epilepsy; treatment of cachexia; treatment of general cognitive dysfunctions; and treatment of kleptomania.

The compounds of the present invention may also be used as cytostatic agents, as antimigraine agents, as immunomodulators, as immunosuppressives, as antiarthritic agents, as antiallergic agents, as virucides, to treat diarrhea, antipsychotics, as antischizophrenics, as antidepressants, as uropathic agents, as antitussives, as antiaddictive agents, as anti-smoking agents, to treat alcoholism, as hypotensive agents, to treat and/or prevent paralysis resulting from traumatic ischemia, general neuroprotection against ischemic trauma, as adjuncts to nerve growth factor treatment of hyperalgesia and nerve grafts, as anti-diuretics, as stimulants, as anti-convulsants, or to treat obesity. Additionally, the present compounds may be used in the treatment of Parkinson's disease as an adjunct to L-dopa for treatment dyskinesia associated with the L-dopa treatment.

In certain preferred embodiments, the compounds of the invention may be used in methods for preventing or treating gastrointestinal dysfunction, including, but not limited to, irritable bowel syndrome, opioid-bowel dysfunction, colitis, post-operative and opioid-induced emesis (nausea and vomiting), decreased gastric motility and emptying, inhibition of small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, and delayed absorption of orally administered medications or nutritive substances.

In yet other embodiments, the invention is directed to methods of preventing or treating a condition or disease associated with binding opioid receptors in a patient in need thereof, comprising the step of:

administering to said patient a composition comprising an effective amount of a compound of Formula Ia, Ib, Ic, or Id.

Still another embodiment of the invention provides a method for treating or preventing ileus comprising the step of administering to a patient in need of such treatment, an effective amount of a compound of Formula Ia, Ib, Ic, or Id. In some preferred embodiments, the ileus is post-operative ileus.

While not intending to be bound by any theory or theories of operation, it is contemplated that opioid side effects, such as constipation, opioid-induced bowel dysfunction, vomiting and nausea, may result from undesirable interaction of the opioid with peripheral opioid receptors, such as peripheral μ receptors. Administration of the compounds of Formula Ia, Ib, Ic, or Id according to one aspect of the present invention may block interaction of the opioid compounds with the peripheral receptors, thereby preventing and/or inhibiting the side effects, while preferably not interfering with the therapeutic effect of the opioid in the central nervous system (CNS).

Another embodiment of the invention provides a method for treating or preventing a side effect associated with an opioid comprising the step of administering to a patient, a composition comprising an effective amount of a compound of Formula Ia, Ib, Ic, or Id. In certain preferred embodiments, the opioid is endogenous. In other preferred embodiments, the opioid is exogenous. In still other preferred embodiments, the composition further comprises an effective amount of at least one opioid.

In still other preferred embodiments, the invention provides a method for preventing or treating pain, comprising the step of:

administering to a patient in need thereof an effective amount of a compound of Formula Ia, Ib, Ic, or Id.

In other preferred embodiments, the invention provides a method for preventing or treating pain, comprising the step of:

administering to a patient in need thereof an effective amount of a compound of Formula Ia, Ib, Ic, or Id and an effective amount of an opioid.

The compounds of the invention may be administered before, during or after administering at least one opioid. The methods of the invention are particularly effective for opioids selected from alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol, or mixtures thereof.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As discussed in detail above, compounds employed in the present methods may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups present may contain protecting groups during the course of synthesis. Protecting groups are known per se as chemical functional groups that can be selectively appended to and removed from functionalities, such as hydroxyl groups and carboxy groups. These groups are present in a chemical compound to render such functionality inert to chemical reaction conditions to which the compound is exposed. Any of a variety of protecting groups may be employed with the present invention. Preferred protecting groups include the benzyloxycarbonyl group and the tert-butyloxycarbonyl group. Other preferred protecting groups that may be employed in accordance with the present invention may be described in Greene, T. W. and Wuts, P. G. M., Protective Groups in Organic Synthesis 2d. Ed., Wiley & Sons, 1991.

The morphinan compounds according to the present invention may be synthesized employing methods taught, for example, in U.S. Pat. No. 5,250,542, U.S. Pat. No. 5,434,171, U.S. Pat. No. 5,159,081, and U.S. Pat. No. 5,270,328, the disclosures of which are hereby incorporated herein by reference in their entireties. The optically active and commercially available Naltrexone (1, Scheme 1) was employed as starting material in the synthesis of the present compounds may be prepared by the general procedure taught in U.S. Pat. No. 3,332,950, the disclosure of which is hereby incorporated herein by reference in its entireties.

Employing the methodology herein described or cited, morphinan compounds of Formula Ia, Ib, Ic, or Id can be readily prepared. The invention is further described in the following examples. The actual examples, herein provided, are for illustrative purposes only, and are not to be construed as limiting the appended claims. They provide a series of 4,5α-epoxy-3,14β,17,6-(α/β) tetrasubstituted morphinan derivatives of Formulae I, Ia, IIb, III, IVa and IVb, prepared according to Schemes 1-12, shown below (Examples 1-87).

Examples 1-12 were prepared in four steps sequence from naltrexone (1). Naltrexone (1) was converted to the silyl ether 2 using tert-butyldimethylsilyl chloride. Conversion of the ketone 2 to the enol triflate derivative 3 was achieved using N-phenyl bis(trifluoromethanesulphonimide) 4 as triflating reagent. Suzuki type coupling of the enol triflate derivative 3 with various boronic acid derivatives 5a-5l in ethylene glycol dimethyl ether (DME) in the presence of tetrakis triphenylphosphine palladium (0), lithium chloride and an aqueous solution of sodium carbonate afforded compounds 6a-6l which were converted to the final products 7a-7l (Examples 1-12) using a solution of tert-butylammonium fluoride (TBAF) in tetrahydrofuran.

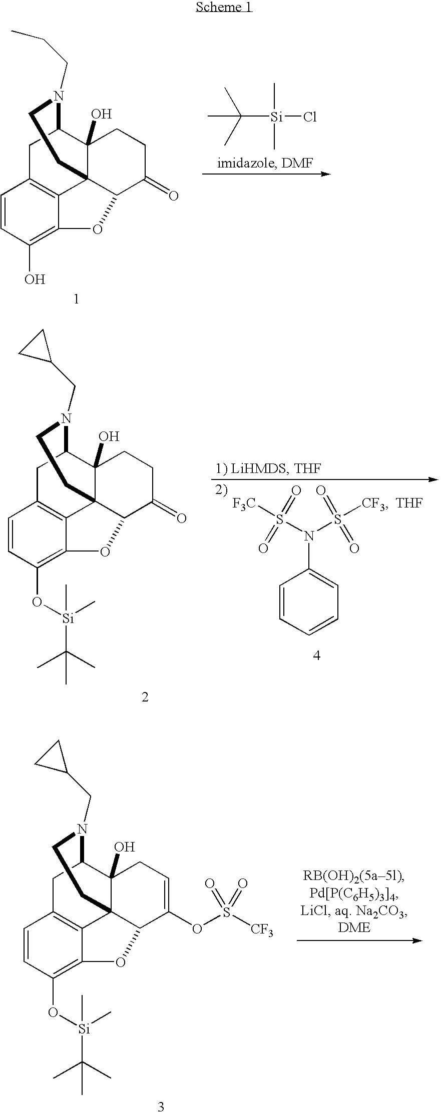

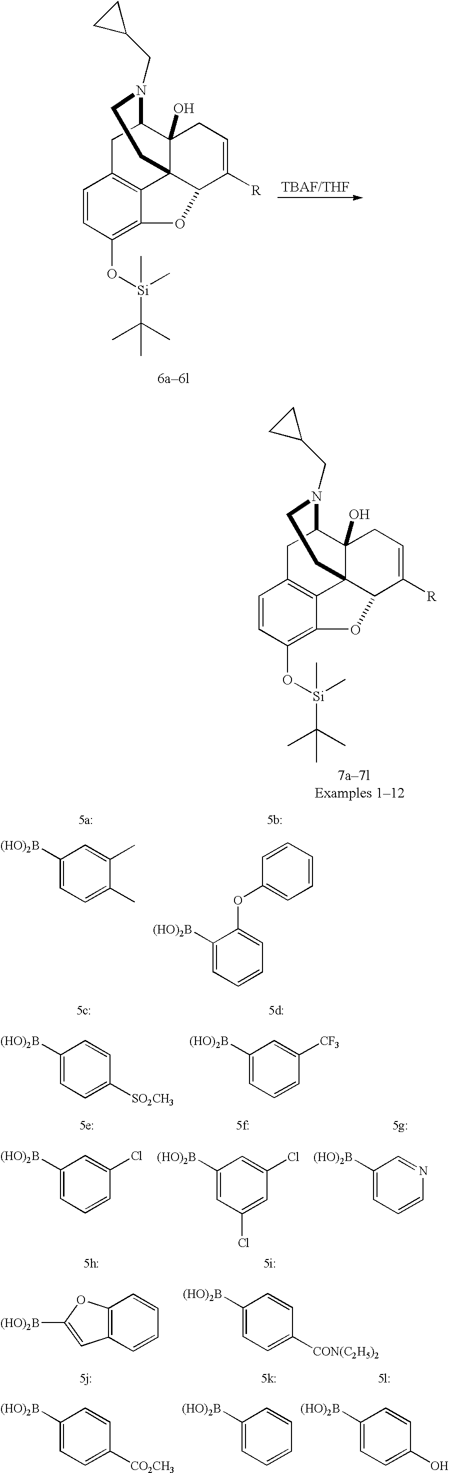

Examples 13-29 were each prepared in a three step sequence from the corresponding enol triflate derivative 3 (Scheme 2). Palladium catalyzed carbonylation of 3 provided the methyl ester 8, which was hydrolyzed under basic conditions to give the carboxylic acid 9 (Example 13). Coupling of 9 with various primary or secondary amines 10a-10m in the presence of triethylamine, using coupling reagent benzotriazol-1-yloxy-tris(dimethylamino)-phosphonium hexafluorophosphate (BOP) afforded the carboxamides 11a-11m (Examples 14-26). Note: The amines 10a-10i, 10l and 10m are commercially available; the amine 10j was prepared as reported in the patent literature [Gottschlich, R.; et al.; 1995, EP670318]; the (+)-4(R)-(3-hydroxyphenyl)-3(R),4-dimethyl-1-piperidine 10k was prepared as described in the following publication [Mitch, C. H., et al. J. Org. Chem. 1991, 56, 1660].

Treatment of the silyl ether 8 with a solution tert-butylammonium fluoride (TBAF) in tetrahydrofuran afforded the phenolic derivative 12 (Example 27), which was converted to 13 (Example 28) (diastereomeric mixture) by hydrogenation using palladium on carbon as catalyst. The carboxylic acid 14 (Example 29) was obtained similarly by hydrogenation of 9 using palladium on carbon as catalyst.

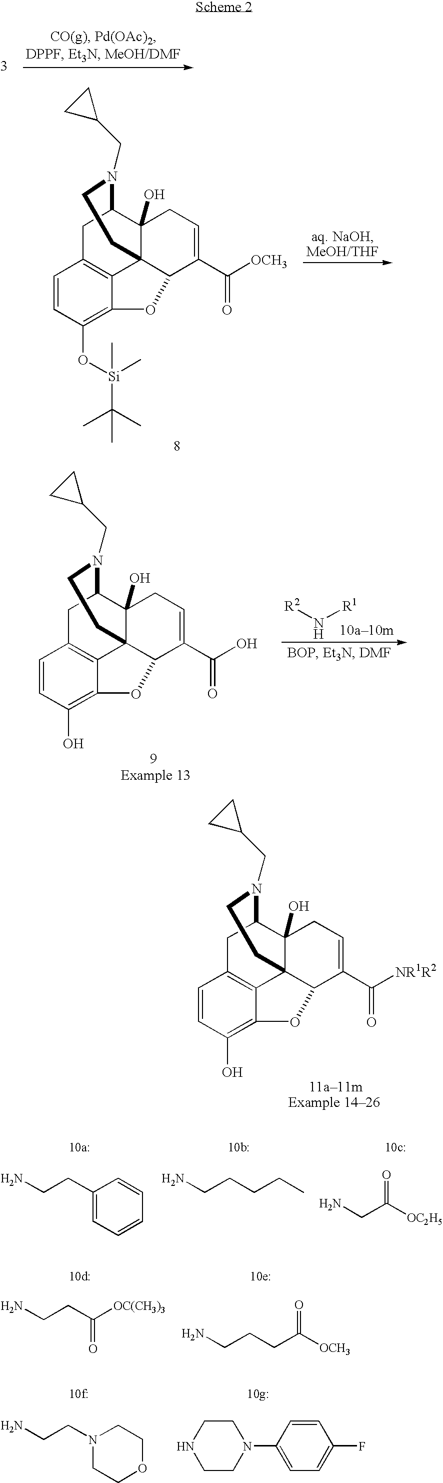

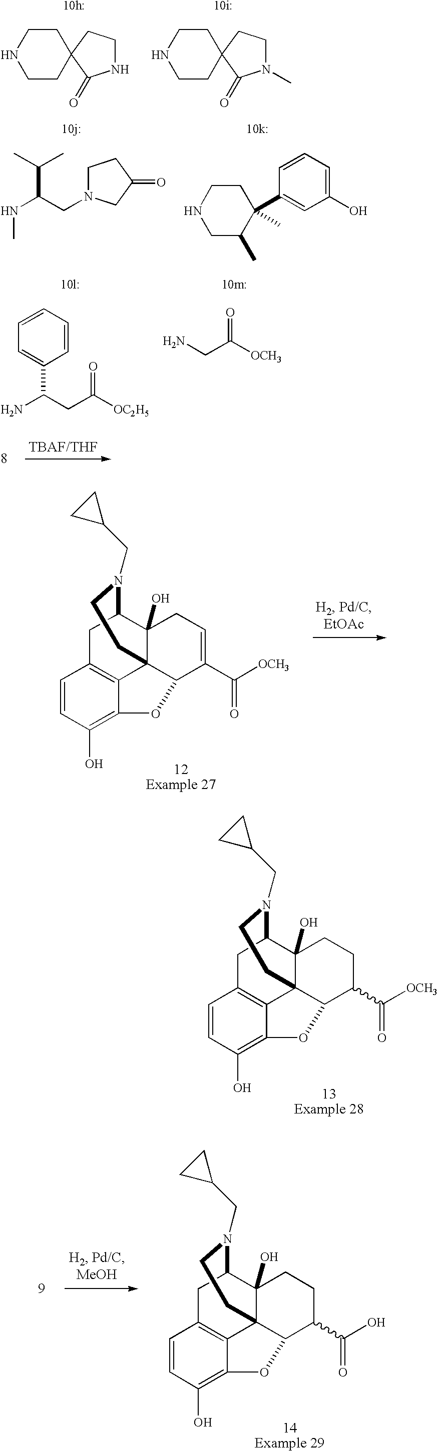

Examples 30-37 were each prepared in a three or four step sequence from naltrexone 1 (Scheme 3). The Wittig type condensation of naltrexone 1 with methyl diethylphosphonoacetate 15 in ethylene glycol dimethyl ether (DME) in the presence of sodium hydride gave a mixture of esters 16 (Example 30) and 17 (Example 31) in a ratio 16/17=1:3. The esters 16 and 17 were separated by silica gel flash column chromatography. Hydrolysis of the ester 16 under basic conditions provided the carboxylic acid derivative 18. Coupling of 18 with glycine methyl ester (10m), ethyl 2-piperidinecarboxylate (19a) or ethyl 3-piperidinecarboxylate (19b) in the presence of triethylamine, using benzotriazol-1-yloxy-tris(dimethylamino)-phosphonium hexafluorophosphate (BOP) as coupling reagent afforded the carboxamides 20a (Example 32), 20b (Example 34) and 20c (Example 36), respectively. Hydrolysis of the esters 20a-20c under basic conditions afforded the corresponding carboxylic acids 21a (Example 33), 21b (Example 35) and 21c (Example 37), respectively.

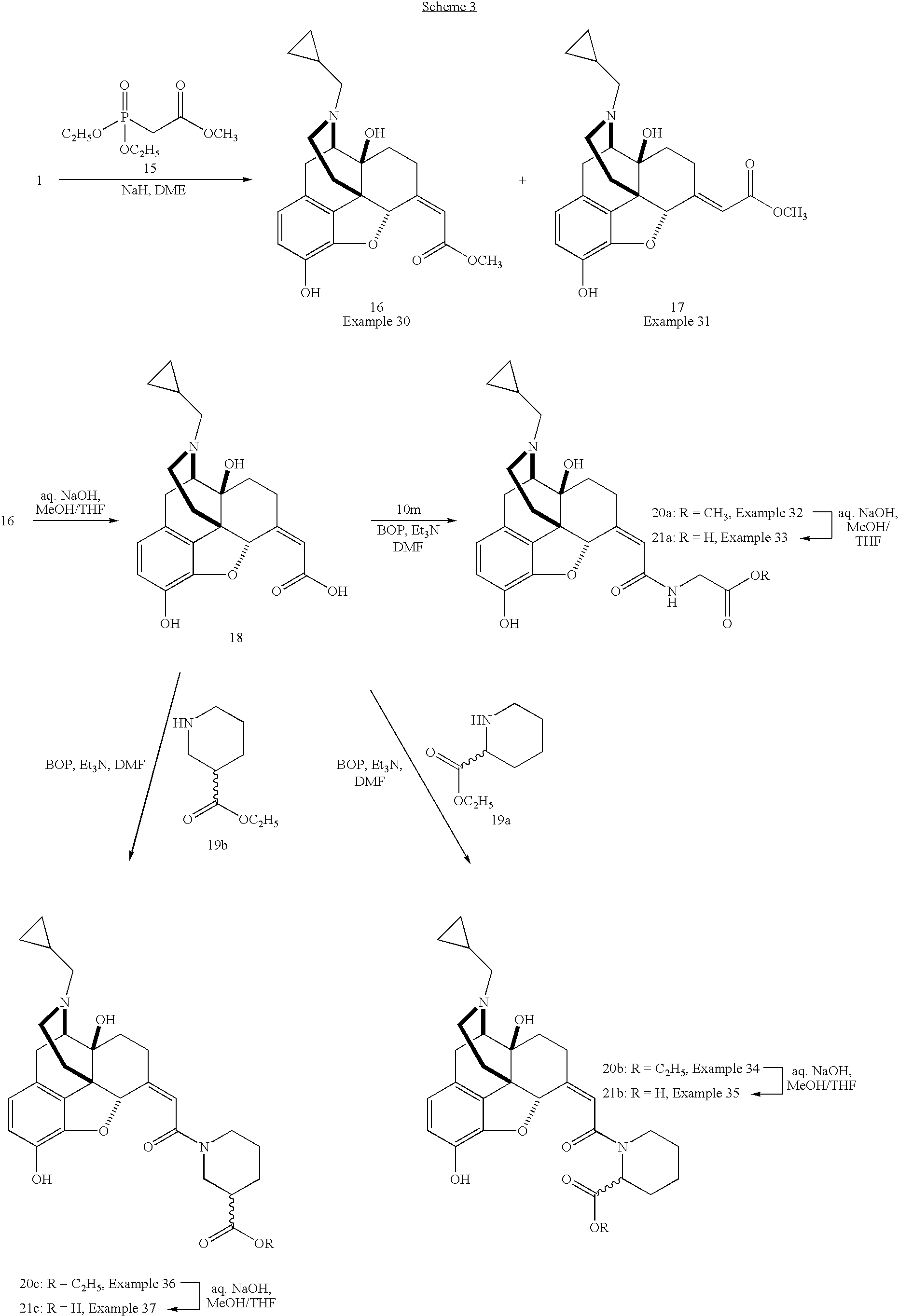

Examples 38-48 were prepared according to Scheme 4. Hydrolysis of the ester 17 under basic conditions provided the carboxylic acid derivative 22 (Example 38). Coupling of 22 with glycine methyl ester (10m), methyl 4-aminobutyrate (23a), ethyl 2-piperidinecarboxylate (19a), ethyl 3-piperidinecarboxylate (19b) or ethyl 4-piperidinecarboxylate (23b) in the presence 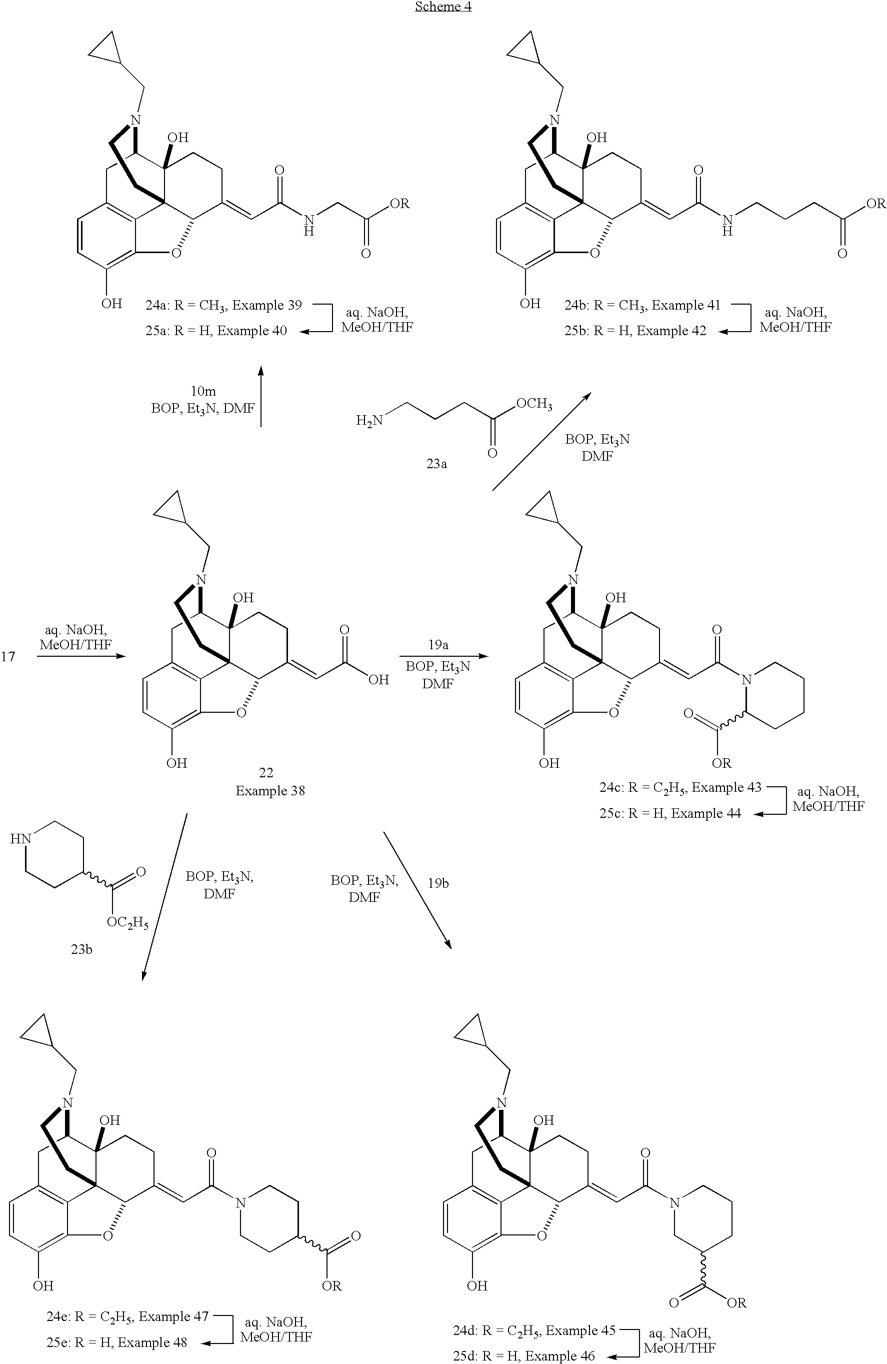
of triethylamine, using coupling reagent benzotriazol-1yloxy-tris(dimethylamino)-phosphonium hexafluorophosphate (BOP) afforded the carboxamides 24a (Example 39), 24b (Example 41), 24c (Example 43), 24d (Example 45) and 24e (Example 47), respectively. Hydrolysis of the esters 24a-24e under basic conditions afforded the corresponding carboxylic acids 25a (Example 40), 25b (Example 42), 25c (Example 44), 25d (Example 46) and 25e (Example 48), respectively.

Coupling of 22 with 3-piperidinemethanol (a), 4-piperidinemethanol (26b), 3-piperidinecarboxamide (26c), 4-piperidinecarboxamide (26d) or 2-methoxyethylamine (26e) in the presence of triethylamine, using benzotriazol-1-yloxy-tris(dimethylamino)-phosphonium hexafluorophosphate (BOP) as coupling reagent afforded the carboxamides 27a-27e respectively (Examples 49-53) (Scheme 5). The synthesis of Example 54 (diastereomeric mixture) is described in Scheme 6. The hydrolysis of the mixture of esters 16 and 17 under basic conditions provided the carboxylic acid derivatives 18 and 22. Coupling of 18/22 with glycine methyl ester 10m in the presence of the BOP reagent provided a mixture of the amides 20a/24a, which was converted to 28 (Example 54) by catalytic hydrogenation.

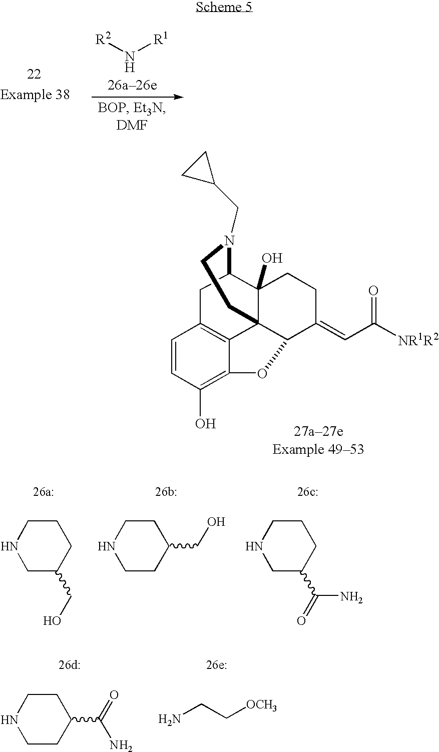

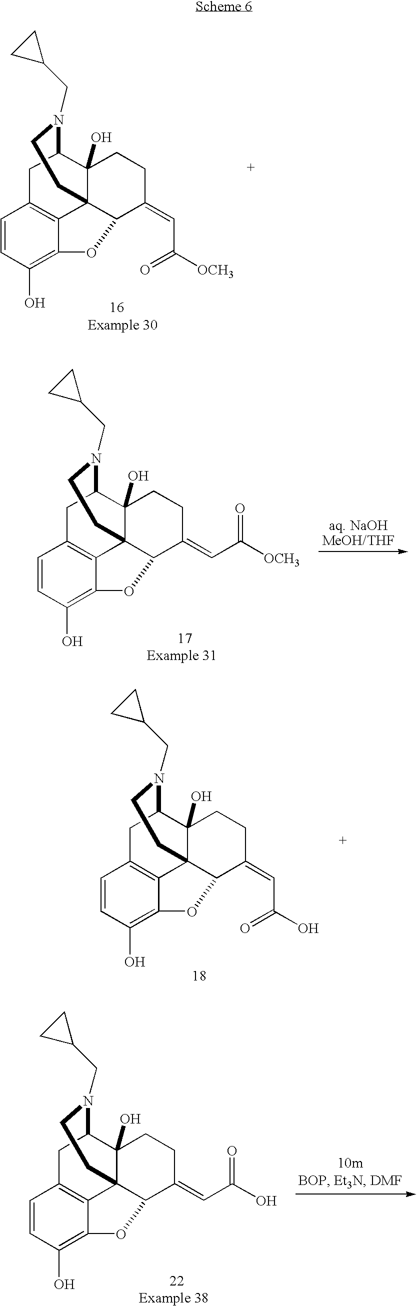

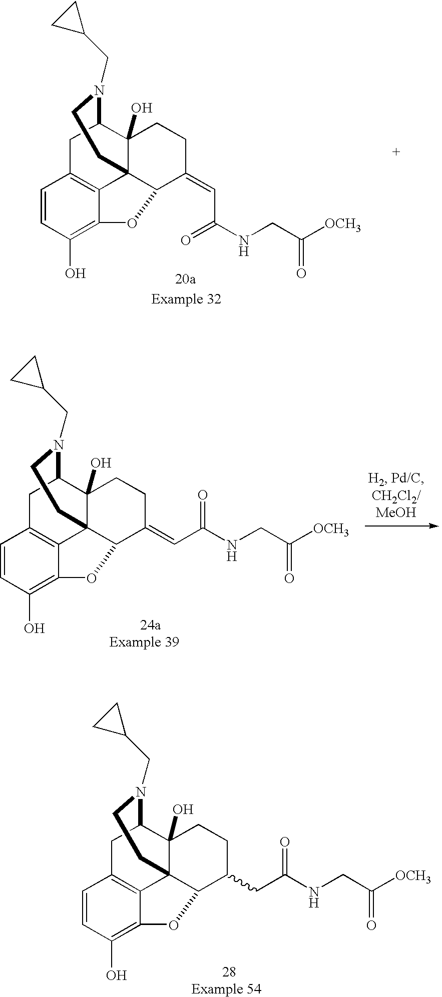

The synthesis of the primary alcohol derivatives (Example 55 and Example 56) is described in Scheme 7. The Wittig type condensation of the ketone 2 with methyl diethylphosphonoacetate 15 in ethylene glycol dimethyl ether in the presence of sodium hydride gave the mixture of esters 29 and 30. The esters 29 and 30 were separated by silica gel flash column chromatography. The ester functionality of 29 was subsequently reduced using diisobutylaluminium hydride. The resulting alcohol 31 was converted to the phenolic derivative 32 (Example 55) using a solution tert-butylammonium fluoride (TBAF) in tetrahydrofuran. Similarly, the ester functionality of 30 was reduced using diisobutylaluminium hydride. The resulting alcohol 33 was converted to the phenolic derivative 34 (Example 56) using a solution tert-butylammonium fluoride (TBAF) in tetrahydrofuran.

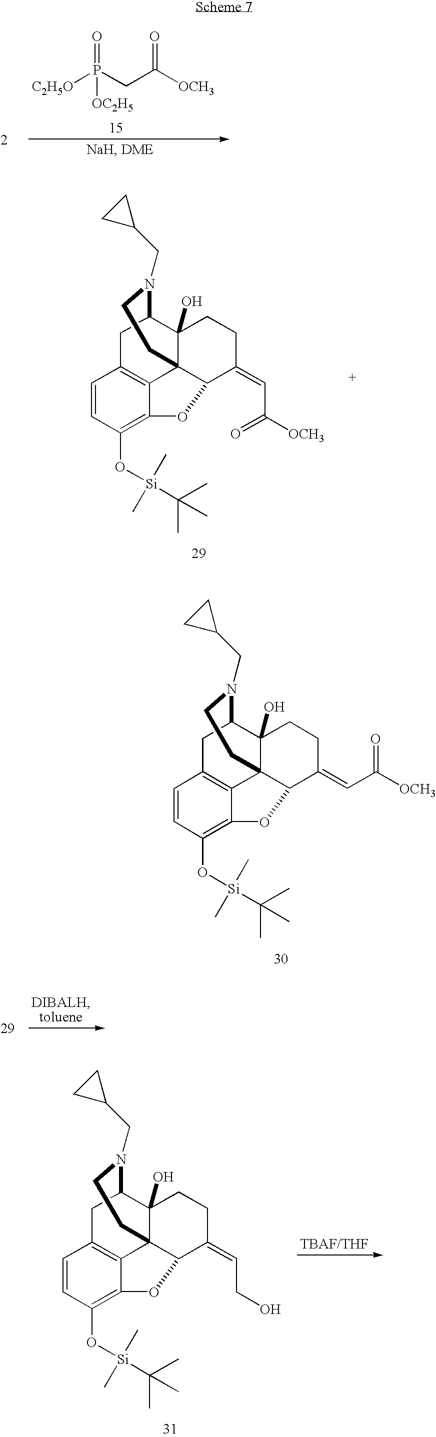

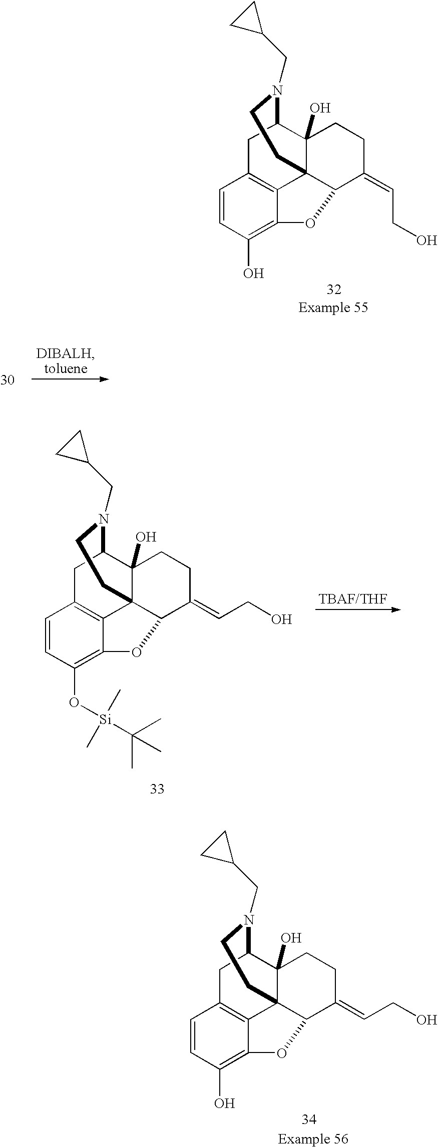

The synthesis of Example 57 is described in Scheme 8. Stille type cross coupling reaction of the enol triflate derivative 3 with vinyltributyltin 35 catalyzed by tetrakis triphenylphosphine palladium (0) afforded the silyl ether 36 which was converted to 37 (Example 57) using a solution tert-butylammonium fluoride (TBAF) in tetrahydrofuran.

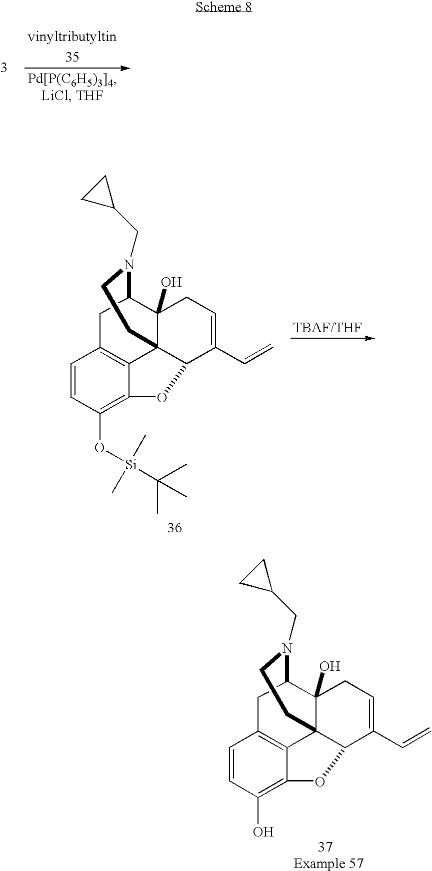

The synthesis of Examples 58-87 is described in Schemes 9-12. The ketone 38 (Kobylecki, R. J., et al., U.S. Pat. No. 4,241,066 (1980) reacted with various amines 39a-39o under reductive amination conditions using sodium cyanoborohydride as reducing agent to provide the 6β-(40a-o; Examples 58-72) or 6α-(41a-b; 41e-j; Examples 73-80) substituted 4,5α-epoxy-3-hydroxy-14β-acetylamino-17-(cyclopropylmethyl)morphinan (Scheme 9). Hydrolysis of the esters 40i, 40o and 40d provided the corresponding carboxylic acids 42a (Example 81), 42b (Example 82) and 42c (Example 83), respectively (Scheme 10).

Coupling of 43 with monomethylterephthalate 44, mediated by HATU, afforded the mixture of the amido-ester 45 and the amide 46. Hydrolysis of 45/46 in basic conditions provided the carboxylic acid 47 (Example 84) (Scheme 11). Coupling of 43 with monomethylglutarate 48, mediated by HATU, afforded the mixture of the amido-ester 49 and the amide 50. Hydrolysis of 49/50 in basic conditions provided the carboxylic acid 51 (Example 85) (Scheme 12). Reduction of the ketone functionality of 51 using sodium borohydride provided the secondary alcohols 52 (Example 86) and 53 (Example 87), separated by HPLC.

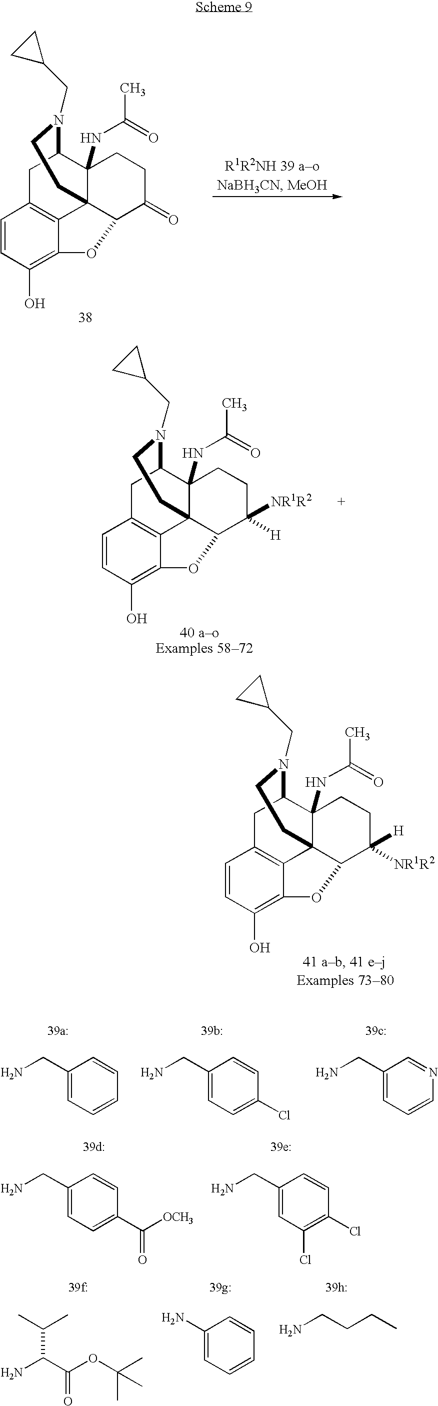

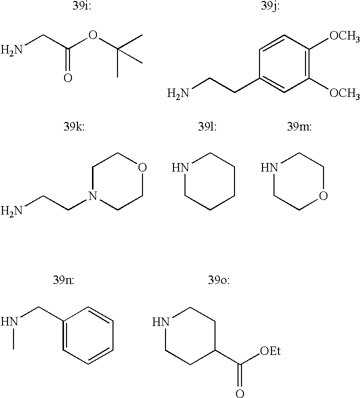

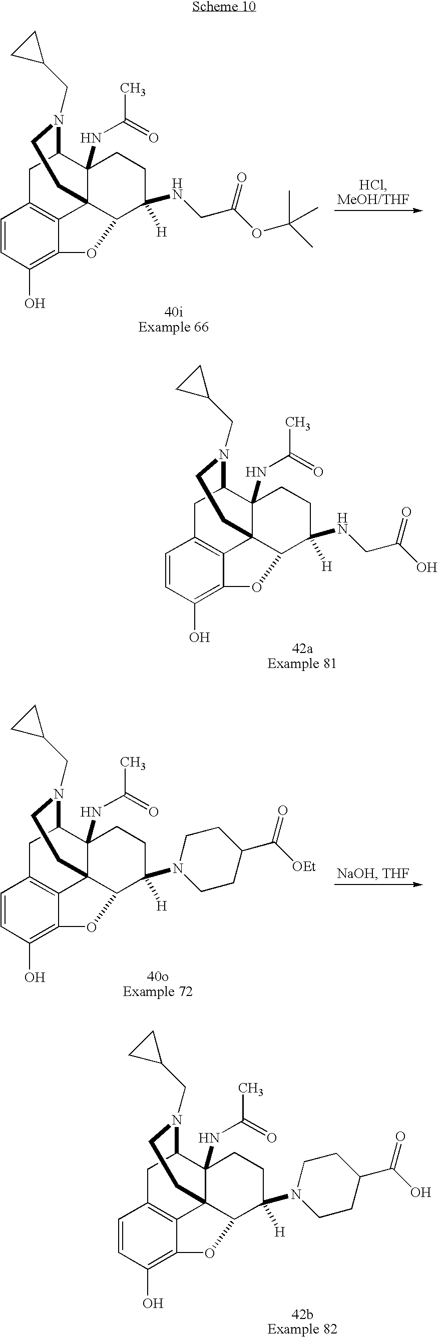

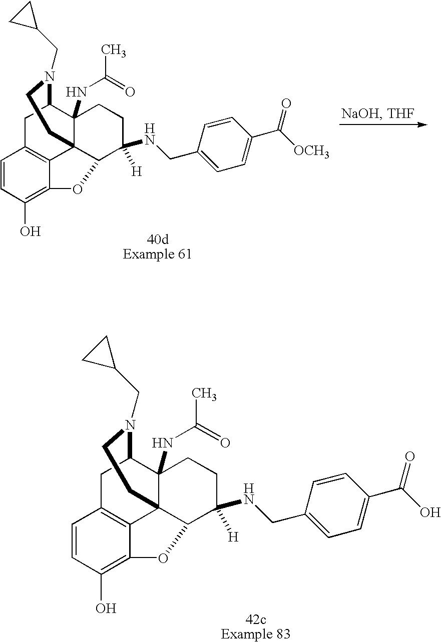

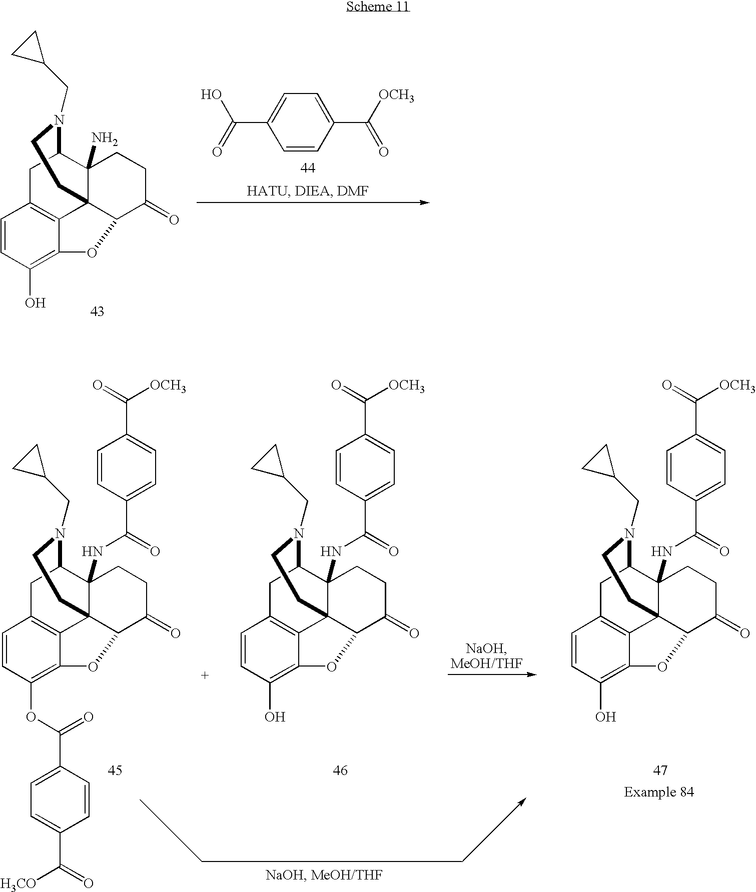

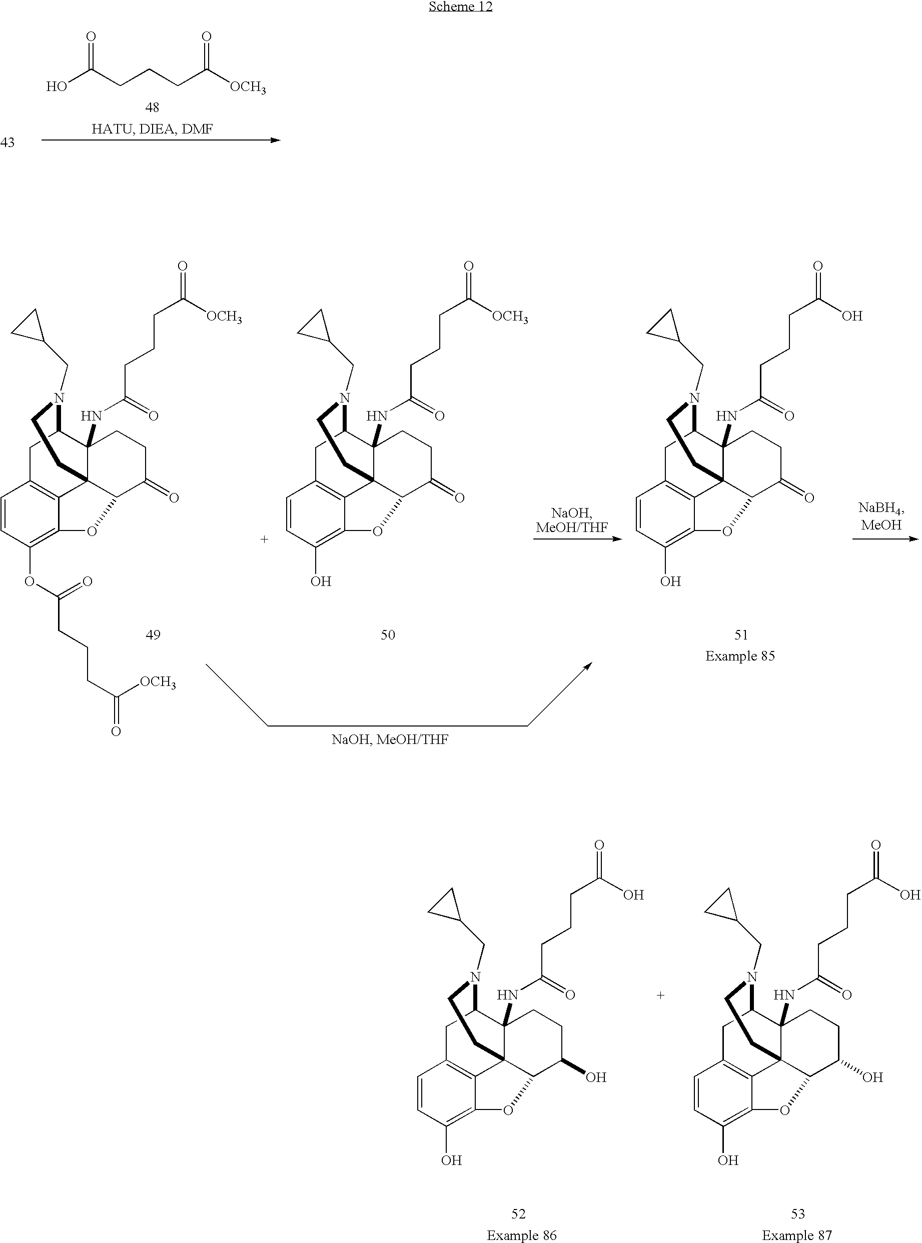

The synthesis of examples 88-117 is described in Scheme 13. N,N-dibenzylnaltrexamine 54 (Sayre, L. M.; Portoghese, P. S. Journal of Organic Chemistry 1980, 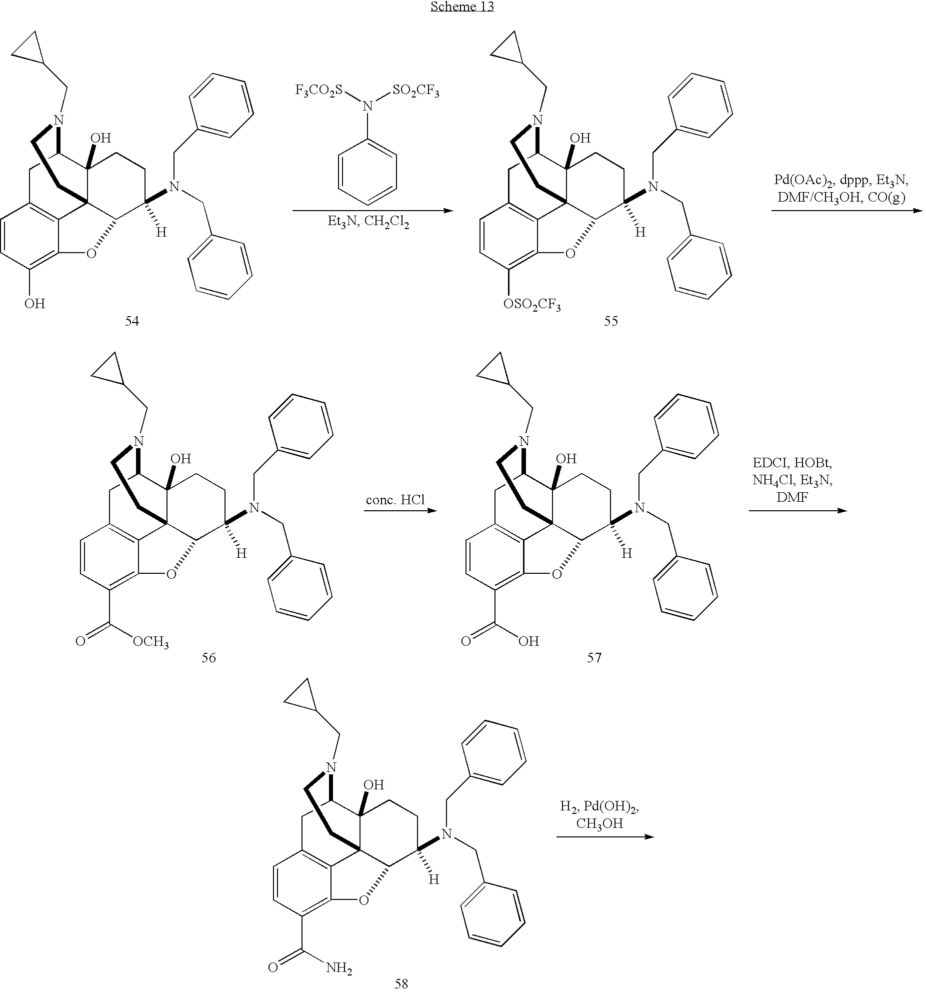 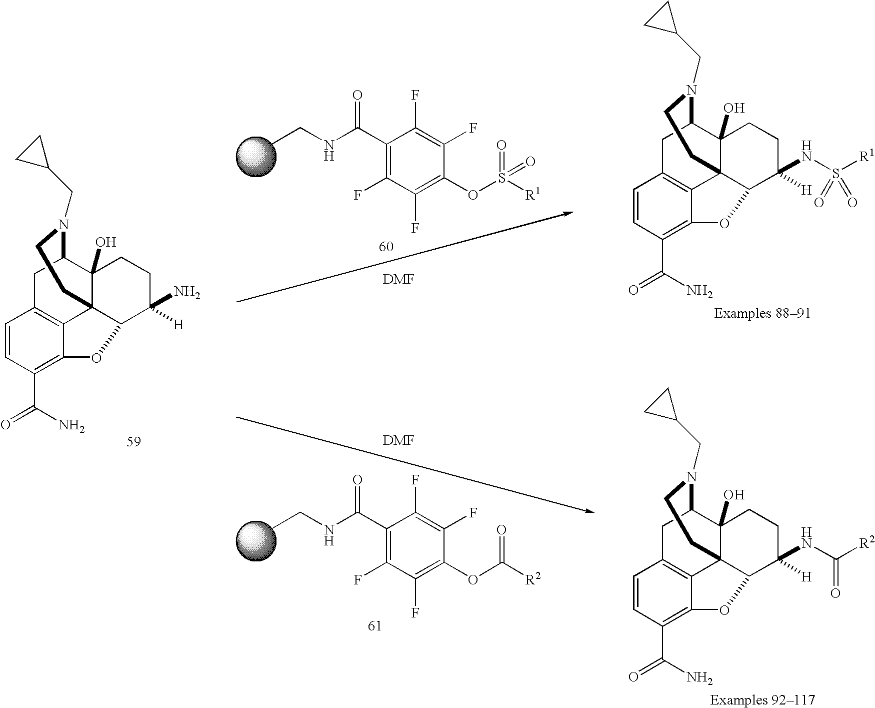
45(16), 3366-8) was converted to the triflate 55 using N-phenyltrifluoromethane sulfonimide in a halogenated solvent and a tertiary amine base. Palladium catalyzed carbonylation of 55 provided the methyl ester 56 which was hydrolyzed under acidic conditions to give the carboxylic acid 57. Coupling of 57 with ammonium chloride using EDCI as coupling agent provided the carboxamide 58. The dibenzyl amine derivative 58 was converted to the primary amine 59 by hydrogenation. Polymeric tetrafluorophenol sulfonates 60 and esters 61 were prepared according to literature procedures (Salvino, J. M., et al., J. Comb. Chem. 2000, 2, 691-697). Condensation of 59 with 60 or 61 provided the corresponding sulfonamides (examples 88-91) or amide derivatives (examples 92-117).

Other compounds having generally similar structures to Naltrexone 1 but differing in the N-substitution on the ring nitrogen (corresponding to the R² substituent in Formula Ia, Ib, Ic, or Id compounds) may be substituted for Naltrexone 1 (in which R² is cyclopropylmethyl) as the starting material for the above schemes. Such compounds include but are not limited to Naloxone, Nalmexone and Oxymorphone. As one skilled in the art will recognize, substitution of these starting materials for Naltrexone in the above schemes may provide additional compounds of Formula Ia, Ib, Ic, or Id differing in the substitution at R² (such as, for Example, allyl, CH₂CH═C(CH₃)₂, and CH₃, when employing Naloxone, Nalmexone and Oxymorphone as starting materials, respectively).

Other features of the invention will become apparent in the course of the following descriptions of exemplary embodiments which are given for illustration of the invention and are not intended to be limiting thereof. The present invention will now be illustrated by reference to the following specific, non-limiting examples. Those skilled in the art of organic synthesis may be aware of still other synthetic routes to the invention compounds. The reagents and intermediates used herein are either commercially available or prepared according to standard literature procedures, unless otherwise described.

### EXAMPLES

Materials: all chemicals were reagent grade and used without further purification. Analytical thin-layer chromatography (TLC) was performed on silica gel thin layer chromatography plates from Alltech and visualized by UV 254 irradiation and iodine. Chromatographic elution solvent systems are reported as volume: volume ratios. All ¹H NMR spectra were recorded at ambient temperature on a Bruker-400 MHz spectrometer. They are reported in ppm on the δ scale, from TMS. LC-MS data were obtained using a Thermo-Finnigan Surveyor HPLC and a Thermo-Finnigan AQA MS using either positive or negative electrospray ionization. Program (positive) Solvent A: 10 mM ammonium acetate, pH 4.5, 1% acetonitrile; solvent B: acetonitrile; column: Michrom Bioresources Magic C18 Macro Bullet, detector: PDA λ=220-300 nm. Gradient: 96% A-100% B in 3.2 minutes, hold 100% B for 0.4 minutes. Program (negative) Solvent A: 1 mM ammonium acetate, pH 4.5, 1% acetonitrile; solvent B: acetonitrile; column: Michrom Bioresources Magic C18 Macro Bullet, detector: PDA λ=220-300 nm. Gradient: 96% A-100% B in 3.2 minutes, hold 100% B for 0.4 minutes.

### Example 1

### Preparation of 4,5α-Epoxy-3,14β-dihydroxy-6-(3,4-dimethylphenyl)-6,7-deshydro-17-(cyclopropylmethyl)morphinan (7a)

A mixture of 5 g (14.6 mmol) of naltrexone 1, 2.31 g (15.3 mmol) of tert-butyldimethylsilyl chloride, and 2.18 g (32.1 mmol) of imidazole in 40 ml of dry DMF was stirred at room temperature overnight under argon. A 2N aqueous solution of Na₂CO₃ was added and the mixture was extracted with diethyl ether. The combined organic extracts were washed with brine and dried over MgSO₄. The mixture was filtered and the filtrate was evaporated under reduced pressure. The crude product was purified by flash chromatography on silica gel; eluent:hexane/Ethyl acetate=8:2. The purification afforded 6.37 g (95%) of 2 obtained as a white solid.

¹H NMR (400 MHz, DMSO-d₆) δ 6.58 (s, 2H), 4.97 (brs, 1H), 4.84 (s, 1H), 3.00 (d, J=4.80 Hz, 1H), 3.00 (d, J=18.40 Hz, 1H), 2.89 (dt, J=4.40 Hz, J=13.6 Hz, 1H), 2.65 (d, J=11.60 Hz, 1H), 2.56 (d, J=5.20 Hz, 1H), 2.43-2.29 (m, 3H), 2.10 (d, J=13.60 Hz, 1H), 1.94 (t, J=11.60 Hz, 1H), 1.77 (d, J=13.20 Hz, 1H), 1.46 (t, J=14.00 Hz, 1H), 1.27 (d, J=12.40 Hz, 1H), 0.94 (s, 9H), 0.91-0.81 (m, 3H), 0.48 (d, J=7.20 Hz, 2H), 0.19 (s, 3H), 0.14 (s, 3H). MS (M+1) 456.2.

To a solution of 2.7 g (5.93 mmol) of compound 2 in 40 ml THF at −78° C. was added dropwise 13.05 ml of a 1N solution of LiHMDS in THF. The reaction mixture was stirred at −78° C. for 2 hrs. A solution of 2.33 g (6.52 mmol) of N-phenylbistrifluoromethanesulphonimide 4 in 10 mL of THF was added to the reaction mixture which was stirred −78° C. for 2 hrs and at 0° C. for 4 hrs. The solution was poured into iced water and extracted with ethyl acetate. The combined organic extracts were washed with water, brine and dried over MgSO₄. The mixture was filtered and the filtrate was evaporated under reduced pressure. The crude product was purified by flash chromatography on silica gel; eluent:hexane/Ethyl acetate=8:2. The purification afforded 1.51 g (43%) of the enol triflate 3 as a white solid.

¹H NMR (400 MHz, DMSO-d₆) δ 6.68 (s, 2H), 6.18 (d, J=6.40 Hz, 1H), 4.86 (s, 1H), 4.90 (brs, 1H), 3.21 (d, J=6.40 Hz, 1H), 3.07 (d, J=18.80 Hz, 1H), 2.71 (d, J=12.4 Hz, 2H), 2.40 (d, J=5.60 Hz, 2H), 2.34-2.03 (m, 4H), 1.49 (d, J=11.60 Hz, 1H), 0.98 (s, 9H), 0.97-0.87 (m, 3H), 0.59-0.47 (m, 2H), 0.07 (s, 6H). MS (M+1) 588.3.

A mixture of the enol triflate 3 (250 mg, 0.425 mmol), 3,4-dimethylphenylboronic acid 5a (0.47 mmol), tetrakis (triphenylphosphine)-palladium(0) (9.8 mg, 0.0085 mmol), lithium chloride (54 mg, 1.275 mmol) and a 2N aqueous solution of sodium carbonate (0.64 ml, 1.275 mmol) in 10 mL of DME was refluxed under N₂ for 3 h. The mixture was cooled to 0° C., water (30 mL) was added, and the mixture was extracted with Ethyl acetate. The combined organic extracts were washed with water, brine and dried over MgSO₄. The mixture was filtered and the filtrate was evaporated under reduced pressure. The crude product 6a was used for the next step without further purification.

A 1N solution of tetrabutylammonium fluoride in THF (0.3 mL) was added to a solution 6a (0.25 mmol) in 5 mL of THF at 0° C. The reaction was stirred at 0° C. for an additional 1 h. After evaporation of solvent, the crude product was purified by flash chromatography on silica gel; eluent:hexane/Ethyl acetate=5:5. The purification provided compound 7a (68%). ¹H NMR (400 MHz, DMSO-d₆) δ 8.86 (brs, 1H), 7.33 (s, 1H), 7.27 (dd, J=9.2 Hz, J=1.64 Hz, 1H), 7.09 (d, J=7.92 Hz, 1H), 6.48 (q, J=7.20 Hz, 2H), 6.17 (dd, J=2.36 Hz, J=3.52 Hz, 1H), 5.41 (s, 1H), 4.68 (brs, 1H), 3.11 (d, J=6.08, 1H), 3.00 (d, J=18.53 Hz, 1H), 2.70-2.54 (m, 2H), 2.36 (d, J=6.16 Hz, 2H), 2.34-2.26 (m, 1H), 2.23 (s, 3H), 2.20 (s, 3H), 2.15-1.99 (m, 2H), 1.49 (d, J=10.8 Hz, 1H), 0.91-0.80 (m, 1H), 0.56-0.44 (m, 2H), 0.18-0.099 (m, 2H). MS (M+1) 430.1.

### Example 2

### Preparation of 4,5α-Epoxy-3,14β-dihydroxy-6-(2-phenoxyphenyl)-6,7-deshydro-17-(cyclopropylmethyl)morphinan

Example 2 was obtained from 3 according to a procedure similar to the one described for the preparation of Example 1.

### Example 3

### Preparation of 4,5α-Epoxy-3,14β-dihydroxy-6-(4-methylsulfonylphenyl)-6,7-deshydro-17-(cyclopropylmethyl)morphinan

Example 3 was obtained from 3 according to a procedure similar to the one described for the preparation of Example 1.

### Example 4

### Preparation of 4,5α-Epoxy-3,14β-dihydroxy-6-(3-trifluoromethylphenyl)-6,7-deshydro-17-(cyclopropylmethyl)morphinan

Example 4 was obtained from 3 according to a procedure similar to the one described for the preparation of Example 1.

### Example 5

### Preparation of 4,5α-Epoxy-3,14β-dihydroxy-6-(3-chlorophenyl)-6,7-deshydro-17-(cyclopropylmethyl)morphinan

Example 5 was obtained from 3 according to a procedure similar to the one described for the preparation of Example 1.

### Example 6

### Preparation of 4,5α-Epoxy-3,14β-dihydroxy-6-(3,5-dichlorophenyl)-6,7-deshydro-17-(cyclopropylmethyl)morphinan

Example 6 was obtained from 3 according to a procedure similar to the one described for the preparation of Example 1.

### Example 7

### Preparation of 4,5α-Epoxy-3,14β-dihydroxy-6-(3-pyridinyl)-6,7-deshydro-17-(cyclopropylmethyl)morphinan

Example 7 was obtained from 3 according to a procedure similar to the one described for the preparation of Example 1.

### Example 8

### Preparation of 4,5α-Epoxy-3,14β-dihydroxy-6-(2-benzo[B]furanyl)-6,7-deshydro-17-(cyclopropylmethyl)morphinan

Example 8 was obtained from 3 according to a procedure similar to the one described for the preparation of Example 1.

### Example 9

### Preparation of 4,5α-Epoxy-3,14β-dihydroxy-6-(4-N,N-diethylaminocarbonylphenyl)-6,7-deshydro-17-(cyclopropylmethyl)morphinan

Example 9 was obtained from 3 according to a procedure similar to the one described for the preparation of Example 1.

### Example 10

### Preparation of 4,5α-Epoxy-3,14β-dihydroxy-6-(4-methoxycarbonylphenyl)-6,7-deshydro-17-(cyclopropylmethyl)morphinan

Example 10 was obtained from 3 according to a procedure similar to the one described for the preparation of Example 1.

### Example 11

### Preparation of 4,5α-Epoxy-3,14β-dihydroxy-6-phenyl-6,7-deshydro-17-(cyclopropylmethyl)morphinan

Example 11 was obtained from 3 according to a procedure similar to the one described for the preparation of Example 1.

### Example 12

### Preparation of 4,5α-Epoxy-3,14β-dihydroxy-6-(4-hydroxyphenyl)-6,7-deshydro-17-(cyclopropylmethyl)morphinan

Example 12 was obtained from 3 according to a procedure similar to the one described for the preparation of Example 1.

### Examples 13 and 14

### Preparation of 4,5α-Epoxy-6-carboxy-6,7-deshydro-3,14β-dihydroxy-17-(cyclopropylmethyl)morphinan (9) and 4,5α-Epoxy-3,14β-dihydroxy-6-(N-phenylethylaminocarbonyl)-6,7-deshydro-17-(cyclopropylmethyl)morphinan (11a)

To a solution of the triflate 3 (1.2 g, 2.04 mmol) in a mixture of methanol (18 ml) and DMF (24 ml) was added triethylamine (412 mg, 4.08 mmol). Carbon monoxide gas was bubbled through the mixture for 5 minutes. To the mixture was added palladium acetate (45.8 mg, 0.204 mmol) followed by DPPF (226.2 mg, 0.408 mmol). The reaction mixture was stirred overnight at 65° C. under carbon monoxide atmosphere. The mixture was cooled to room temperature, poured into water (100 ml) and extracted with Ethyl acetate. The combined organic extracts were washed with brine and dried over MgSO₄. The mixture was filtered and the filtrate was evaporated under reduced pressure. The crude product was purified by flash chromatography on silica gel; eluent:hexane/Ethyl acetate=8:2. After purification, the product 8 (0.85 g, 83%) was obtained as a white solid. ¹H NMR (400 MHz, DMSO-d₆) δ 7.09 (dd, J=2.00 Hz, J=6.00 Hz, 1H), 6.66 (s, 2H), 5.22 (s, 1H), 4.82 (brs, 1H), 3.79 (s, 3H), 3.23 (d, J=6.00 Hz, 1H), 3.22 (d, J=18.8 Hz, 1H), 2.79-2.68 (m, 2H), 2.45 (d, J=6.40 Hz, 2H), 2.38-2.06 (m, 4H), 1.57 (d, J=12.4 Hz, 1H), 0.98 (s, 9H), 0.96-0.92 (m, 1H), 0.63-0.52 (m, 2H), 0.26-0.18 (m, 2H), 0.15 (s, 3H), 0.10 (s, 3H). MS (M+1) 498.1.

To a solution of ester 8 (1.5 g, 3.01 mmol) in a mixture of 25 ml of THF and 10 ml of methanol was added 20 ml of a 1N aqueous solution of NaOH. The mixture was stirred overnight at room temperature. An anhydrous 2N solution of HCl in diethyl ether (10 ml) was added to the mixture which was concentrated to dryness under reduced pressure. A 10% solution of methanol in CH₂Cl₂ (10 mL) was added to the residue. The mixture was filtered and the filtrate was concentrated under vacuum providing 1.06 g (95%) of acid 9 (Example 13) obtained as a white solid. ¹H NMR (400 MHz, DMSO-d₆) δ 9.35 (brs, 1H), 8.95 (brs, 1H), 6.88 (dd, J=2.00 Hz, J=6.00 Hz, 1H), 6.67 (d, J=8.40 Hz, 1H), 6.58 (d, J=8.40 Hz, 1H), 6.47 (s, 1H), 5.30 (s, 1H), 4.06-3.95 (m, 1H), 3.17 (d, J=5.20 Hz, 1H), 3.15-2.98 (m, 2H), 2.98-2.85 (m, 1H), 2.72-2.35 (m, 4H), 2.08 (d, J=19.20 Hz, 1H), 1.68 (d, J=12.4 Hz, 1H), 1.14-0.97 (m, 1H), 0.74-0.54 (m, 2H), 0.53-0.31 (m, 2H). MS (M+1) 370.1.

To a solution of phenylethylamine 10a (0.32 mmol), 141.4 mg (0.32 mmol) of the BOP reagent and 81.8 mg (0.81 mmol) of triethylamine in 5 mL of DMF was added 100 mg of the carboxylic acid 9. The mixture was stirred overnight at room temperature. The mixture was diluted with water (20 mL) and extracted with Ethyl acetate. The combined organic extracts were washed with brine and dried over MgSO₄. The mixture was filtered and the filtrate was evaporated under reduced pressure. The crude product was purified by flash chromatography on silica gel; eluent:hexane/Ethyl acetate=5:5. After purification, the product 11a (Example 14) (80%) was obtained. ¹H NMR (400 MHz, DMSO-d₆) δ 9.03 (s, 1H), 8.01 (s, 1H), 7.33-7.17 (m, 5H), 6.57-6.45 (m, 2H), 5.31 (s, 1H), 4.68 (s, 1H), 3.16-3.05 (m, 1H), 3.04-2.93 (m, 1H), 2.81-2.54 (m, 5H), 2.42-1.92 (m, 7H), 1.46 (d, J=12.00 Hz, 1H), 0.92-0.80 (m, 1H), 0.54-0.43 (m, 2H), 0.16-0.08 (m, 2H). MS (M+1) 473.2.

### Example 15

### Preparation of 4,5α-Epoxy-3,14β-dihydroxy-6-(N-pentanylaminocarbonyl)-6,7-deshydro-17-(cyclopropylmethyl)morphinan

Example 15 was obtained from 9 according to a procedure similar to the one described for the preparation of Example 14.

### Example 16

### Preparation of 4,5α-Epoxy-3,14β-dihydroxy-6-[N-(ethoxyacetyl)aminocarbonyl]-6,7-deshydro-17-(cyclopropylmethyl)morphinan

Example 16 was obtained from 9 according to a procedure similar to the one described for the preparation of Example 14.

### Example 17

### Preparation of 4,5α-Epoxy-3,14β-dihydroxy-6-[N-(tert-butoxypropionyl)aminocarbonyl]-6,7-deshydro-17-(cyclopropylmethyl)morphinan

Example 17 was obtained from 9 according to a procedure similar to the one described for the preparation of Example 14.

### Example 18

### Preparation of 4,5α-Epoxy-3,14β-dihydroxy-6-[N-(methoxybutanoyl)aminocarbonyl]-6,7-deshydro-17-(cyclopropylmethyl)morphinan

Example 18 was obtained from 9 according to a procedure similar to the one described for the preparation of Example 14.

### Example 19

### Preparation of 4,5α-Epoxy-3,14β-dihydroxy-6-[N-(4-morpholinylethyl)aminocarbonyl]-6,7-deshydro-17-(cyclopropylmethyl)morphinan

Example 19 was obtained from 9 according to a procedure similar to the one described for the preparation of Example 14.

### Example 20

### Preparation of 4,5α-Epoxy-3,14β-dihydroxy-6-{N-[1-(4-fluorophenyl)piperazinyl]-aminocarbonyl}-6,7-deshydro-17-(cyclopropylmethyl)morphinan

Example 20 was obtained from 9 according to a procedure similar to the one described for the preparation of Example 14.

### Example 21

### Preparation of 4,5α-Epoxy-3,14β-dihydroxy-6-{4-spiro[3-(2-pyrrolidinone)]piperidinyl}-aminocarbonyl-6,7-deshydro-17-(cyclopropylmethyl)morphinan

Example 21 was obtained from 9 according to a procedure similar to the one described for the preparation of Example 14.

### Example 22

### Preparation of 4,5α-Epoxy-3,14β-dihydroxy-6-{4-spiro[3-(N-methyl-2-pyrrolidinone)]piperidinyl}-aminocarbonyl-6,7-deshydro-17-(cyclopropylmethyl)-morphinan

Example 22 was obtained from 9 according to a procedure similar to the one described for the preparation of Example 14.

### Example 23

### Preparation of 4,5α-Epoxy-3,14β-dihydroxy-6-{N-methyl, N-[1-(S)-isopropyl-2-(3-(S)-hydroxy-pyrrolidin-1″-yl)]ethyl-aminocarbonyl}-6,7-deshydro-17-(cyclopropylmethyl)morphinan

Example 24 was obtained from 9 according to a procedure similar to the one described for the preparation of Example 14.

### Example 24

### Preparation of 4,5α-Epoxy-3,14β-dihydroxy-6-{N-[4R-(3hydroxyphenyl)-3(R),4-dimethyl-1-piperidinyl]aminocarbonyl}-6,7-deshydro-17-(cyclopropylmethyl)morphinan

Example 24 was obtained from 9 according to a procedure similar to the one described for the preparation of Example 14.

### Example 25

### Preparation of 4,5α-Epoxy-3,14β-dihydroxy-6-[N-3-(R)-phenyl(ethoxypropionyl)aminocarbonyl]-6,7-deshydro-17-(cyclopropylmethyl)morphinan

Example 25 was obtained from 9 according to a procedure similar to the one described for the preparation of Example 14.

### Example 26

### Preparation of 4,5α-Epoxy-3,14β-dihydroxy-6-[N-(methoxyacetyl)aminocarbonyl]-6,7-deshydro-17-(cyclopropylmethyl)morphinan

Example 26 was obtained from 9 according to a procedure similar to the one described for the preparation of Example 14.

### Examples 27 and 28

### Preparation of 4,5α-Epoxy-6-methoxycarbonyl-6,7-deshydro-3,14β-dihydroxy-17-(cyclopropylmethyl)morphinan (12) and 4,5α-Epoxy-6(α/β)-methoxycarbonyl-3,14β-dihydroxy-17-(cyclopropylmethyl)morphinan (13)

To a solution of 100 mg (0.26 mmol) of compound 8 in 5 mL of THF at 0° C. was added 0.26 mL of a 1N solution of TBAF in THF. The reaction mixture was stirred at 0° C. for 1 h. The mixture was then concentrated under reduced pressure. The crude product was purified by flash chromatography on silica gel; eluent:hexane/Ethyl acetate=7:3. After purification, the product 12 (Example 27) (82 mg, 82%) was obtained as a white solid. MS (M+1) 384.1.

A mixture of 75 mg (0.196 mmol) of compound 12, 7.5 mg of 10% palladium on carbon in 10 mL of Ethyl acetate was stirred at room temperature under H₂ at atmospheric pressure for 12 h. The mixture was filtered through celite. The filtrate was concentrated under reduced pressure and the crude compound was purified by HPLC. After purification, 62 mg of compound 13 (Example 28) was obtained as a white solid. MS (M+1) 386.1.

### Example 29

### Preparation of 4,5α-Epoxy-6(α/β)-carboxy-3,14β-dihydroxy-17-(cyclopropylmethyl)morphinan (14)

A mixture of 100 mg (0.27 mmol) of 2, 10 mg of 10% palladium on carbon in 10 mL of methanol was stirred at room temperature under H₂ at atmospheric pressure for 12 h. The mixture was filtered through celite. The filtrate was concentrated under reduced pressure. The desired product 14 (85 mg, 84.8%) was obtained as a white solid. MS (M+1) 372.1.

### Examples 30 and Example 31

### Preparation of 4,5α-Epoxy-3,14β-dihydroxy-6,6Z(methoxycarbonylethenyl)-17-(cyclopropylmethyl)morphinan (16) (Example 30) and 4,5α-Epoxy-3,14β-dihydroxy-6,6E(methoxycarbonylethenyl)-17-(cyclopropylmethyl)morphinan (17) (Example 31)

To a solution of 1.41 g (35.2 mmol) of NaH in 200 mL of DME was added 7.4 g (35.2 mmol) of methyl diethylphosphonoacetate 15 in 50 mL of DME at 0° C. The mixture was warmed slowly to room temperature and stirring was continued for an additional 30 min at room temperature. The mixture was cooled to 0° C. and 10 g (29.3 mmol) of naltrexone 1 was added. The mixture was heated to reflux for 16 h. The mixture was cooled to 0° C.; 200 mL of H₂O was added to the mixture which was extracted with Ethyl acetate. The combined organic extracts were washed with brine and dried over MgSO₄. The mixture was filtered and the filtrate was evaporated under reduced pressure. The crude product was purified by flash chromatography on silica gel; eluent:hexane/Ethyl acetate=7:3. After purification, 105 mg of compound 16, 559 mg of compound 17 and 185 mg of mixture of compounds 16 and 17 were obtained (total yield: 73%).

16 (Example 30) ¹H NMR (400 MHz, DMSO-d₆) δ 9.15 (s, 1H), 6.55 (d, J=8.08 Hz, 1H), 6.50 (d, J=8.08 Hz, 1H), 6.16 (t, J=1.60 Hz, 1H), 4.97 (s, 2H), 3.56 (s, 3H), 3.53 (t, J=3.20 Hz, 1H), 3.03 (d, J=5.20 Hz, 1H), 2.94 (d, J=18.8 Hz, 1H), 2.64 (dd, J=4.40, J=11.2 Hz, 1H), 2.46 (d, J=5.20 Hz, 1H), 2.40-2.21 (m, 3H), 2.05-1.94 (m, 2H), 1.59 (dt, J=3.60, J=12.80 Hz, 1H), 1.35-1.21 (m, 1H), 1.21-1.11 (m, 1H), 0.90-0.79 (m, 1H), 0.53-0.42 (m, 2H), 0.17-0.06 (m, 2H). MS (M+1) 398.2.

17 (Example 31) ¹H NMR (400 MHz, DMSO-d₆) δ 8.93 (s, 1H), 6.51 (d, J=9.60 Hz, 1H), 6.46 (d, J=8.00 Hz, 1H), 5.61 (d, J=4.00 Hz, 1H), 4.79 (s, 1H), 4.66 (s, 1H), 3.62 (s, 3H), 3.18 (d, J=15.60 Hz, 1H), 2.66-2.53 (m, 2H), 2.39-2.28 (m, 2H), 2.22-2.01 (m, 2H), 1.98-1.81 (m, 2H), 1.38 (d, J=11.2 Hz, 1H), 0.91-0.79 (m, 1H), 0.53-0.42 (m, 2H), 0.16-0.07 (m, 2H). MS (M+1) 398.2.

### Example 33

### Preparation of 4,5α-Epoxy-3,14β-dihydroxy-6,6Z(N-acryloylamino-acetic acid)-17-(cyclopropylmethyl)morphinan (21a)

To a solution of 1 g (2.52 mmol) of the carboxylic acid 16 in 50 mL of THF and 40 mL of methanol was added 10 mL of a 1N aqueous solution of NaOH. The mixture was stirred overnight at room temperature. An anhydrous 2N solution of HCl in diethyl ether (10 ml) was added to the mixture which was concentrated to dryness under reduced pressure. A 10% solution of methanol in CH₂Cl₂ (200 mL) was added to the residue. The mixture was filtered and the filtrate was concentrated under vacuum providing 0.97 g (100%) of acid 18 obtained as a white solid. MS (M+1) 384.1.

To a solution of the amine 10m (0.62 mmol), the BOP reagent (274 mg, 0.62 mmol) and Et₃N (157.6 mg, 1.56 mmol) in 5 mL of DMF was added 200 mg (0.52 mmol) of acid 18. The mixture was stirred at room temperature for 16 h. The mixture was diluted with water and extracted with Ethyl acetate. The combined organic extracts were washed with brine, with an aqueous saturated solution of sodium bicarbonate and dried over MgSO₄. The mixture was filtered and the filtrate was evaporated under reduced pressure. The crude product was purified by flash chromatography on silica gel; eluent: hexane/Ethyl acetate system of increasing polarity. After purification, the product 20a (example 32) (86%) was obtained. ¹H NMR (400 MHz, DMSO-d₆) δ 8.96 (brs, 1H, D₂O exchangeable), 8.25 (t, J=4.40 Hz, D₂O exchangeable), 6.43 (d, J=8.40 Hz, 1H), 6.37 (d, J=8.40 Hz, 1H), 6.08 (s, 1H), 4.81 (brs, 1H, D₂O exchangeable), 4.76 (s, 1H), 3.66 (dd, J=6.0 Hz, J=1.47 Hz, 2H), 3.53 (dt, J=14.4 Hz, J=3.60 Hz, 1H), 3.48 (s, 3H), 2.89 (d, J=5.20 Hz, 1H), 2.82 (d, J=18.4 Hz, 1H), 2.51 (dd, J=11.6 Hz, J=4.40 Hz, 1H), 2.28-2.05 (m, 4H), 1.90-1.82 (m, 2H), 1.39 (dt, J=12.8 Hz, J=3.2 Hz, 1H), 1.20-1.13 (m, 1H), 1.10-1.00 (m, 1H), 0.78-0.66 (m, 1H), 0.41-0.30 (m, 2H), 0.04-0 (m, 2H). MS (M+1) 455.1.

To a solution of the previously obtained ester 20a in a 1:1 mixture methanol/THF (20 mL) was added a 1N aqueous solution of NaOH (3 equiv.). The reaction mixture was stirred for 12 h at room temperature. An anhydrous 2N solution of HCl in diethyl ether (10 ml) was added to the mixture which was concentrated to dryness under reduced pressure. A 10% solution of methanol in CH₂Cl₂ (200 mL) was added to the residue. The mixture was filtered and the filtrate was concentrated under vacuum providing the carboxylic acid 21a obtained as a white solid.

21a (Example 33) ¹H NMR (400 MHz, DMSO-d₆) δ 9.38 (brs, 1H, D₂O exchangeable), 8.34 (t, J=6.80 Hz, 1H, D₂O exchangeable), 6.70 (d, J=8.0 Hz, 1H), 6.57 (d, J=8.0 Hz, 1H), 6.26 (s, 1H), 5.05 (s, 1H), 3.83 (d, J=5.60, 1H), 3.67 (s, 3H), 3.31-3.18 (m, 2H), 3.10-2.93 (m, 2H), 2.88-2.76 (m, 1H), 2.59-2.42 (m, 4H), 2.32-2.18 (m, 1H), 1.79-1.68 (m, 1H), 1.47 (d, J=9.60 Hz, 1H), 1.28-1.16 (m, 1H), 1.08-0.96 (m, 1H), 0.75-0.54 (m, 2H), 0.52-0.33 (m, 2H). MS (M+1) 441.0.

### Example 34

### Preparation of 4,5α-Epoxy-3,14β-dihydroxy-6,6Z(N-acryloyl-piperidine-2(R/S)-carboxylic acid ethyl ester)-17-(cyclopropylmethyl)morphinan

Example 34 was obtained from 18 according to a procedure similar to the one described for the preparation of Example 32 and Example 33.

### Example 35

### Preparation of 4,5α-Epoxy-3,14β-dihydroxy-6,6Z(N-acryloyl-piperidine-2(R/S)-carboxylic acid methyl ester)-17-(cyclopropylmethyl)morphinan

Example 35 was obtained from 18 according to a procedure similar to the one described for the preparation of Example 32 and Example 33.

### Example 36

### Preparation of 4,5α-Epoxy-3,14β-dihydroxy-6,6Z(N-acryloyl-piperidine-3(R/S)-carboxylic acid ethyl ester)-17-(cyclopropylmethyl)morphinan

Example 36 was obtained from 18 according to a procedure similar to the one described for the preparation of Example 32 and Example 33.

### Example 37

### Preparation of 4,5α-Epoxy-3,14β-dihydroxy-6,6Z(N-acryloyl-piperidine-3(R/S)-carboxylic acid)-17-(cyclopropylmethyl)morphinan

Example 37 was obtained from 18 according to a procedure similar to the one described for the preparation of Example 32 and Example 33.

### Example 38

### Preparation of 4,5α-Epoxy-3,14β-dihydroxy-6,6E(carboxyethenyl)-17-(cyclopropylmethyl)morphinan

Example 38 was obtained from 17 according to a procedure similar to the one described for the preparation of 18 from 16.

### Example 39

### Preparation of 4,5α-Epoxy-3,14β-dihydroxy-6,6E(N-acryloylamino-acetic acid methyl ester)-17-(cyclopropylmethyl)morphinan

Example 39 was obtained from 22 (example 38) according to a procedure similar to the one described for the preparation of Example 32 and Example 33.

### Example 40

### Preparation of 4,5α-Epoxy-3,14β-dihydroxy-6,6E(N-acryloylamino-acetic acid)-17-(cyclopropylmethyl)morphinan

Example 40 was obtained from 22 (example 38) according to a procedure similar to the one described for the preparation of Example 32 and Example 33.

### Example 41

### Preparation of 4,5α-Epoxy-3,14β-dihydroxy-6,6E(N-acryloylamino-butyric acid methyl ester)-17-(cyclopropylmethyl)morphinan

Example 41 was obtained from 22 (example 38) according to a procedure similar to the one described for the preparation of Example 32 and Example 33.

### Example 42

### Preparation of 4,5α-Epoxy-3,14β-dihydroxy-6,6E(N-acryloylamino-butyric acid)-17-(cyclopropylmethyl)morphinan

Example 42 was obtained from 22 (example 38) according to a procedure similar to the one described for the preparation of Example 32 and Example 33.

### Example 43

### Preparation of 4,5α-Epoxy-3,14β-dihydroxy-6,6E(N-acryloyl-piperidine-2(R/S)-carboxylic acid ethyl ester)-17-(cyclopropylmethyl)morphinan

Example 43 was obtained from 22 (example 38) according to a procedure similar to the one described for the preparation of Example 32 and Example 33.

### Example 44

### Preparation of 4,5α-Epoxy-3,14β-dihydroxy-6,6E(N-acryloyl-piperidine-2(R/S)-carboxylic acid)-17-(cyclopropylmethyl)morphinan

Example 44 was obtained from 22 (example 38) according to a procedure similar to the one described for the preparation of Example 32 and Example 33.

### Example 45

### Preparation of 4,5α-Epoxy-3,14β-dihydroxy-6,6E(N-acryloyl-piperidine-3(R/S)-carboxylic acid ethyl ester)-17-(cyclopropylmethyl)morphinan

Example 45 was obtained from 22 (example 38) according to a procedure similar to the one described for the preparation of Example 32 and Example 33.

### Example 46

### Preparation of 4,5α-Epoxy-3,14β-dihydroxy-6,6E(N-acryloyl-piperidine-3(R/S)-carboxylic acid)-17-(cyclopropylmethyl)morphinan

Example 46 was obtained from 22 (example 38) according to a procedure similar to the one described for the preparation of Example 32 and Example 33.

### Example 47

### Preparation of 4,5α-Epoxy-3,14β-dihydroxy-6,6E(N-acryloyl-piperidine-4-carboxylic acid ethyl ester)-17-(cyclopropylmethyl)morphinan

Example 47 was obtained from 22 (example 38) according to a procedure similar to the one described for the preparation of Example 32 and Example 33.

### Example 48

### Preparation of 4,5α-Epoxy-3,14β-dihydroxy-6,6E(N-acryloyl-piperidine-4-carboxylic acid)-17-(cyclopropylmethyl)morphinan

Example 48 was obtained from 22 (example 38) according to a procedure similar to the one described for the preparation of Example 32 and Example 33.

### Example 49

### Preparation of 4,5α-Epoxy-3,14β-dihydroxy-6,6E(N-acryloyl-piperidine-4-carboxylic acid)-17-(cyclopropylmethyl)morphinan

Example 49 was obtained from 22 (example 38) according to a procedure similar to the one described for the preparation of Example 32.

### Example 50

### Preparation of 4,5α-Epoxy-3,14β-dihydroxy-6,6E[N-(4-hydroxymethyl-piperidin-1 yl)-propenone]-17-(cyclopropylmethyl)morphinan

Example 50 was obtained from 22 (example 38) according to a procedure similar to the one described for the preparation of Example 32.

### Example 51

### Preparation of 4,5α-Epoxy-3,14β-dihydroxy-6,6E(N-acryloyl-piperidine-3(R/S)-carboxamide)-17-(cyclopropylmethyl)morphinan

Example 51 was obtained from 22 (example 38) according to a procedure similar to the one described for the preparation of Example 32.

### Example 52

### Preparation of 4,5α-Epoxy-3,14β-dihydroxy-6,6E(N-acryloyl-piperidine-4-carboxamide)-17-(cyclopropylmethyl)morphinan

Example 52 was obtained from 22 (Example 38) according to a procedure similar to the one described for the preparation of Example 32.

### Example 53

### Preparation of 4,5α-Epoxy-3,14β-dihydroxy-6,6E(N-acryloyl-piperidine-4-carboxamide)-17-(cyclopropylmethyl)morphinan

Example 53 was obtained from 22 (Example 38) according to a procedure similar to the one described for the preparation of Example 32.

### Example 54

### Preparation of 4,5α-Epoxy-3,14β-dihydroxy-6(R/S)-(acetylamino-acetic acid methyl ester)-17-(cyclopropylmethyl)morphinan (28)

To a solution of 2 g (5 mmol) of a mixture of esters 16 and 17 in 20 mL of THF and 8 mL of methanol was added 20 mL of a 1N aqueous solution of NaOH. The mixture was stirred for 2 h at room temperature. An anhydrous 2N solution of HCl in diethyl ether (10 ml) was added to the mixture which was concentrated to dryness under reduced pressure. A 10% solution of methanol in CH₂Cl₂ (200 mL) was added to the residue. The mixture was filtered and the filtrate was concentrated under vacuum providing the carboxylic acids 18/22 used for the next step without further purification.

To a solution of the amine 10m (534 mg, 6 mmol), the BOP reagent (2.65, 6 mmol) and Et₃N (1.52 g, 15 mmol) in 10 mL of DMF was added 5 mmol of the carboxylic acids 18/22. The mixture was stirred at room temperature for 16 h. The mixture was diluted with water and extracted with Ethyl acetate. The combined organic extracts were washed with brine, with an aqueous saturated solution of sodium bicarbonate and dried over MgSO₄. The mixture was filtered and the filtrate was evaporated under reduced pressure. The crude product was purified by flash chromatography on silica gel; eluent: hexane/Ethyl acetate system of increasing polarity. After purification, 1.87 g (82%) of the mixture 20a/24a was obtained.

A mixture of 500 mg (1.1 mmol) of the mixture 20a/24a, 50 mg of 10% palladium on carbon in 10 ml of dichloromethane and 10 mL of methanol was stirred at room temperature under H₂ at atmospheric pressure for 1 h. The mixture was filtered through celite. The filtrate was concentrated under reduced pressure. The crude product was purified by flash chromatography on silica gel; eluent:CH₂Cl₂/methanol=8:2. After purification, the product 28 (example 54) (358 mg, 71.3%) was obtained as a white solid. MS (M+1) 457.0.

### Example 55

### Preparation of 4,5α-Epoxy-3,14β-dihydroxy-6,6Z(hydroxyethylidene)-17-(cyclopropylmethyl)morphinan (32)

To a suspension of 572 mg (14.30 mmol) of NaH in 100 mL of DME at 0° C. was added dropwise 3 g (14.30 mmol) of methyl diethylphosphonoacetate 15. The mixture was stirred at room temperature for 30 minutes. The mixture was then cooled (ice bath) and a solution of 5.43 g (11.92 mmol) of compound 2 in 10 mL of DME was added. The mixture was heated to reflux for 12 h. The mixture was then cooled (ice bath); 100 mL of water and 100 mL of Ethyl acetate were added to the solution. The aqueous phase was further extracted with Ethyl acetate. The combined organic extracts were washed with water, brine and dried over MgSO₄. The mixture was filtered and the filtrate was concentrated under reduced pressure. The crude product was purified by flash chromatography on silica gel; eluent:hexane/Ethyl acetate=7:3. After purification, the products 29 (2.6 g) and 30 (1.3 g) were obtained (total yield: 83.6%).

29: MS (M+1) 512.2.

30: MS (M+1) 512.2.

A 1N solution of diisobutylaluminum hydride in toluene (14.94 ml, 14.94 mmol), was added dropwise under N₂ to a solution of 2.55 g (4.98 mmol) of compound 29 in 40 mL of toluene at −78° C. The reaction was stirred for 2 h at −78° C. The reaction was then quenched by addition of 5 mL of an aqueous solution of ammonium chloride. The mixture was warmed to room temperature and filtered through a short column of silica gel. The filtrate was concentrated and the residue was further purified by flash chromatography (eluent:Ethyl acetate/Hexane=5:5). After purification, the desired product 31 (1.49 g, 61.85%) was obtained as a white solid. MS (M+1) 484.3.

To a solution of 200 mg (0.41 mmol) of compound 31 in 5 mL of THF at 0° C. was added 0.49 mL (0.49 mmol) of a 1N solution of TBAF in THF. The mixture was stirred at 0° C. for 1 h. The mixture was concentrated and the residue was purified by flash chromatography (eluent:Ethyl acetate/Hexane=5:5). After purification, the desired product 32 (example 55) (144 mg, 95%) was obtained as a white solid. MS (M+1) 370.0.

### Example 56

### Preparation of 4,5α-Epoxy-3,14β-dihydroxy-6,6E(hydroxyethylidene)-17-(cyclopropylmethyl)morphinan

Example 56 was obtained from 30 according to a procedure similar to the one described for the preparation of Example 55 from 29.

### Example 57

### Preparation of 4,5α-Epoxy-3,14β-dihydroxy-6-ethylene-6,7-deshydro-17-(cyclopropylmethyl)morphinan (37)

To a solution of 200 mg (0.34 mmol) of the triflate 3 in 15 mL of THF was added 129.4 mg (0.408 mmol) of vinyltributyltin 35, 43.2 mg (1.02 mmol) of lithium chloride and 7.86 mg (0.0068 mmol) of tetrakis(triphenylphosphine)palladium (0). The resulting mixture was heated at reflux for 17 h under nitrogen and cooled to room temperature. The mixture was concentrated under reduced pressure. The crude residue was purified by flash chromatography (eluent:Ethyl acetate/Hexane=7:3). After purification, the desired product 36 (98 mg, 63%) was obtained as a white solid. MS (M+1) 466.2.

To a solution of 98 mg (0.20 mmol) of compound 36 in 5 mL of THF at 0° C. was added 0.24 mL of a 1N solution of TBAF in THF. The mixture was stirred at 0° C. for 1 h. The mixture was concentrated and the residue was purified by flash chromatography (eluent:Ethyl acetate/Hexane=3:7). After purification, the desired product 37 (Example 57) (68 mg, 97%) was obtained as a white solid. MS (M+1) 352.

### Examples 58 and 73

### Preparation of 4,5α-Epoxy-3-hydroxy-14β-acetylamino-6-β-benzylamino-17-(cyclopropylmethyl)morphinan (40a) (Example 58) and 4,5α-Epoxy-3-hydroxy-14β-acetylamino-6-α-benzylamino-17-(cyclopropylmethyl)morphinan (41a) (Example 73)

To a solution of the ketone 38 (50 mg, 0.131 mmol, 1 eq.) in anhydrous methanol (2 mL) was added dropwise a solution of benzylamine 39a (70 mg, 0.654 mmol, 5 eq.) in anhydrous methanol (1 mL). The resulting solution was stirred at room temperature for 4 h. Sodium cyanoborohydride (25 mg, 0.392 mmol, 3 eq.) was then added to the mixture, which was allowed to stir for an additional 16 h at room temperature. The reaction mixture was acidified to pH 5 with glacial acetic acid and concentrated in vacuo. The crude product was purified by preparative HPLC [Genesis C18 column (Jones Chromatography), eluent: 5-50% acetonitrile in water (+0.1% TFA). Two fractions were isolated: Fraction 1: t_{R}=1.38 min, 15.5 mg corresponding to the α-isomer, 41a (Example 73); [M+H]⁺ 474; Fraction 2: t_{R}=1.51 min, 45.6 mg corresponding to the β-isomer, 40a (Example 58); [M+H]⁺ 474; total yield (combined fraction 1 and 2): 68%.

### Example 59

### Preparation of 4,5α-Epoxy-3-hydroxy-14β-acetylamino-6-β-(4-chlorobenzylamino)-17-(cyclopropylmethyl)morphinan

Example 59 was obtained from 38 according to a procedure similar to the one described for the preparation of Example 58 and Example 73.

### Example 60

### Preparation of 4,5α-Epoxy-3-hydroxy-14β-acetylamino-6-β-(c-pyridinyl-3-yl-methylamino)-17-(cyclopropylmethyl)morphinan

Example 60 was obtained from 38 according to a procedure similar to the one described for the preparation of Example 58 and Example 73.

### Example 61

### Preparation of 4,5α-Epoxy-3-hydroxy-14β-acetylamino-6-β-[(4-(methyloxycarbonyl)-benzylamino]-17-(cyclopropylmethyl)morphinan

Example 61 was obtained from 38 according to a procedure similar to the one described for the preparation of Example 58 and Example 73.

### Example 62

### Preparation of 4,5α-Epoxy-3-hydroxy-14β-acetylamino-6-β-[3,4-dichlorobenzylamino]-17-(cyclopropylmethyl)morphinan

Example 62 was obtained from 38 according to a procedure similar to the one described for the preparation of Example 58 and Example 73.

### Example 63

### Preparation of 4,5α-Epoxy-3-hydroxy-14β-acetylamino-6-β-[(2-tert-butyloxycarbonyl,2-(R)propyl)methylamino]-17-(cyclopropylmethyl)morphinan

Example 63 was obtained from 38 according to a procedure similar to the one described for the preparation of Example 58 and Example 73.

### Example 64

### Preparation of 4,5α-Epoxy-3-hydroxy-14β-acetylamino-6-β-(phenylamino)-17-(cyclopropylmethyl)morphinan

Example 64 was obtained from 38 according to a procedure similar to the one described for the preparation of Example 58 and Example 73.

### Example 65

### Preparation of 4,5α-Epoxy-3-hydroxy-14β-acetylamino-6-β-(phenylamino)-17-(cyclopropylmethyl)morphinan

Example 65 was obtained from 38 according to a procedure similar to the one described for the preparation of Example 58 and Example 73.

### Example 66

### Preparation of 4,5α-Epoxy-3-hydroxy-14β-acetylamino-6-β-(tert-butyloxycarbonyl-methylamino)-17-(cyclopropylmethyl)morphinan

Example 66 was obtained from 38 according to a procedure similar to the one described for the preparation of Example 58 and Example 73.

### Example 67

### Preparation of 4,5α-Epoxy-3-hydroxy-14β-acetylamino-6-β-[2-(3,4-dimethoxyphenyl)-ethylamino]-17-(cyclopropylmethyl)morphinan

Example 67 was obtained from 38 according to a procedure similar to the one described for the preparation of Example 58 and Example 73.

### Example 68

### Preparation of 4,5α-Epoxy-3-hydroxy-14β-acetylamino-6-β-(2-molpholinyl-4-yl-ethylamino]-17-(cyclopropylmethyl)morphinan

Example 68 was obtained from 38 according to a procedure similar to the one described for the preparation of Example 58 and Example 73.

### Example 69

### Preparation of 4,5α-Epoxy-3-hydroxy-14β-acetylamino-6β-piperidino-17-(cyclopropylmethyl)morphinan

Example 69 was obtained from 38 according to a procedure similar to the one described for the preparation of Example 58 and Example 73.

### Example 70

### Preparation of 4,5α-Epoxy-3-hydroxy-14β-acetylamino-6 β-morpholino-17-(cyclopropylmethyl)morphinan

Example 70 was obtained from 38 according to a procedure similar to the one described for the preparation of Example 58 and Example 73.

### Example 71

### Preparation of 4,5α-Epoxy-3-hydroxy-14β-acetylamino-6 β-N-benzyl-N-methylamino-17-(cyclopropylmethyl)morphinan

Example 71 was obtained from 38 according to a procedure similar to the one described for the preparation of Example 58 and Example 73.

### Example 72

### Preparation of 4,5α-Epoxy-3-hydroxy-14β-acetylamino-6 β-[(4-ethyloxycarbonyl)piperidino]-17-(cyclopropylmethyl)morphinan

Example 72 was obtained from 38 according to a procedure similar to the one described for the preparation of Example 58 and Example 73.

### Example 74

### Preparation of 4,5α-Epoxy-3-hydroxy-14β-acetylamino-6-α-(4-chlorobenzylamino)-17-(cyclopropylmethyl)morphinan

Example 74 was obtained from 38 according to a procedure similar to the one described for the preparation of Example 58 and Example 73.

### Example 75

### Preparation of 4,5α-Epoxy-3-hydroxy-14β-acetylamino-6-α-[3,4-dichlorobenzylamino]-17-(cyclopropylmethyl)morphinan

Example 75 was obtained from 38 according to a procedure similar to the one described for the preparation of Example 58 and Example 73.

### Example 76

### Preparation of 4,5α-Epoxy-3-hydroxy-14β-acetylamino-6-α-[(2-tert-butyloxycarbonyl,2-(R)propyl)methylamino]-17-(cyclopropylmethyl)morphinan

Example 76 was obtained from 38 according to a procedure similar to the one described for the preparation of Example 58 and Example 73.

### Example 77

### Preparation of 4,5α-Epoxy-3-hydroxy-14β-acetylamino-6-α-[(2-tert-butyloxycarbonyl,2-(R)propyl)methylamino]-17-(cyclopropylmethyl)morphinan

Example 77 was obtained from 38 according to a procedure similar to the one described for the preparation of Example 58 and Example 73.

### Example 78

### Preparation of 4,5α-Epoxy-3-hydroxy-14β-acetylamino-6-α-(butylamino)-17-(cyclopropylmethyl)morphinan

Example 78 was obtained from 38 according to a procedure similar to the one described for the preparation of Example 58 and Example 73.

### Example 79

### Preparation of 4,5α-Epoxy-3-hydroxy-14β-acetylamino-6-α-(tert-butyloxycarbonyl-methylamino)-17-(cyclopropylmethyl)morphinan

Example 79 was obtained from 38 according to a procedure similar to the one described for the preparation of Example 58 and Example 73.

### Example 80

### Preparation of 4,5α-Epoxy-3-hydroxy-14β-acetylamino-6-α-[2-(3,4-dimethoxyphenyl)-ethylamino]-17-(cyclopropylmethyl)morphinan

Example 80 was obtained from 38 according to a procedure similar to the one described for the preparation of Example 58 and Example 73.

### Example 81

### Preparation of 4,5α-Epoxy-3-hydroxy-14β-acetylamino-6 β-carboxymethylamino-17-(cyclopropylmethyl)morphinan (42a)

To a stirred solution of 40i (25 mg) in tetrahydrofuran (900 μL) was added a solution of a concentrated solution of HCl (100 μL). The solution was stirred at room temperature for 16 hours. The mixture was concentrated in vacuo. The crude product was purified by preparative HPLC [Genesis C18 column (Jones Chromatography), eluent: 2-20% acetonitrile in water (+0.1% TFA), t_{R}=0.96 min; 42a (11 mg, 30%); [M+H]⁺ 442.

### Example 82

### Preparation of 4,5α-Epoxy-3-hydroxy-14β-acetylamino-6 β-(4-carboxypiperidino)-17-(cyclopropylmethyl)morphinan (42a)

To a stirred solution of 40o (15 mg) in a mixture tetrahydrofuran/methanol (1:1) (1 mL) was added a 1N aqueous solution of NaOH (100 μL). The solution was stirred at room temperature for 16 h. The reaction mixture was acidified to pH 5 with glacial acetic acid and concentrated in vacuo. The crude product was purified by preparative HPLC [Genesis C18 column (Jones Chromatography), eluent: 5-20% acetonitrile in water (+0.1% TFA), t_{R}=0.85 min; 42b (3 mg, 30%); [M+H]⁺ 496.

### Example 83

### Preparation of 4,5α-Epoxy-3-hydroxy-14β-acetylamino-6 β-(4-carboxybenzylamino)-17-(cyclopropylmethyl)morphinan

Example 83 was obtained from 40d (example 61) according to a procedure similar to the one described for the preparation of Example 82 from 40o (example 72).

### Example 84

### Preparation of 4,5α-Epoxy-3-hydroxy-14β-[(4-carboxy)phenylcarbonylamino]-6-keto-17-(cyclopropylmethyl)morphinan (47)

To a cold (0° C.) solution of the hydrochloric acid salt of 43 (0.430 g, 1.14 mmol) and diisopropylethylamine (1.2 mL, 6.8 mmol) in anhydrous dimethylformamide (10 mL) was added mono-methylterephthalate (0.265 g, 1.47 mmol) followed by a solution of HATU (0.559 g, 1.47 mmol) in anhydrous dimethylformamide (2 mL). The mixture was stirred for 2 h at 0° C. and stirring was continued at room temperature for an additional 16 h. The mixture was concentrated in vacuo. Ethyl acetate was added to the mixture and the resulting solution was washed with water. The organic layer was separated and dried over sodium sulfate. The mixture was filtered and the filtrate was evaporated under reduced pressure. The crude product was purified by silica gel flash column chromatography (eluent: dichloromethane/methanol, mixture of increasing polarity). The crude amido-ester, 45 (0.195 g) and the crude amide 46 (0.270 g) were isolated and used for the next step without further purification.

To a solution of 45 (0.195 g, 0.29 mmol) in a mixture tetrahydrofuran/methanol (1:1) (5 mL) was added a 2N aqueous solution of sodium hydroxide (1 mL). The resulting mixture was stirred at room temperature for 1 h. The mixture was then concentrated in vacuo. The residue was diluted in water and the solution was acidified to pH 5 with glacial acetic acid. The solution was then concentrated to dryness. To a solution of 46 (0.270 g, 0.54 mmol) in a mixture tetrahydrofuran/methanol (1:1) (8 mL) was added a 2N aqueous solution of sodium hydroxide (1.5 mL). The resulting mixture was stirred at room temperature for 1 h. The mixture was then concentrated in vacuo. The residue was diluted in water and the solution was acidified to pH 5 with glacial acetic acid. The solution was then concentrated to dryness. The combined crude product 47 obtained by basic hydrolysis of 45 and 46 was purified by preparative HPLC [Genesis C18 column (Jones Chromatography), eluent: 20-50% acetonitrile in water (+0.1% TFA), 47 (56 mg, 30%); [M+H]⁺ 489.

### Example 85

### Preparation of 4,5α-Epoxy-3-hydroxy-14β-[(4-carboxy)propionylamino]-6-keto-17-(cyclopropylmethyl)morphinan (51)

To a cold (0° C.) solution of the hydrochloric acid salt of 43 (0.870 g, 2.30 mmol) and diisopropylethylamine (2.42 mL, 13.7 mmol) in anhydrous dimethylformamide (10 mL) was added mono-methylglutarate (0.350 g, 2.39 mmol) followed by a solution of HATU (0.926 g, 2.41 mmol) in anhydrous dimethylformamide (3 mL). The mixture was stirred for 2 h at 0° C. and stirring was continued at room temperature for an additional 16 h. The mixture was concentrated in vacuo. Ethyl acetate was added to the mixture and the resulting solution was washed with water. The organic layer was separated and dried over sodium sulfate. The mixture was filtered and the filtrate was evaporated under reduced pressure. The crude product was purified by silica gel flash column chromatography (eluent: dichloromethane/methanol, mixture of increasing polarity). The crude amido-ester, 49 (0.825 g) and the crude amide 50 (0.288 g) were isolated and used for the next step without further purification.

To a solution of 49 (0.825 g, 1.38 mmol) in a mixture tetrahydrofuran/methanol (1:1) (15 mL) was added a 2N aqueous solution of sodium hydroxide (5 mL). The resulting mixture was stirred at room temperature for 1 h. The mixture was then concentrated in vacuo. The residue was diluted in water and the solution was acidified to pH 5 with glacial acetic acid. The solution was then concentrated to dryness. The crude product 51 was purified by preparative HPLC [Genesis C18 column (Jones Chromatography), eluent: 20-50% acetonitrile in water (+0.1% TFA)], 51 (117 mg, 30%); [M+H]⁺ 455.

To a solution of 50 (0.288 g, 0.615 mmol) in a mixture tetrahydroftiran/methanol (1:1) (10 mL) was added a 2N aqueous solution of sodium hydroxide (2.5 mL). The resulting mixture was stirred at room temperature for 1 h. The mixture was then concentrated in vacuo. The residue was diluted in water and the solution was acidified to pH 5 with glacial acetic acid. The solution was then concentrated to dryness. The crude product 51 was purified by preparative HPLC [Genesis C18 column (Jones Chromatography), eluent: 20-50% acetonitrile in water (+0.1% TFA)], 51 (55 mg, 20%); [M+H]⁺ 455.

### Examples 86 and 87

### Preparation of 4,5α-Epoxy-3-hydroxy-14β-[(4-carboxy)propionylamino-6-β-hydroxy-17-(cyclopropylmethyl)morphinan (52) (Example 86) and 4,5α-Epoxy-3-hydroxy-14β-[(4-carboxy)propionylamino-6-α-hydroxy-17-(cyclopropylmethyl)morphinan (53) (Example 87)

To a solution of 51 (0.187 g, 0.32 mmol) in anhydrous methanol (30 mL) was added sodium borohydride (40 mg, 1.05 mmol). The resulting mixture was stirred at room temperature for 16 h. The solution was acidified to pH 5 with glacial acetic acid and was then concentrated to dryness. The crude product was purified by preparative HPLC [Genesis C18 column (Jones Chromatography), eluent: 5-45% acetonitrile in water (+0.1% TFA)]. Two fractions were isolated: Fraction 1: t_{R}=1.60 min, 45 mg, corresponding to the α-isomer, 53 (Example 87); [M+H]⁺ 457; Fraction 2: t_{R}=1.70 min, 8 mg, corresponding to the β-isomer, 52 (Example 86); [M+H]⁺ 457.

### Examples 88-117

### A. Preparation of 4,5α-Epoxy-3-carboxamido-14β-hydroxy-6-β-amino-17-(cyclopropylmethyl)morphinan (59)

To a solution of 54 (3 g, 5.74 mmol, 1 eq) in anhydrous dichloromethane (60 mL) was added triethylamine (1.32 mL, 9.54 mmol, 1.66 eq) followed by dropwise addition of a solution of N-triphenyltrifluoromethane sulfonimide (3.3 g, 8.4 mmol, 1.46 eq) in anhydrous dichloromethane (60 mL) at 0° C. The reaction mixture was stirred at 0° C. for 1 h and was allowed to stir at room temperature overnight. The reaction mixture was washed with aqueous 1N NaOH solution. The layers were separated and the organic phase was washed with an aqueous 1N NaOH solution. The combined organic extracts were washed with an aqueous 1N NaOH solution and dried over sodium sulfate. The mixture was filtered and the filtrate was concentrated to afford 55 used for the next step without further purification.

A solution of 55 (3.72 g, 5.74 mmol, 1 eq), palladium acetate (200 mg), diphenylphoshinopropane (357 mg), triethylamine (1.55 mL) in anhydrous dimethylformamide (53 mL) and methanol (33 mL) was purged with CO(g) for 10 minutes and the reaction mixture was stirred for 24 hours at 70° C. under a carbon monoxide atmosphere. The mixture was concentrated under vacuum. The residue was dissolved in ethyl acetate; the resulting mixture was washed with water, brine and dried over sodium sulfate. The mixture was filtered and the filtrate was concentrated. The crude product was purified by column chromatography (eluent:dichloromethane/methanol=99:1). The purified product was collected. Diethyl ether was added and the resulting mixture was stirred at room temperature. The precipitate was collected by filtration (1.68 g, 52%). For 56 mass spectral analysis: m/z=565 (M+H)⁺

A solution of 56 (0.680 g, 1.20 mmol, 1 eq) in concentrated aqueous HCl solution (25 mL) was heated at 70° C. (50 mL) for 24 h. The resulting mixture was cooled to room temperature and freeze dried affording the crude product 57 ([M+H]⁺ 551) (0.700 g) used for the next step without further purification. Note: the crude product can be purified by preparative HPLC [Genesis C18 column (Jones Chromatography), eluent: 5-50% acetonitrile in water (+0.1% TFA)].

To a suspension of the acid 57 (HCl salt) (0.600 g, 1.02 mmol, 1 eq) and triethylamine (480 μL, 3.36 mmol, 3.3 eq) in dimethylformamide (30 mL) was added ammonium chloride (0.298 g, 5.41 mmol, 5.3 eq), hydroxybenzotriazole (0.190 g, 1.3 mmol, 1.27 eq) and EDCI (0.291 g, 1.52 mmol, 1.5 eq) and the mixture was stirred for 24 hours at room temperature. The mixture was concentrated under reduced pressure and dissolved in ethyl acetate. The organic layer was separated, dried (sodium sulfate), filtered and concentrated. The crude product was dissolved in ethyl acetate and the mixture was filtered through a short silica gel column. The filtrate was concentrated affording the desired compound 58 (0.555 g, 98%); Mass spectral analysis: m/z=550 (M+H)⁺

To a solution of 58 (0.30 g, 0.546 mmol, 1 eq) in methanol (10 mL) was added Pd(OH)₂ [20% by weight dry Pd on wet carbon (water <50%), 0.10 g]. The reaction vessel was set up on a Parr shaker at 60 psi of hydrogen and shaken for 16 hours. The reaction mixture was filtered through Celite, and the celite was washed with methanol and methylene chloride. The solution was concentrated by rotary evaporation to give 240 mg of a crude solid, which was shown by LC/MS to contain the desired product mixed with mono-benzylated material and starting material. The crude solid was hydrogenated under the same conditions as described above. The resulting crude product was purified by silica gel flash column chromatography (eluent: dichloromethane/methanol/ammonium hydroxide) affording the desired amine 59 (85 mg, 42%); Mass spectral analysis: m/z=370 (M+H)⁺

### B. Preparation of Examples 88-117 from Amine 59

Polymeric tetrafluorophenol sulfonates 60 and esters 61 were prepared according to literature procedures (Salvino, J. M., et al., J. Comb. Chem. 2000, 2, 691-697).

Preparation of amides and sulfonamides (examples 88-117): Polymeric tetrafluorophenol sulfonates 60 and esters 61 (30-50 mg, ca. 26-44 μmol) were distributed into 2 mL deep well plates. The amine 59 (88.7 mg, 0.24 mmol) was dissolved in anhydrous DMF (9 mL) and 300 μl (8 μmol) of the amine stock solution were added to each resin, followed by DMF (200 μl). The resin mixtures were reacted at room temperature for 16 h. The crude reactions were diluted with methanol (500 μl), the resins were then filtered and the filtrates were collected and evaporated to dryness. Crude compounds were purified by preparative HPLC using the conditions described below:

Preparative LC/MS for Library Purification

System:

Gilson 215 Liquid Handler

Gilson 819 Injection Module

Gilson 306 Pumps (2) with 25 mL pump heads.

Gilson 155 dual wavelength UV/Vis detector

Gilson Unipoint version 1.2 system control software with Nebular Interface Kit

Thermo-Finnigan AQA Mass detector

Thermo-Finnigan Excaliber version 1.3 system control software

HPLC Parameters:

Control file name: Library.gct

Column: Phenomenex LUNA C18(2) 7.8×100 mm, 5 μ, P/N# 00D-4252-K0

Temperature: Ambient

Injection Volume: 0.400 mL

Flow: 8.7 mL/min.

Split ratio: 50 to 1

Mobile Phase A: 0.1% Trifluoroacetic Acid in HPLC Grade Water

Mobile Phase B: 0.1% Trifluoroacetic Acid in HPLC Grade Acetonitrile

Gradient: 90% Mobile Phase A, 10% Mobile Phase B for 0.35 minutes;

Proceed linearly to 0% Mobile Phase A, 100% Mobile Phase B over 5.50 minutes;
Hold at 100% Mobile Phase B for 0.65 minutes;

Re-Equilibrate to 90% Mobile Phase A, 10% Mobile Phase B before next injection.

Detector Parameters:

UV/Vis: λ1=220 nm, λ2=254 nm

MS: Electrospray +ve;
Cone Voltage: 30V;
Capillary Voltage: 3.10 kV;
Probe Temperature: 350° C.;
Scan Range: 150 to 700 amu;
Scan Rate: 1.7 scans per second.
   Fraction Collector Parameters:

MS Triggered with 10 mV trigger threshold.

Examples 1-117 are shown in Table 1.

TABLE 1

| Example | Name | Structure | [M + H]⁺ |
|---|---|---|---|
| 1 | 4,5α-Epoxy-3,14β-dihydroxy-6- (3,4-dimethylphenyl)-6,7- deshydro-17- (cyclopropylmethyl)morphinan | 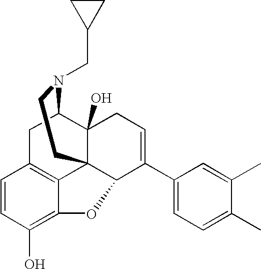 | 430.1 |
| 2 | 4,5α-Epoxy-3,14β-dihydroxy-6- (2-phenoxyphenyl)-6,7- deshydro-17- (cyclopropylmethyl)morphinan | 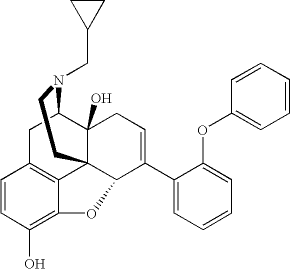 | 494.1 |
| 3 | 4,5α-Epoxy-3,14β-dihydroxy-6- (4-methylsulfonylphenyl)-6,7- deshydro-17- (cyclopropylmethyl)morphinan | 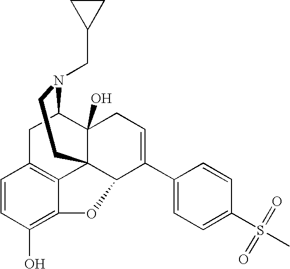 | 480.1 |
| 4 | 4,5α-Epoxy-3,14β-dihydroxy-6- (3-trifluoromethylphenyl)-6,7- deshydro-17- (cyclopropylmethyl)morphinan | 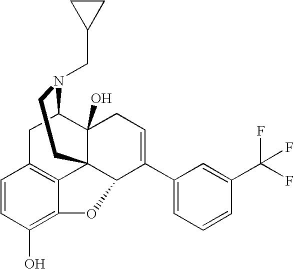 | 470.1 |
| 5 | 4,5α-Epoxy-3,14β-dihydroxy-6- (3-chlorophenyl)-6,7-deshydro- 17- (cyclopropylmethyl)morphinan | 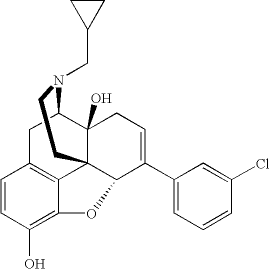 | 436.1 |
| 6 | 4,5α-Epoxy-3,14β-dihydroxy-6- (3,5-dichlorophenyl)-6,7- deshydro-17- (cyclopropylmethyl)morphinan | 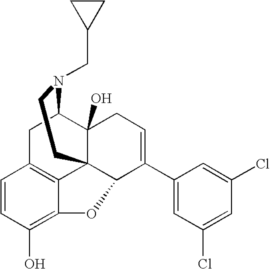 | 470.1 |
| 7 | 4,5α-Epoxy-3,14β-dihydroxy-6- (3-pyridinyl)-6,7-deshydro-17- (cyclopropylmethyl)morphinan | 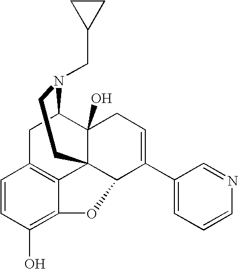 | 403.2 |
| 8 | 4,5α-Epoxy-3,14β-dihydroxy-6- 2-benzo[B]furanyl)-6,7- deshydro-17- (cyclopropylmethyl)morphinan | 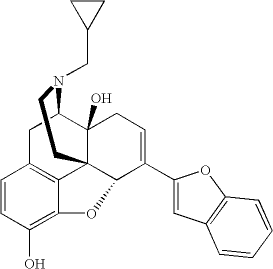 | 442.1 |
| 9 | 4,5α-Epoxy-3,14β-dihydroxy-6- (4-N,N- diethylaminocarbonylphenyl)- 6,7-deshydro-17- (cyclopropylmethyl)morphinan | 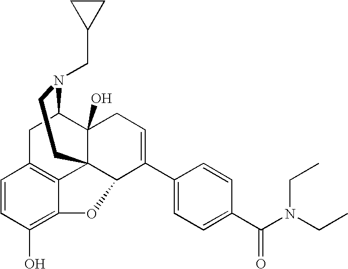 | 501.1 |
| 10 | 4,5α-Epoxy-3,14β-dihydroxy-6- (4-methoxycarbonylphenyl)-6,7- deshydro-17- (cyclopropylmethyl)morphinan | 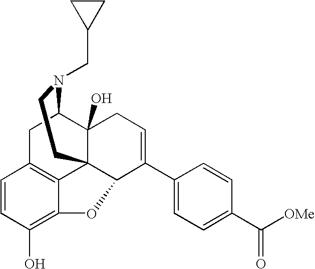 | 460.1 |
| 11 | 4,5α-Epoxy-3,14β-dihydroxy-6- phenyl-6,7-deshydro-17- (cyclopropylmethyl)morphinan | 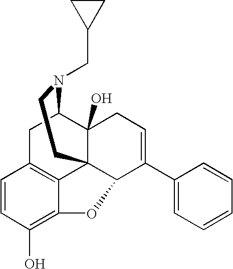 | 402.1 |
| 12 | 4,5α-Epoxy-3,14β-dihydroxy-6- (4-hydroxyphenyl)-6,7- deshydro-17- (cyclopropylmethyl)morphinan | 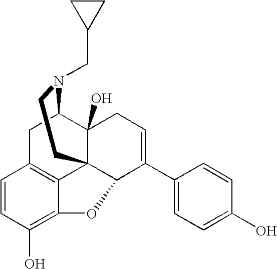 | 418.2 |
| 13 | 4,5α-Epoxy-6-carboxy-6,7- deshydro-3,14β-dihydroxy-17- (cyclopropylmethyl)morphinan | 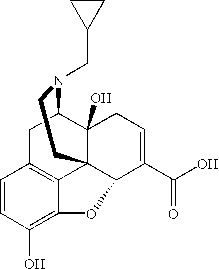 | 370.1 |
| 14 | 4,5α-Epoxy-3,14β-dihydroxy-6- (N-phenylethylaminocarbonyl)- 6,7-deshydro-17- (cyclopropylmethyl)morphinan | 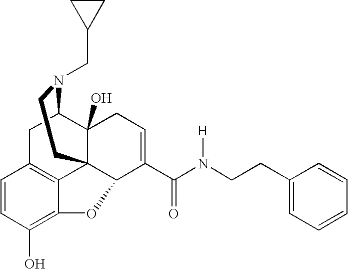 | 473.2 |
| 15 | 4,5α-Epoxy-3,14β-dihydroxy-6- (N-pentanylaminocarbonyl)-6,7- deshydro-17- (cyclopropylmethyl)morphinan | 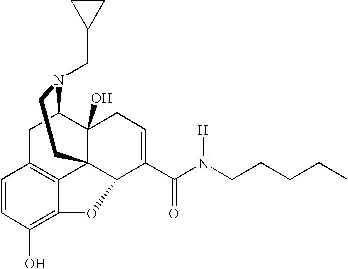 | 439.2 |
| 16 | 4,5α-Epoxy-3,14β-dihydroxy-6- [N- (ethoxyacetyl)aminocarbonyl]- 6,7-deshydro-17- (cyclopropylmethyl)morphinan | 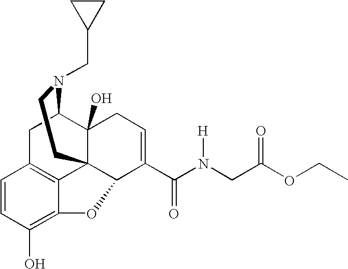 | 455.2 |
| 17 | 4,5α-Epoxy-3,14β-dihydroxy-6- [N-(tert- butoxypropionyl)aminocarbonyl]- 6,7-deshydro-17- (cyclopropylmethyl)morphinan | 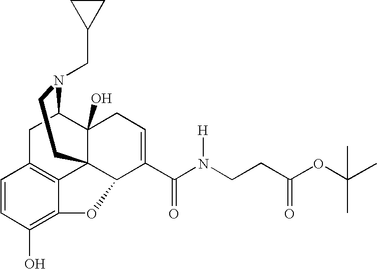 | 497.2 |
| 18 | 4,5α-Epoxy-3,14β-dihydroxy-6- [N- (methoxybutanoyl)aminocarbonyl]- 6,7-deshydro-17- (cyclopropylmethyl)morphinan | 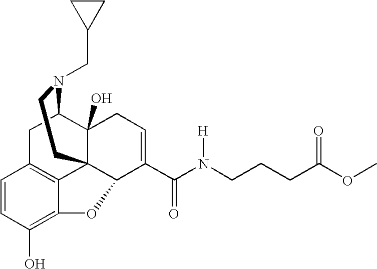 | 469.2 |
| 19 | 4,5α-Epoxy-3,14β-dihydroxy-6- [N-(4- morpholinylethyl)aminocarbonyl]- 6,7-deshydro-17- (cyclopropylmethyl)morphinan | 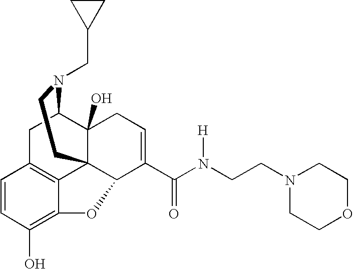 | 482.3 |
| 20 | 4,5α-Epoxy-3,14β-dihydroxy-6- {N-[1-(4- fluorophenyl)piperazinyl]amino- carbonyl]-6,7-deshydro-17- (cyclopropylmethyl)morphinan | 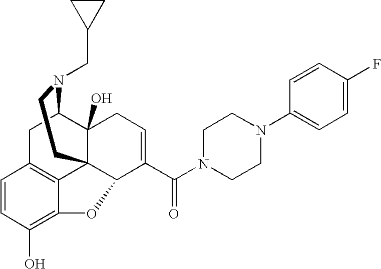 | 532.2 |
| 21 | 4,5α-Epoxy-3,14β-dihydroxy-6- {4-spiro[3-(2- pyrrolidinone)]pipendinyl}amino- carbonyl-6,7-deshydro-17- (cyclopropylmethyl)morphinan | 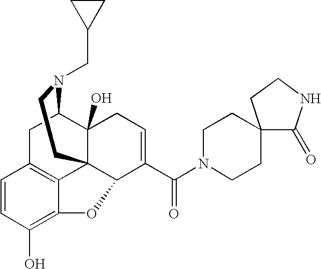 | 506.2 |
| 22 | 4,5α-Epoxy-3,14β3-dihydroxy-6- {4-spiro[3-(N-methyl-2- pyrrolidinone)]piperidinyl}amino- carbonyl-6,7-deshydro-17- (cyclopropylmethyl)morphinan | 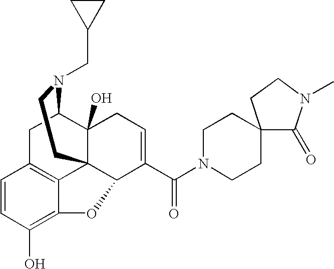 | 520.2 |
| 23 | 4,5α-Epoxy-3,14β-dihydroxy-6- {N-methyl, N-[1-(S)-isopropyl- 2-(3-(S)-hydroxy-pyrrolidin-1″- yl)]ethyl-aminocarbonyl}-6,7- deshydro-17- (cyclopropylmethyl)morphinan | 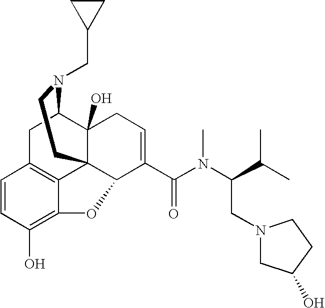 | 538.3 |
| 24 | 4,5α-Epoxy-3,14β-dihydroxy-6- {N-[4R-(3hydroxyphenyl)- 3(R),4-dimethyl-1- piperidinyl]aminocarbonyl}-6,7- deshydro-17- (cyclopropylmethyl)morphinan | 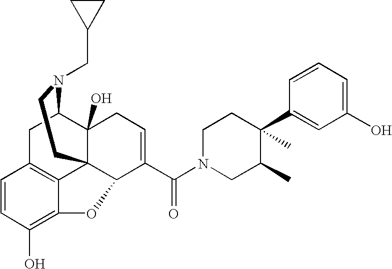 | 557.3 |
| 25 | 4,5α-Epoxy-3,14β-dihydroxy-6- [N-3-(R)- phenyl(ethoxypropionyl)amino- carbonyl]-6,7-deshydro-17- (cyclopropylmethyl)morphinan | 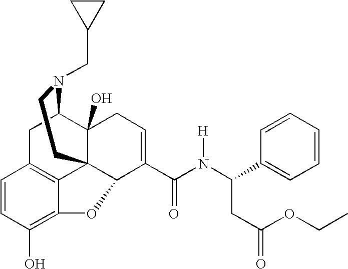 | 545.2 |
| 26 | 4,5α-Epoxy-3,14β-dihydroxy-6- [N- (methoxyacetyl)aminocarbonyl]- 6,7-deshydro-17- (cyclopropylmethyl)morphinan | 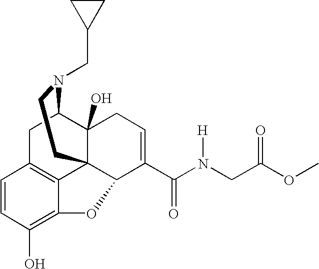 | 441.2 |
| 27 | 4,5α-Epoxy-6-methoxycarbonyl- 6,7-deshydro-3,14β-dihydroxy- 17- (cyclopropylmethyl)morphinan | 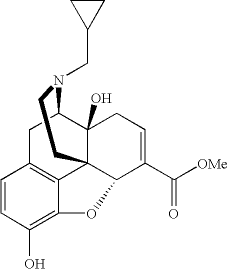 | 384.1 |
| 28 | 4,5α-Epoxy-6(α/β)- methoxycarbonyl-3,14β- dihydroxy-17- (cyclopropylmethyl)morphinan | 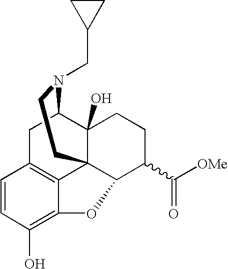 | 386.1 |
| 29 | 4,5α-Epoxy-6(α/β)-carboxy- 3,14β-dihydroxy-17- (cyclopropylmethyl)morphinan | 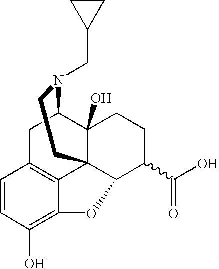 | 372.1 |
| 30 | 4,5α-Epoxy-3,14β-dihydroxy- 6,6Z(methoxycarbonylethenyl)- 17- (cyclopropylmethyl)morphinan | 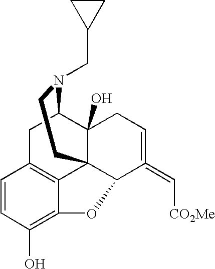 | 398.2 |
| 31 | 4,5α-Epoxy-3,14β-dihydroxy- 6,6E(methoxycarbonylethenyl)- 17- (cyclopropylmethyl)morphinan | 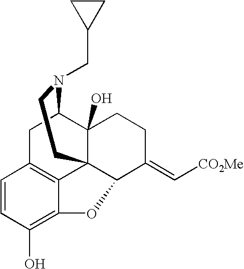 | 398.2 |
| 32 | 4,5α-Epoxy-3,14β-dihydroxy- 6,6Z(N-acryloylamino-acetic acid methyl ester)-17- (cyclopropylmethyl)morphinan | 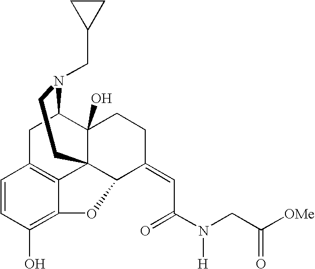 | 455.1 |
| 33 | 4,5α-Epoxy-3,14β-dihydroxy- 6,6Z(N-acryloylamino-acetic acid)-17- (cyclopropylmethyl)morphinan | 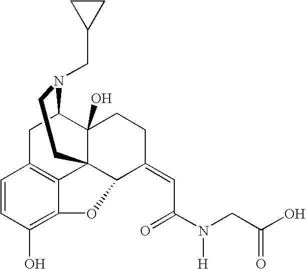 | 441.0 |
| 34a | 4,5α-Epoxy-3,14β-dihydroxy- 6,6Z(N-acryloyl-piperidine- 2(R/S)-carboxylic acid ethyl ester)-17- (cyclopropylmethyl)morphinan | 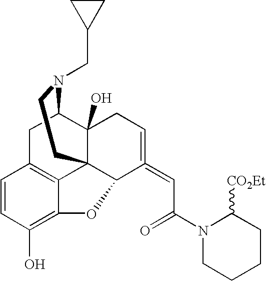 | 523.2 |
| 35 | 4,5α-Epoxy-3,14β-dihydroxy- 6,6Z(N-acryloyl-piperidine- 2(R/S)-carboxylic acid methyl ester)-17- (cyclopropylmethyl)morphinan | 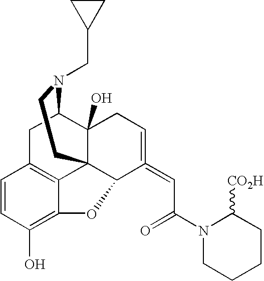 | 495.1 |
| 36 | 4,5α-Epoxy-3,14β-dihydroxy- 6,6Z(N-acryloyl-piperidine- 3(R/S)-carboxylic acid ethyl ester)-17- (cyclopropylmethyl)morphinan | 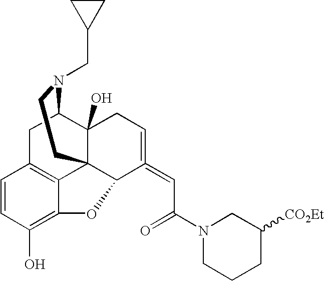 | 523.1 |
| 37 | 4,5α-Epoxy-3,14β-dihydroxy- 6,6Z(N-acryloyl-piperidine- 3(R/S)-carboxylic acid)-17- (cyclopropylmethyl)morphinan | 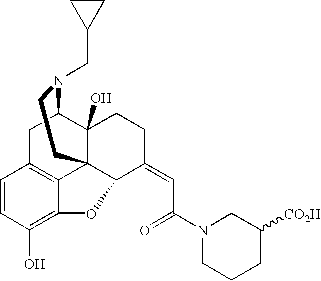 | 495.1 |
| 38 | 4,5α-Epoxy-3,14β-dihydroxy- 6,6E(carboxyethenyl)-17- (cyclopropylmethyl)morphinan | 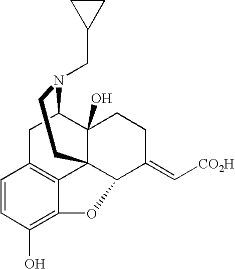 | 384.1 |
| 39 | 4,5α-Epoxy-3,14β-dihydroxy- 6,6E(N-acryloylamino-acetic acid methyl ester)-17- (cyclopropylmethyl)morphinan | 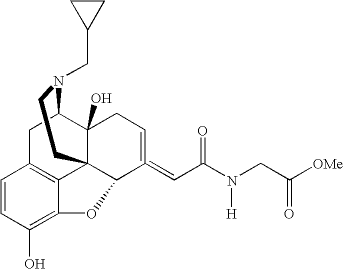 | 455.1 |
| 40 | 4,5α-Epoxy-3,14β-dihydroxy- 6,6E(N-acryloylamino-acetic acid)-17- (cyclopropylmethyl)morphinan | 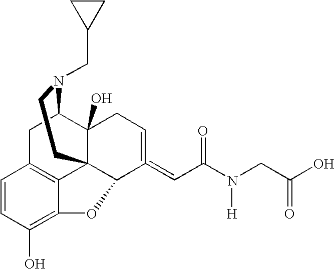 | 441.0 |
| 41 | 4,5α-Epoxy-3,14α-dihydroxy- 6,6E(N-acryloylamino-butyric acid methyl ester)-17- (cyclopropylmethyl)morphinan | 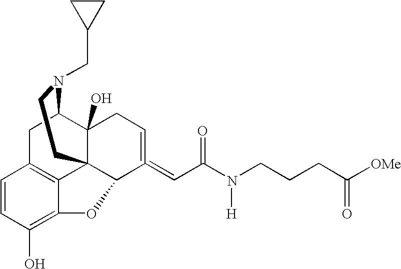 | 483.1 |
| 42 | 4,5α-Epoxy-3,14β-dihydroxy- 6,6E(N-acryloylamino-butyric acid)-17- (cyclopropylmethyl)morphinan | 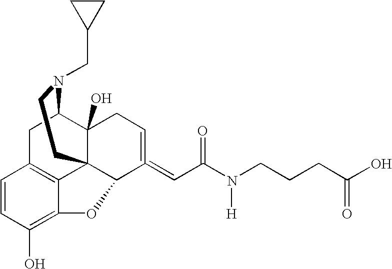 | 469.1 |
| 43 | 4,5α-Epoxy-3,14β3-dihydroxy- 6,6E(N-acryloyl-piperidine- 2(R/S)-carboxylic acid ethyl ester)-17- (cyclopropylmethyl)morphinan | 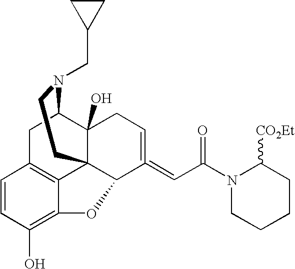 | 523.2 |
| 44 | 4,5α-Epoxy-3,14β-dihydroxy- 6,6E(N-acryloyl-piperidine- 2(R/S)-carboxylic acid)-17- (cyclopropylmethyl)morphinan | 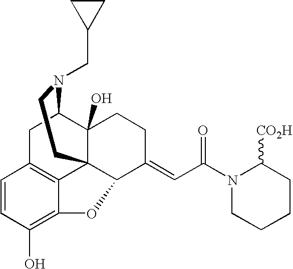 | 495.1 |
| 45 | 4,5α-Epoxy-3,14β-dihydroxy- 6,6E(N-acryloyl-piperidine- 3(R/S)-carboxylic acid ethyl ester)-17- (cyclopropylmethyl)morphinan | 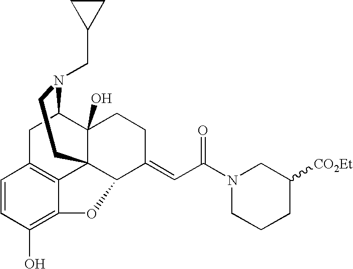 | 523.2 |
| 46 | 4,5α-Epoxy-3,14β-dihydroxy- 6,6E(N-acryloyl-piperidine- 3(R/S)-carboxylic acid)-17- (cyclopropylmethyl)morphinan | 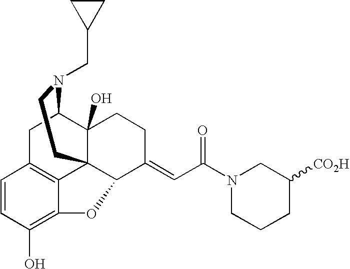 | 495.1 |
| 47 | 4,5α-Epoxy-3,14β-dihydroxy- 6,6E(N-acryloyl-piperidine-4- carboxylic acid ethyl ester)-17- (cyclopropylmethyl)morphinan | 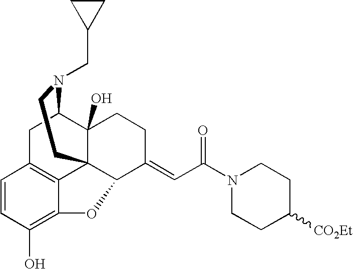 | 523.2 |
| 48 | 4,5α-Epoxy-3,14β-dihydroxy- 6,6E(N-acryloyl-piperidine-4- carboxylic acid)-17- (cyclopropylmethyl)morphinan | 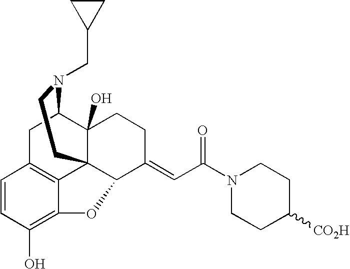 | 495.1 |
| 49 | 4,5α-Epoxy-3,14β-dihydroxy- 6,6E[N-(3(R/S)-hydroxymethyl- piperidin-1 yl)-propenone]-17- (cyclopropylmethyl)morphinan | 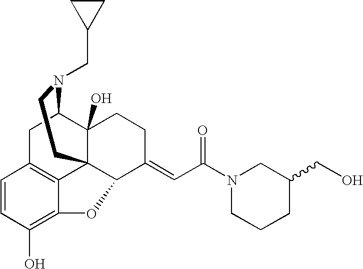 | 481.1 |
| 50 | 4,5α-Epoxy-3,14β-dihydroxy- 6,6E[N-(4-hydroxymethyl- piperidin-1 yl)-propenone]-17- (cyclopropylmethyl)morphinan | 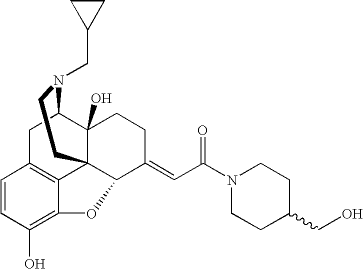 | 481.2 |
| 51 | 4,5α-Epoxy-3,14β-dihydroxy- 6,6E(N-acryloyl-piperidine- 3(R/S)-carboxamide)-17- (cyclopropylmethyl)morphinan | 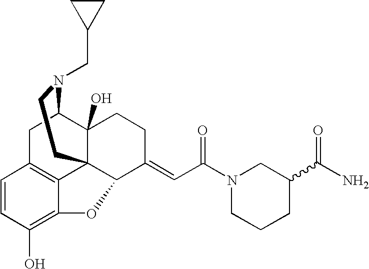 | 494.1 |
| 52 | 4,5α-Epoxy-3,14β-dihydroxy- 6,6E(N-acryloyl-piperidine-4- carboxamide)-17- (cyclopropylmethyl)morphinan | 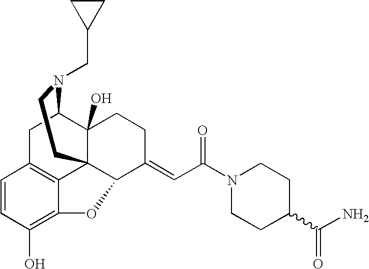 | 494.2 |
| 53 | 4,5α-Epoxy-3,14β-dihydroxy- 6,6E(2- methoxyethylaminopropenone)- 17- (cyclopropylmethyl)morphinan | 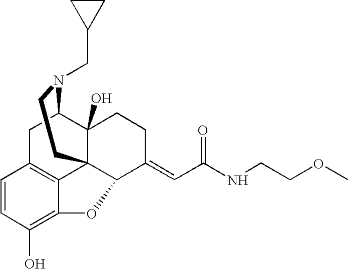 | 441.0 |
| 54 | 4,5α-Epoxy-3,14β-dihydroxy- 6(R/S)-(acetylaminoacetic acid methyl ester)-17- (cyclopropylmethyl)morphinan | 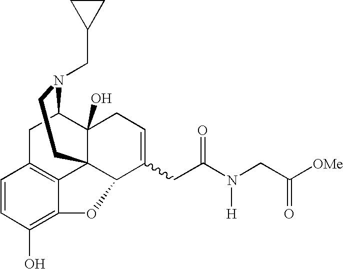 | 457.0 |
| 55 | 4,5α-Epoxy-3,14β-dihydroxy- 6,6Z(hydroxyethylidene)-17- (cyclopropylmethyl)morphinan | 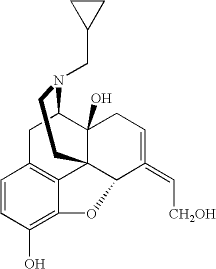 | 370.0 |
| 56 | 4,5α-Epoxy-3,14β-dihydroxy- 6,6E(hydroxyethylidene)-17- (cyclopropylmethyl)morphinan | 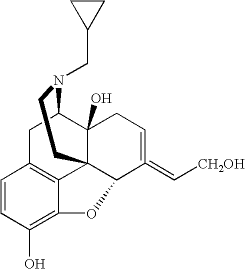 | 370.0 |
| 57 | 4,5α-Epoxy-3,14β-dihydroxy-6- ethylene-6,7-deshydro-17- (cyclopropylmethyl)morphinan | 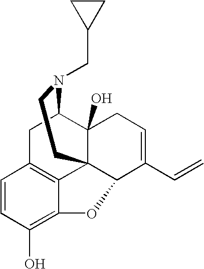 | 352.2 |
| 58 | 4,5α-Epoxy-3-hydroxy-14β- acetylamino-6-β-benzylamino- 17- (cyclopropylmethyl)morphinan | 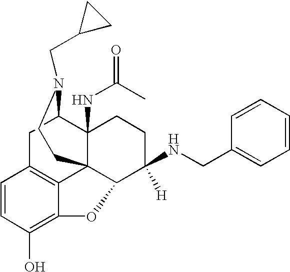 | 474.2 |
| 59 | 4,5α-Epoxy-3-hydroxy-14β- acetylamino-6-β-(4- chlorobenzylamino)-17- (cyclopropylmethyl)morphinan | 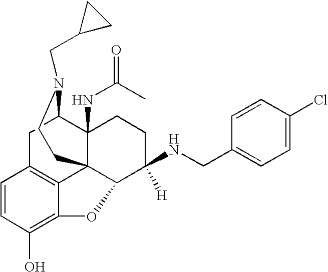 | 508.3 |
| 60 | 4,5α-Epoxy-3-hydroxy-14β- acetylamino-6-β-(c-pyridinyl-3- yl-methylamino)-17- (cyclopropylmethyl)morphinan | 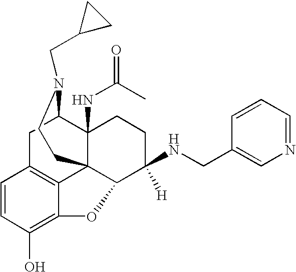 | 475.2 |
| 61 | 4,5α-Epoxy-3-hydroxy-14β- acetylamino-6-β-[(4- (methyloxycarbonyl)benzyl- amino]-17- (cyclopropylmethyl)morphinan | 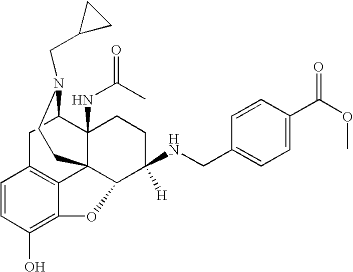 | 532.3 |
| 62 | 4,5α-Epoxy-3-hydroxy-14β- acetylamino-6-β-[3,4- dichlorobenzylamino]-17- (cyclopropylmethyl)morphinan | 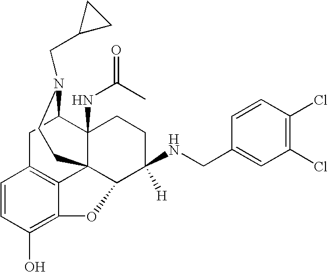 | 542.2 |
| 63 | 4,5α-Epoxy-3-hydroxy-14β- acetylamino-6-β-[(2-tert- butyloxycarbonyl,2- (R)propyl)methylamino]-17- (cyclopropylmethyl)morphinan | 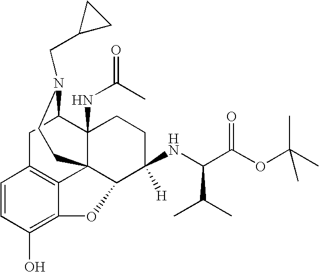 | 540.3 |
| 64 | 4,5α-Epoxy-3-hydroxy-14β- acetylamino-6-β-(phenylamino)- 17- (cyclopropylmethyl)morphinan | 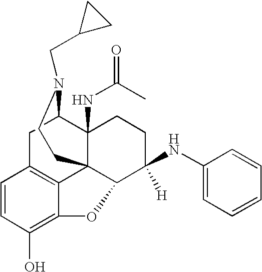 | 460.4 |
| 65 | 4,5α-Epoxy-3-hydroxy-14β- acetylamino-6-β-(butylamino)- 17- (cyclopropylmethyl)morphinan | 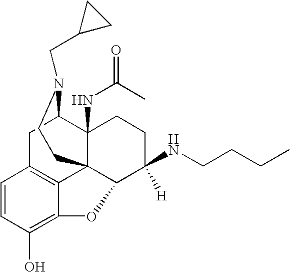 | 440.3 |
| 66 | 4,5α-Epoxy-3-hydroxy-14β- acetylamino-6-β-(tert- butyloxycarbonylmethylamino)- 17- (cyclopropylmethyl)morphinan | 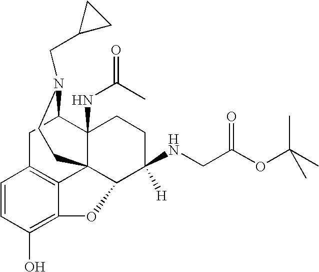 | 498.6 |
| 67 | 4,5α-Epoxy-3-hydroxy-14β- acetylamino-6-β-[2-(3,4- dimethoxyphenyl)ethylamino]- 17- (cyclopropylmethyl)morphinan | 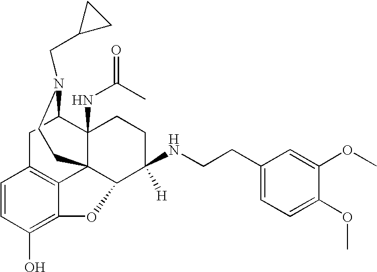 | 548.3 |
| 68 | 4,5α-Epoxy-3-hydroxy-14β- acetylamino-6-β-(2- morpholinyl-4-yl-ethylamino]- 17- (cyclopropylmethyl)morphinan | 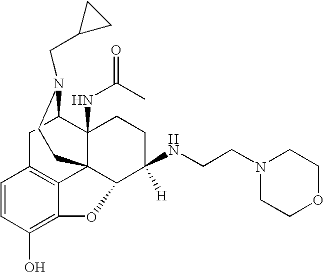 | 497.3 |
| 69 | 4,5α-Epoxy-3-hydroxy-14β- acetylamino-6β-piperidino-14β- (cyclopropylmethyl)morphinan | 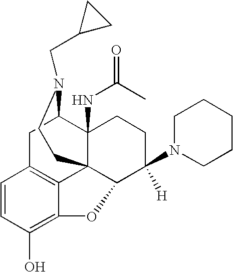 | 452.2 |
| 70 | 4,5α-Epoxy-3-hydroxy-14β- acetylamino-6 β-morpholino-17- (cyclopropylmethyl)morphinan | 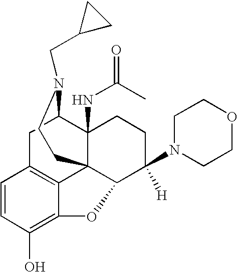 | 454.2 |
| 71 | 4,5α-Epoxy-3-hydroxy-14β- acetylamino-6 β-N-benzyl-N- methylamino-17- (cyclopropylmethyl)morphinan | 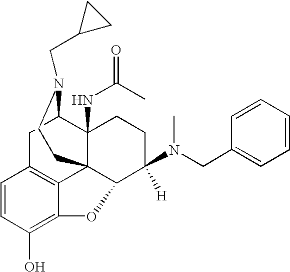 | 488.3 |
| 72 | 4,5α-Epoxy-3-hydroxy-14β- acetylamino-6 β-[(4- ethyloxycarbonyl)piperidino]- 17- (cyclopropylmethyl)morphinan | 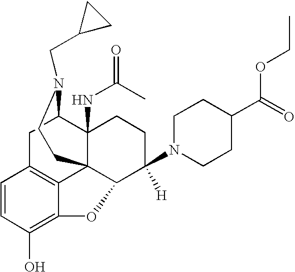 | 524.4 |
| 73 | 4,5α-Epoxy-3-hydroxy-14β- acetylamino-6-α-benzylamino- 17- (cyclopropylmethyl)morphinan | 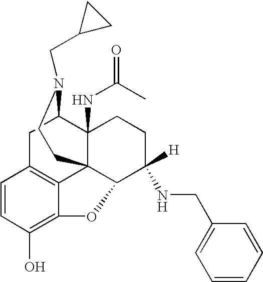 | 474.2 |
| 74 | 4,5α-Epoxy-3-hydroxy-14β- acetylamino-6-α-(4- chlorobenzylamino)-17- (cyclopropylmethyl)morphinan | 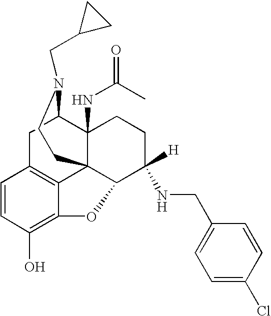 | 508.3 |
| 75 | 4,5α-Epoxy-3-hydroxy-14β- acetylamino-6-α-[3,4- dichlorobenzylamino]-17- (cyclopropylmethyl)morphinan | 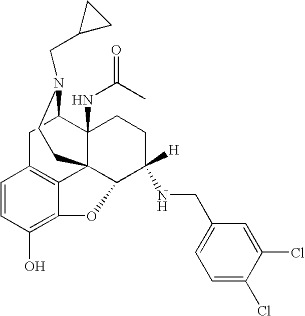 | 542.2 |
| 76 | 4,5α-Epoxy-3-hydroxy-14β- acetylamino-6-α-[(2-tert- butyloxycarbonyl,2- (R)propyl)methylamino]-17- (cyclopropylmethyl)morphinan | 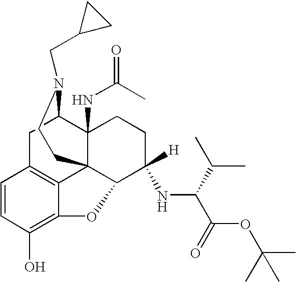 | 540.3 |
| 77 | 4,5α-Epoxy-3-hydroxy-14β- acetylamino-6-α-(phenylamino)- 17- (cyclopropylmethyl)morphinan | 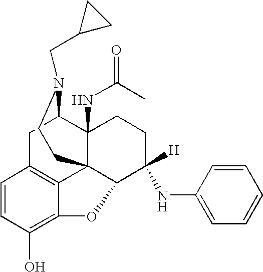 | 460.2 |
| 78 | 4,5α-Epoxy-3-hydroxy-14β- acetylamino-6-α-(butylamino)- 17- (cyclopropylmethyl)morphinan | 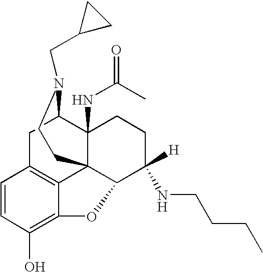 | 440.3 |
| 79 | 4,5α-Epoxy-3-hydroxy-14β- acetylamino-6-α-(tert- butyloxycarbonylmethylamino)- 17- (cyclopropylmethyl)morphinan | 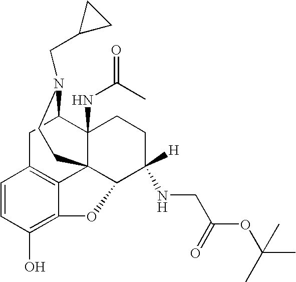 | 498.3 |
| 80 | 4,5α-Epoxy-3-hydroxy-14β- acetylamino-6-α-[2-(3,4- dimethoxyphenyl)ethylamino]- 17- (cyclopropylmethyl)morphinan | 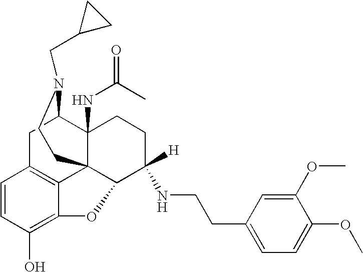 | 548.3 |
| 81 | 4,5α-Epoxy-3-hydroxy-14β- acetylamino-6 β- carboxymethylamino-17- (cyclopropylmethyl)morphinan | 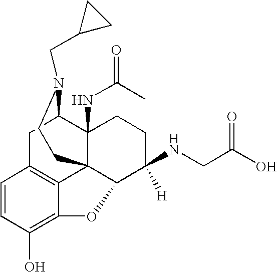 | 442.2 |
| 82 | 4,5α-Epoxy-3-hydroxy-14β- acetylamino-6 β-(4- carboxypiperidino)-17- (cyclopropylmethyl)morphinan | 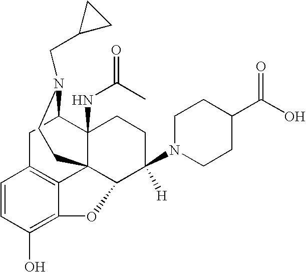 | 496.2 |
| 83 | 4,5α-Epoxy-3-hydroxy-14β- acetylamino-6 β-(4- carboxybenzylamino)-17- (cyclopropylmethyl)morphinan | 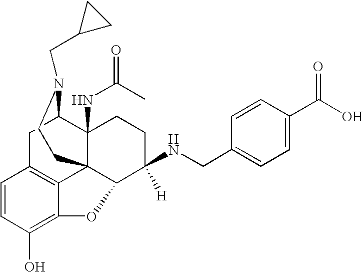 | 518.3 |
| 84 | 4,5α-Epoxy-3-hydroxy-14β-[(4- carboxy)phenylcarbonylamino]- 6-keto-17- (cyclopropylmethyl)morphinan | 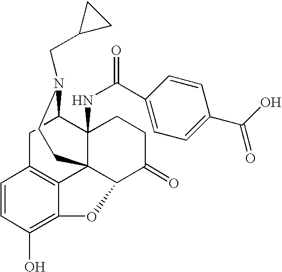 | 489.5 |
| 85 | 4,5α-Epoxy-3-hydroxy-14β-[(4- carboxy)propionylamino]- 6-keto-17- (cyclopropylmethyl)morphinan | 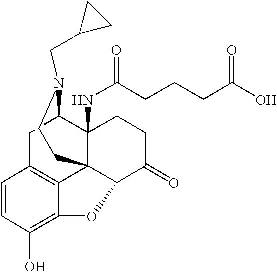 | 455.3 |
| 86 | 4,5α-Epoxy-3-hydroxy-14β-[(4- carboxy)propionylamino]- 6-β-hydroxy-17- (cyclopropylmethyl)morphinan | 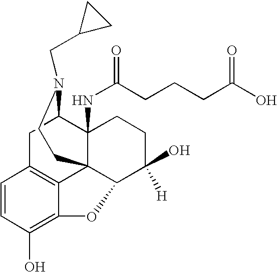 | 457.4 |
| 87 | 4,5α-Epoxy-3-hydroxy-14β-[(4- carboxy)propionylamino]- 6-α-hydroxy-17- (cyclopropylmethyl)morphinan | 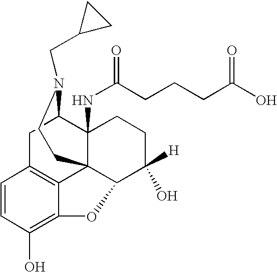 | 457.4 |
| 88 | 4,5α-Epoxy-3-carboxamido- 14β-hydroxy-6-β- propanesulfonylamino-17- (cyclopropylmethyl)morphinan | 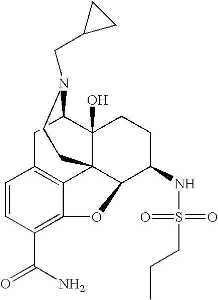 | 476.2 |
| 89 | 4,5α-Epoxy-3-carboxamido- 14β-hydroxy-6-β- butanesulfonylamino-17- (cyclopropylmethyl)morphinan | 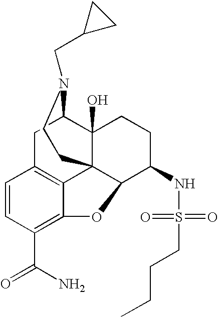 | 490.2 |
| 90 | 4,5α-Epoxy-3-carboxamido- 14β3-hydroxy-6-β- benzenesulfonylamino-17- (cyclopropylmethyl)morphinan | 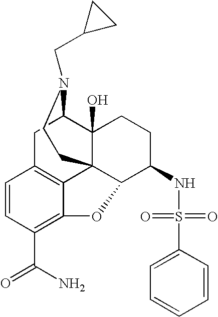 | 510.2 |
| 91 | 4,5α-Epoxy-3-carboxamido- 14β-hydroxy-6-β- ethanesulfonylamino-17- (cyclopropylmethyl)morphinan | 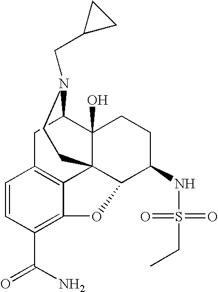 | 462.2 |
| 92 | 4,5α-Epoxy-3-carboxamido- 14β-hydroxy-6-β-[(4-fluoro- phenyl)acetyl]amino-17- (cyclopropylmethyl)morphinan | 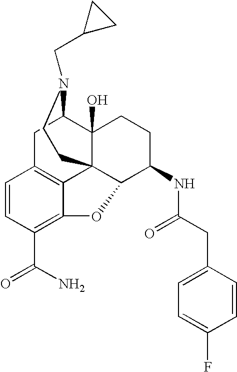 | 506.2 |
| 93 | 4,5α-Epoxy-3-carboxamido- 14β-hydroxy-6-β-[(4-methoxy- phenyl)acetyl]amino-17- (cyclopropylmethyl)morphinan | 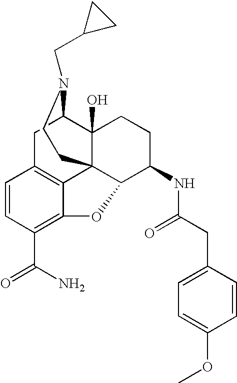 | 518.3 |
| 94 | 4,5α-Epoxy-3-carboxamido- 14β-hydroxy-6-β-(3-methyl-2- phenylpropionyl)amino-17- (cyclopropylmethyl)morphinan | 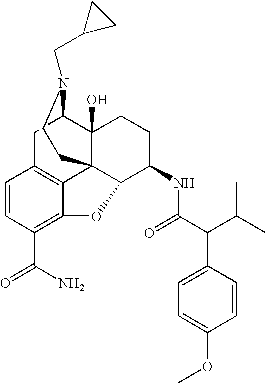 | 530.3 |
| 95 | 4,5α-Epoxy-3-carboxamido- 14β-hydroxy-6-β-(2,2- dimethylpropionyl)amino-17- (cyclopropylmethyl)morphinan | 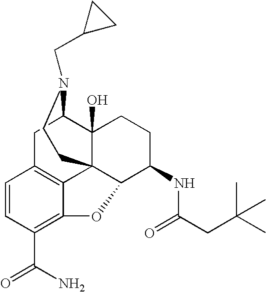 | 468.3 |
| 96 | 4,5α-Epoxy-3-carboxamido- 14β-hydroxy-6-β-(2- thienylcarbonyl)amino-17- (cyclopropylmethyl)morphinan | 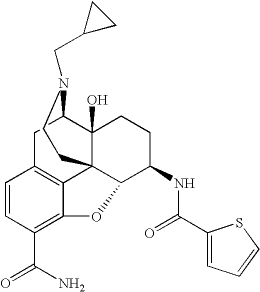 | 480.2 |
| 97 | 4,5α-Epoxy-3-carboxamido- 14β-hydroxy-6-β-(4-methylthio- benzoyl)amino-17- (cyclopropylmethyl)morphinan | 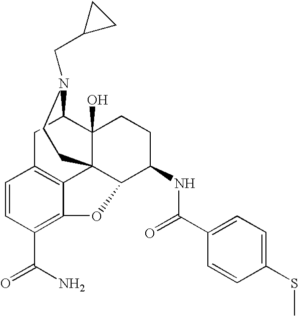 | 520.2 |
| 98 | 4,5α-Epoxy-3-carboxamido- 14β-hydroxy-6-β-(4-chloro- benzoyl)amino-17- (cyclopropylmethyl)morphinan | 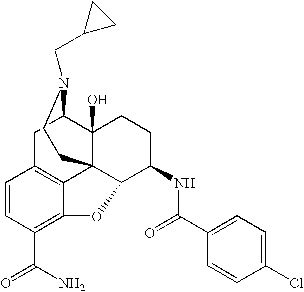 | 508.2 |
| 99 | 4,5α-Epoxy-3-carboxamido- 14β-hydroxy-6-β-(4- acetamidobutanoyl)amino-17- (cyclopropylmethyl)morphinan | 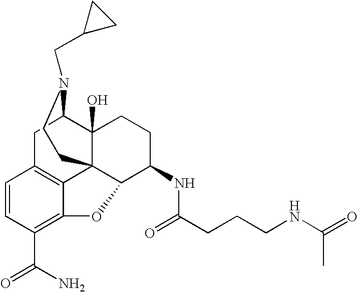 | 497.3 |
| 100 | 4,5α-Epoxy-3-carboxamido- 14β-hydroxy-6-β-(2- furoyl)amino-17- (cyclopropylmethyl)morphinan | 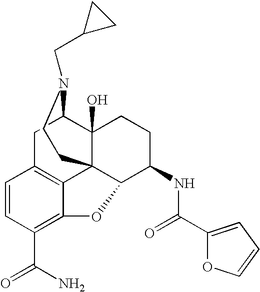 | 464.2 |
| 101 | 4,5α-Epoxy-3-carboxamido- 14β-hydroxy-6-β-(α- methylcinnamoyl)amino-17- (cyclopropylmethyl)morphinan | 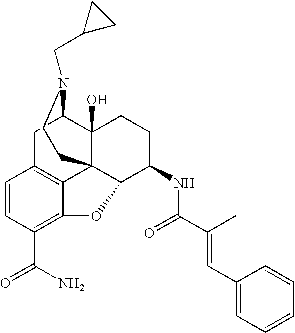 | 514.3 |
| 102 | 4,5α-Epoxy-3-carboxamido- 14β3-hydroxy-6-β-(6- chloronicotmoyl)amino-17- (cyclopropylmethyl)morphinan | 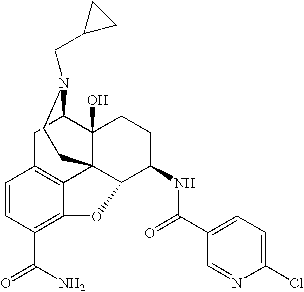 | 509.2 |
| 103 | 4,5α-Epoxy-3-carboxamido- 14β-hydroxy-6-β-(6- quinoxalinecarbonyl)amino-17- (cyclopropylmethyl)morphinan | 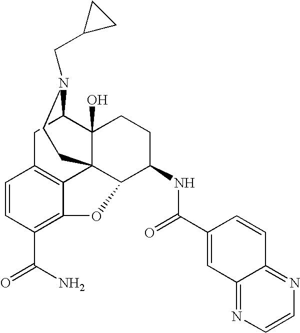 | 526.2 |
| 104 | 4,5α-Epoxy-3-carboxamido- 14β-hydroxy-6-β-(3-chloro-4- methylbenzoyl)amino-17- (cyclopropylmethyl)morphinan | 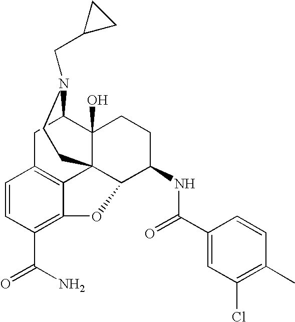 | 522.2 |
| 105 | 4,5α-Epoxy-3-carboxamido- 14β-hydroxy-6-β-[(S)-α- methoxy-phenylacetyl]amino- 17- (cyclopropylmethyl)morphinan | 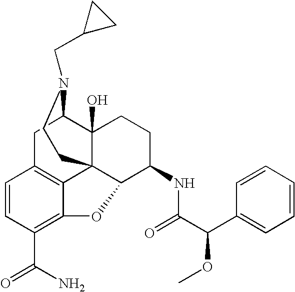 | 518.3 |
| 106 | 4,5α-Epoxy-3-carboxamido- 14β3-hydroxy-6-β-(2,4-dimethyl- 5-thiazolylcarbonyl)amino-17- (cyclopropylmethyl)morphinan | 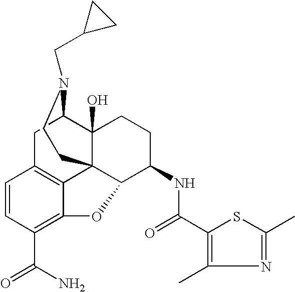 | 509.2 |
| 107 | 4,5α-Epoxy-3-carboxamido- 14β-hydroxy-6-β-(quinoline-4- carbonyl)amino-17- (cyclopropylmethyl)morphinan | 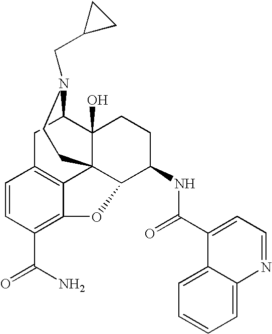 | 525.2 |
| 108 | 4,5α-Epoxy-3-carboxamido- 14β-hydroxy-6-β-(1,2,3- thiadiazole-4-carbonyl)amino- 17- (cyclopropylmethyl)morphinan | 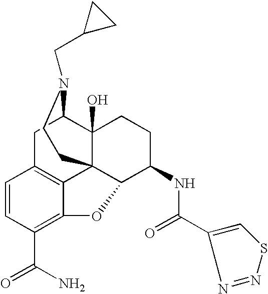 | 482.2 |
| 109 | 4,5α-Epoxy-3-carboxamido- 14β-hydroxy-6-β-benzoylamino- 17- (cyclopropylmethyl)morphinan | 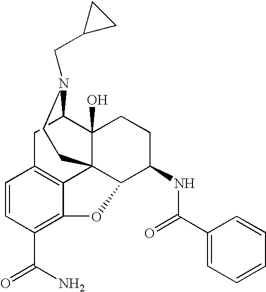 | 474.2 |
| 110 | 4,5α-Epoxy-3-carboxamido- 14β-hydroxy-6-β-acetamido-17- (cyclopropylmethyl)morphinan | 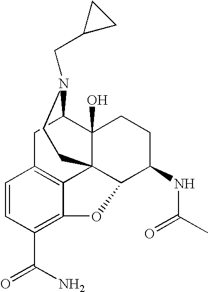 | 412.2 |
| 111 | 4,5α-Epoxy-3-carboxamido- 14β3-hydroxy-6-β-(4- methoxycarbonylbenzoy)lamino- 17- (cyclopropylmethyl)morphinan | 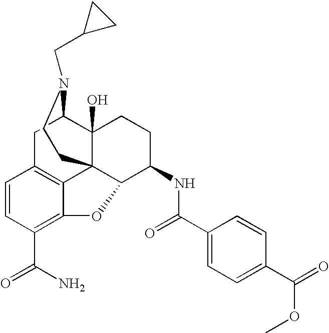 | 532.2 |
| 112 | 4,5α-Epoxy-3-carboxamido- 14β3-hydroxy-6-β-(2,3- dihydrobenzo[b]furan-5- carbonyl)amino-17- (cyclopropylmethyl)morphinan | 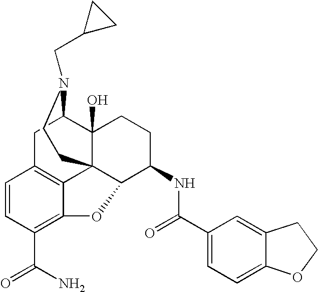 | 516.2 |
| 113 | 4,5α-Epoxy-3-carboxamido- 14β-hydroxy-6-β- (methylthioacetyl)amino-17- (cyclopropylmethyl)morphinan | 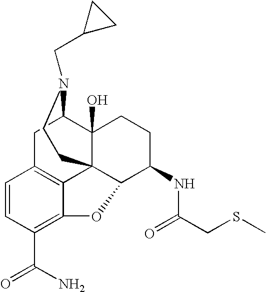 | 458.2 |
| 114 | 4,5α-Epoxy-3-carboxamido- 14β-hydroxy-6-β-(1H-pyrrole-2- carbonyl)amino-17- (cyclopropylmethyl)morphinan | 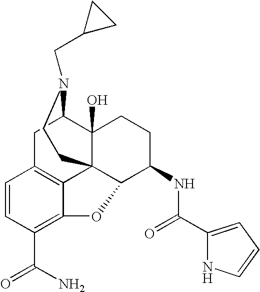 | 463.2 |
| 115 | 4,5α-Epoxy-3-carboxamido- 14β-hydroxy-6-β-(1H-1,2,3- benzotriaxole-5-carbonyl)amino- 17- (cyclopropylmethyl)morphinan | 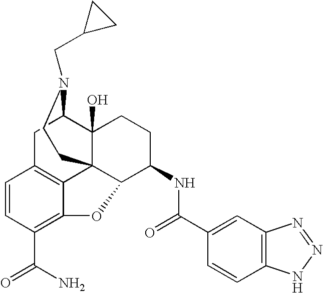 | 515.2 |
| 116 | 4,5α-Epoxy-3-carboxamido- 14β-hydroxy-6-β-(1- methylcyclopropane-1- carbonyl)amino-17- (cyclopropylmethyl)morphinan | 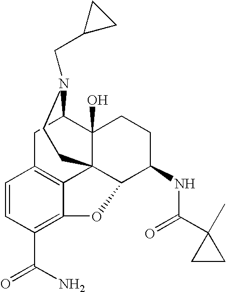 | 452.2 |
| 117 | 4,5α-Epoxy-3-carboxamido- 14β-hydroxy-6-β-(indole-3- carbonyl)amino-17- (cyclopropylmethyl)morphinan | 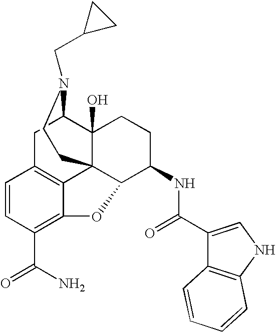 | 513.2 |

Biological Assays

The potencies of the compounds were determined by testing the ability of a range of concentrations of each compound to inhibit the binding of the non-selective opioid antagonist, [³H]diprenorphine, to the cloned human μ, κ, and δ opioid receptors, expressed in separate cell lines. IC₅₀ values were obtained by nonlinear analysis of the data using GraphPad Prism version 3.00 for Windows (GraphPad Software, San Diego). Kᵢ values were obtained by Cheng-Prusoff corrections of IC₅₀ values.

Receptor Binding (in vitro Assay)

The receptor binding method (DeHaven and DeHaven-Hudkins, “Characterization of Opioid Receptors”, Current Protocols in Pharmacology (Eds. Enna S J and Williams M) 1.4.1-1.4.12, John Wiley & Sons, Inc., New York (1998)) was a modification of the method of Raynor, et al., Mol. Pharmacol. 45:330-334 (1994). After dilution in buffer A and homogenization as before, membrane proteins (10-80 μg) in 250 μL were added to mixtures containing test compound and [³H]diprenorphine (0.5 to 1.0 nM, 40,000 to 50,000 dpm) in 250 μL of buffer A in 96-well deep-well polystyrene titer plates (Beckman). After incubation at room temperature for one hour, the samples were filtered through GF/B filters that had been pre-soaked in a solution of 0.5% (w/v) polyethylenimine and 0.1% (w/v) bovine serum albumin in water. The filters were rinsed 4 times with 1 mL of cold 50 mM Tris HCl, pH 7.8 and radioactivity remaining on the filters determined by scintillation spectroscopy. Nonspecific binding was determined by the minimum values of the titration curves and was confirmed by separate assay wells containing 10 μM naloxone. Kᵢ values were determined by Cheng-Prusoff corrections of IC₅₀ values derived from nonlinear regression fits of 12 point titration curves using GraphPad Prism® version 3.00 for Windows (GraphPad Software, San Diego, Calif.).

To determine the equilibrium dissociation constant for the inhibitors (Kᵢ), radioligand bound (cpm) in the presence of various concentrations of test compounds was measured. The concentration to give half-maximal inhibition (EC₅₀) of radioligand binding was determined from a best nonlinear regression fit to the following equation,

$$Y = Bottom + \frac{( Top - Bottom )}{1 + {10}^{X - Log EC50}}$$

where Y is the amount of radioligand bound at each concentration of test compound, Bottom is the calculated amount of radioligand bound in the presence of an infinite concentration of test compound, Top is the calculated amount of radioligand bound in the absence of test compound, X is the logarithm of the concentration of test compound, and LogEC₅₀ is the log of the concentration of test compound where the amount of radioligand bound is half-way between Top and Bottom. The nonlinear regression fit was performed using the program Prism® (GraphPad Software, San Diego, Calif.). The Kᵢ values were then determined from the EC₅₀ values by the following equation,

$$Ki = \frac{{EC}_{50}}{1 + \frac{[ ligand ]}{{K}_{d}}}$$

where [ligand] is the concentration of radioligand and K_{d} is the equilibrium dissociation constant for the radioligand.

The potencies of the antagonists were assessed by their abilities to inhibit agonist-stimulated [³⁵S]GTPγS binding to membranes containing the cloned human μ, κ, or δ opioid receptors. The agonists used were loperamide for the μ opioid receptor, U50488H for the κ opioid receptor, and BW373U86 for the δ opioid receptor.

To determine the IC₅₀ value, which was the concentration to give half-maximal inhibition of agonist-stimulated [³⁵S]GTPγS binding, the amount of [³⁵S]GTPγS bound in the presence of a fixed concentration of agonist and various concentrations of antagonist was measured. The fixed concentration of agonist was the EC₈₀ for the agonist, which was the concentration to give 80% of the relative maximum stimulation of [³⁵5]GTPγS binding. The IC₅₀ value was determined from a best nonlinear regression fit of the data to the following equation,

$$Y = Bottom + \frac{( Top - Bottom )}{1 + {10}^{X - Log IC50}}$$

where Y is the amount of [³⁵S]GTPγS bound at each concentration of antagonist, Bottom is the calculated amount of [³⁵S]GTPγS bound in the presence of an infinite concentration of antagonist, Top is the calculated amount of [³⁵S]GTPγS bound in the absence of added antagonist, X is the logarithm of the concentration of antagonist, and LogIC₅₀ is the logarithm of the concentration of antagonist where the amount of [³⁵S]GTPγS bound is halfway between Bottom and Top. The nonlinear regression fit was performed using GraphPad Prism® version 3.00 for Windows (GraphPad Software, San Diego, Calif.).
Mouse Gastrointestinal Transit (GIT) Assay (in vivo Assay)

Male Swiss-Webster mice (25-30 g) obtained from Ace Animals (Boyertown, Pa.) were used for all experiments. Mice were housed 4/cage in polycarbonate cages with food and water available ad libitum. Mice were on a 12 hours light:dark schedule with lights on at 6:30 a.m. All experiments were performed during the light cycle. Mice were fasted the night before the experiment, with water available ad libitum.

Mice were administered vehicle (10% DMSO:20% Cremophor EL:70% saline) or test compound (10 mg/kg) orally 2 or 6 hour before determination of GIT. Compounds were administered in a volume of 0.1 ml/10 g of body weight. Morphine (3 mg/kg) or vehicle (0.9% saline) was administered s.c. 35 minutes prior to determination of GIT. Ten minutes after the morphine treatment, mice were administered 0.2 ml of a charcoal meal orally. The charcoal meal consisted of a slurry of charcoal, flour, and water in the following ratio (1:2:8, w:w:v). Twenty-five minutes after receiving the charcoal meal, the mice were euthanized with CO₂ and GIT determined. GIT is expressed as the % GIT by the following formula:

$$\frac{( distance to leading edge of charcoal meal ( cm ) )}{( total length of the small intestine ( cm ) )} \times 100.$$

For each compound a % Antagonism (% A) value was determined for the 2 and 6 hour antagonist pretreatment. Using the mean % GIT for each treatment group, % A was calculated using the following formula:

$$1 - \frac{\begin{matrix}( ( mean vehicle response - \\ mean antagonist + morphine response ) )\end{matrix}}{( mean vehicle response - mean morphine response )} \times 100$$

Biological Results

Examples 1-177, listed in Table 1, were tested for their affinity toward the human cloned μ, δ and κ opioid receptors. All ligands tested bound to the human μ opioid receptor with affinity less than 100 μM. These ligands displayed various degrees of selectivity, e.g., μ vs. δ or μ vs. κ. The activity of selected ligands was also evaluated in vitro. These compounds were found to exhibit antagonist activity at μ opioid receptors (with no detectable agonist activity at concentration >10 μM). For example, ligand Example 33 (Kᵢ (μ)=3.0 nM) was found to possess potent in vitro μ receptor antagonist potency (IC₅₀=21.3 nM). Ligand Example 85 (Kᵢ (μ)=4.1 nM) was also found to possess potent in vitro μ receptor antagonist potency (IC₅₀=1.1 nM). As additional example, ligand Example 73 (Kᵢ (μ)=2.8 nM) was found to possess potent in vitro μ receptor antagonist potency (IC₅₀=4.2 nM).

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A compound of Formula Ia: 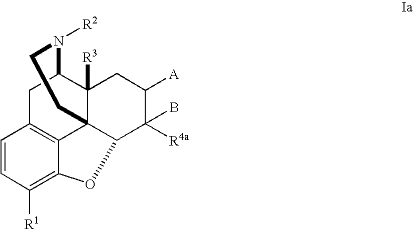
wherein:
R¹ is —OR^{5a}, —N(R^{5b})(R^{6b}), —COOR^{5c}, —CON(R^{5d})(R^{6d}), or —CH₂OR^{5e};
R² is H, alkyl, cycloalkyl, cycloalkylalkyl, aralkyl, aryl, or alkenyl;
R³ is —OR^{5f} or —N(R^{5g})—Y-Z;
each Y is independently a single bond, —[C(R^{5h})(R^{6h})]ₜ—, —C(═O)—, or —S(═O)₂—;
each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;
each W is independently —[C(R⁵ⁱ)(R⁶ⁱ)]ₜ—;
each R⁷ is independently —C(═O)—R⁸;
each R is independently —OR^{5j} or —N(R^{5k})(R^{6k});
A and B are each independently H or alkyl, or together represent a double bond between the carbon atoms to which they are attached;
R^{4a} is —[C(R^{5m})(R^{6m})]ₛ—R^{4b} or —OR⁵ⁿ, or R^{4a} and B taken together with the carbon atom to which they are attached may form: 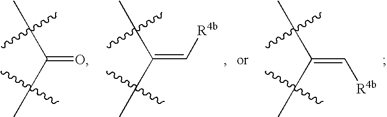
provided that when B is alkyl, then R^{4a} is —[C(R^{5m})(R^{6m})]ₛ—R^{4b};
R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;
each R^{5a}, R^{5b}, and R^{5d} is independently H, alkyl, cycloalkyl, cycloalkylalkyl, or aralkyl;
R^{5c} is aralkyl;
R^{5e} is alkyl, cycloalkyl, cycloalkylalkyl, or aralkyl;
each R^{5f}, R^{5g}, R^{5h}, R⁵ⁱ, R^{5j}, R^{5k}, R^{5m}, R⁵ⁿ, R^{5p}, R^{5q}, and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;
each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
each s is independently 0 or 1; and
each t is independently an integer from 1 to 12;
provided that:
(a) when R¹ is —OH or —O-alkyl, R² is H, cycloalkylalkyl, or alkenyl, R³ is —NR^{5g}—Y-Z, R^{5g} is H, alkyl, cycloalkylalkyl, or aralkyl, Y is —C(═O)— or a single bond, A is H, and either B is H and R^{4a} is —OR⁵ⁿ or B and R^{4a} taken together with the carbon atom to which they are attached form: 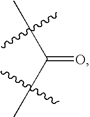
then Z is heterocycloalkyl, heteroaryl, or —WR⁷;
(b) when R¹ and R³ are both —OH, R² is alkyl, cycloalkylalkyl, aryl, aralkyl, or alkenyl, A and B are each H, and R^{4a} is —NR^{5p}—C(═O)-Z, then R^{5p} is other than H, or Z is other than alkyl;
(c) when R¹ and R³ are both —OH, R² is alkyl, cycloalkylalkyl, aryl, aralkyl, or alkenyl, A and B are each H, and R^{4a} is —NR^{5p}-Z, then at least one of R^{5p} and Z is other than haloalkyl;
(d) when R¹ is —CON(R^{5d})(R^{6d}), then:
at least one of R^{5d} and R^{6d} is cycloalkyl, or
R^{6d} is aryl, or
both R^{5d} and R^{6d} are other than H, or
R^{5d} and R^{6d}, together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
(e) when R¹ and R³ are both —OH, R² is cyclopropylmethyl, and A and B together represent a double bond between the carbon atoms to which they are attached, then R^{4a} is other than phenyl, indolyl, or pyrrolyl;
(f) when R¹ and R³ are —OH, R² is cyclopropylmethyl, alkenyl, or alkyl, and R^{4a} and B taken together with the carbon atom to which they are attached form: 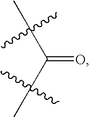
then A is alkyl; and
(g) when R¹ and R³ are —OH, R² is cycloalkylalkyl, and A and B are both H, then R^{4a} is other than —NH₂ or —OH;
or a pharmaceutically acceptable salt thereof.

2. The compound according to claim 1, wherein R¹ is —CON(R^{5d})(R^{6d}).

3. The compound according to claim 1, wherein R¹ is —OR^{5a} or —CH₂OR^{5e}.

4. The compound according to claim 1, wherein R² is cycloalkylalkyl.

5. The compound according to claim 1, wherein R² is alkyl.

6. The compound according to claim 1, wherein R³ is —OR^{5f} or —N(R^{5g})—C(═O)-Z.

7. The compound according to claim 1, wherein each Y is independently a single bond, —[C(R^{5h})(R^{6h})]ₜ—, or —C(═O)—.

8. The compound according to claim 1, wherein each Z is independently H, alkyl, heterocycloalkyl, aryl, or —WR⁷.

9. The compound according to claim 1, wherein R⁷ is —C(═O)—R^{5j}.

10. The compound according to claim 1, wherein R⁷ is —C(═O)—N(R^{5k})(R^{6k}).

11. The compound according to claim 1, wherein R⁸ is —N(R^{5k})(R^{6k}) and R^{5k} and R^{6k} together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂.

12. The compound according to claim 1, wherein A and B are both H.

13. The compound according to claim 1, wherein R^{4a} is —[C(R^{5m})(R^{6m})]ₛ—R^{4b} or —OR⁵ⁿ, or R^{4a} and B taken together with the carbon atom to which they are attached form:

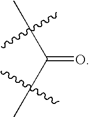

14. The compound according to claim 1, wherein R^{4a} is —[C(R^{5m})(R^{6m})]ₛ—R^{4b}.

15. The compound according to claim 1, wherein R^{4b} is alkenyl, aryl, heteroaryl, —N(R^{5p})—Y-Z, —C(═O)—R⁸, or —[C(R^{5q})(R^{6q})]ₛ—C(═O)—N(R^{5r})—WR⁷.

16. The compound according to claim 1, wherein t is 1 or 2.

17. The compound according to claim 1, wherein R¹ is —OR^{5a} and R² is cycloalkylalkyl.

18. The compound according to claim 1, wherein R¹ is —OR^{5a} and R³ is —OR^{5f} or —N(R^{5g})—C(═O)-Z.

19. The compound according to claim 1, wherein R² is cycloalkylalkyl and R³ is —OR^{5f} or —N(R^{5g})—C(═O)-Z.

20. The compound according to claim 1, wherein R¹ is —OR^{5a}, R²is cycloalkylalkyl, and R³ is —OR^{5f} or —N(R^{5g})—C(═O)-Z.

21. The compound according to claim 1, wherein R^{4a} and B taken together with the carbon atom to which they are attached form:

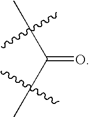

22. The compound according to claim 1, wherein each R^{5a}, R^{5b}, and R^{5d} is independently H or alkyl.

23. The compound according to claim 1, wherein each R^{5f}, R^{5g}, R^{5h}, R⁵ⁱ, R^{5j}, R^{5k}, R^{5m}, R⁵ⁿ, R^{5p}, R^{5q}, and R^{5r} is independently H, alkyl, or aryl.

24. The compound according to claim 1, wherein A is H, and R^{4a} and B taken together with the carbon atom to which they are attached form:

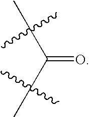

25. The compound according to claim 1, wherein R¹ is —OH and R² is cyclopropylmethyl.

26. The compound according to claim 1, wherein R^{4a} is —[C(R^{5m})(R^{6m})]ₛ—R^{4b} and R^{4b} is —[C(R^{5q})(R^{6q})]ₛ—C(═O)—N(R^{5r})—W—CO₂H or —N(R^{5p})—Y-Z, wherein Z is —W—C(═O)—OH, alkyl, cycloalkyl, cycloalkylalkyl, heterocycloalkyl, aryl, or heteroaryl, said alkyl, cycloalkyl, cycloalkylalkyl, heterocycloalkyl, aryl, or heteroaryl groups being substituted with —CO₂H, or R^{5p} and Z together with the atoms through which they are connected form a 4- to 8-membered heterocycloalkyl ring, said heterocycloalkyl ring being substituted with —CO₂H.

27. The compound according to claim 1, wherein R³ is —N(R^{5g})—Y-Z.

28. The compound according to claim 27, wherein said Z of said —N(R^{5g})—Y-Z is —W—C(═O)—OH or alkyl, cycloalkyl, cycloalkylalkyl, heterocycloalkyl, aryl, or heteroaryl, said alkyl, cycloalkyl, cycloalkylalkyl, heterocycloalkyl, aryl, or heteroaryl groups being substituted with —CO₂H.

29. The compound according to claim 25, of Formula Ia or IIb: 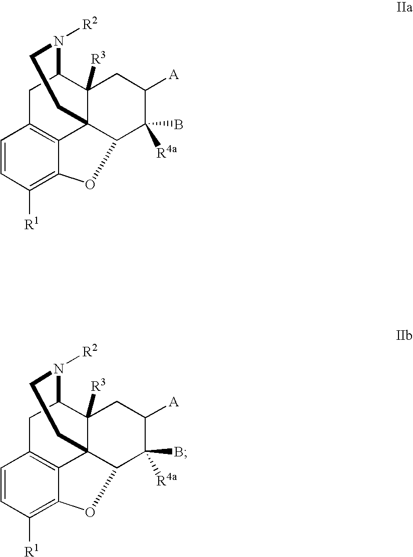
or a pharmaceutically acceptable salt thereof.

30. The compound according to claim 1 of Formula III: 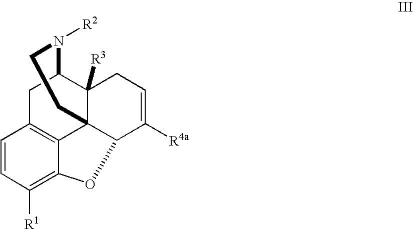
or a pharmaceutically acceptable salt thereof.

31. The compound according to claim 1 of Formula IVa or IVb: 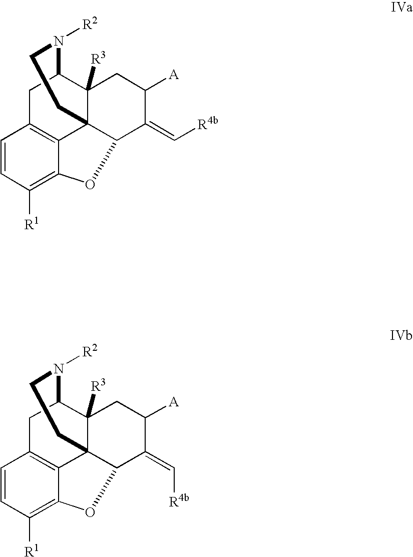
or a pharmaceutically acceptable salt thereof.

32. The compound according to claim 31, wherein R^{4b} is —C(═O)—R⁸ or —[C(R^{5q})(R^{6q})]ₛ—C(═O)—N(R^{5r})—WR⁷.

33. The compound according to claim 32, wherein R^{4b} is —C(═O)—N(R^{5k})(R^{6k}).

34. The compound according to claim 33, wherein R^{5k} and R^{6k} together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂.

35. The compound according to claim 34, wherein R^{5k} and R^{6k} together with the nitrogen atom to which they are attached form a piperidine ring, optionally substituted with —C(═O)—OH.

36. The compound according to claim 31, wherein R^{4b} is —[C(R^{5q})(R^{6q})]ₛ—C(═O)—N(R^{5r})—W—C(═O)—R⁸.

37. The compound according to claim 36, wherein R^{4b} is —[C(R^{5q})(R^{6q})]ₛ—C(═O)—N(H)—W—C(═O)—OH.

38. The compound according to claim 3, wherein R^{5a} is H.

39. The compound according to claim 4, wherein R² is cyclopropylmethyl.

40. The compound according to claim 6, wherein R³ is —N(R^{5g})—C(═O)-Z.

41. The compound according to claim 40, wherein R³ is —N(R^{5g})—C(═O)—W—C(═O)—R⁸.

42. The compound according to claim 41, wherein R³ is —N(H)—C(═O)—W—C(═O)—OH.

43. The compound according to claim 14, wherein R^{4b} is —N(R^{5p})—Y-Z.

44. The compound according to claim 43, wherein R^{4b} is —N(H)—W—C(═O)—R⁸.

45. The compound according to claim 44, wherein R^{4b} is —N(H)—W—C(═O)—OH.

46. The compound according to claim 9, wherein R^{5j} is H.

47. The compound according to claim 30, wherein R^{4a} is aryl or heteroaryl.

48. The compound according to claim 30, wherein R^{4a} is —C(═O)—R⁸.

49. The compound according to claim 48, wherein R⁸ is —OR^{5j} and R^{5j} is H or alkyl.

50. The compound according to claim 48, wherein R⁸ is —N(R^{5k})(R^{6k}).

51. The compound according to claim 50, wherein R^{6k} is alkyl or aralkyl, or R^{5k} and R^{6k} together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂.

52. The compound according to claim 30, wherein R^{4a} is —[C(R^{5q})(R^{6q})]ₛ—(═O)—N(R^{5r})—WR⁷.

53. The compound according to claim 52, wherein R^{4a} is —[C(R^{5q})(R^{6q})]ₛ—C(═O)—N(H)—[C(R⁵ⁱ)(R⁶ⁱ)]ₜ—R⁷, t is 1, 2, or 3, R⁷ is —C(═O)—OR^{5j}, and R^{5j} is H or alkyl.

54. The compound according to claim 12, wherein R^{4a} is —C(═O)—R⁸ or —[C(R^{5q})(R^{6q})]ₛ—C(═O)—N(R^{5r})—WR⁷.

55. The compound according to claim 54, wherein R^{4a} is —C(═O)—OR^{5j}.

56. The compound according to claim 54, wherein R^{4a} is —[C(R^{5q})(R^{6q})]—C(═O)—N(H)—CH₂R⁷.

57. The compound according to claim 31, wherein A is H.

58. The compound according to claim 57 of Formula IVa.

59. The compound according to claim 57, of Formula IVb.

60. The compound according to claim 57, wherein R^{4b} is alkyl, —C(═O)—R⁸, or —[C(R^{5q})(R^{6q})]ₛ—C(═O)—N(R^{5r})—WR⁷.

61. The compound according to claim 60, wherein R^{4b} is —CH₂OH.

62. The compound according to claim 60, wherein R^{4b} is —C(═O)—OR^{5j} and R^{5j} is H or alkyl.

63. The compound according to claim 60, wherein R^{4b} is —C(═O)—N(R^{5k})(R^{6k}), R^{5k} is H, and R^{6k} is alkyl, or R^{5k} and R^{6k} together with the nitrogen atom to which they are attached form a 4- to 8-membered heterocycloalkyl ring, the heterocycloalkyl ring optionally interrupted by one or more additional heteroatom moieties selected from nitrogen, oxygen, sulfur, S(═O), and S(═O)₂.

64. The compound according to claim 63, wherein R^{5k} and R^{6k} together with the nitrogen atom to which they are attached form a 4- to 8-membered heterocycloalkyl ring, the heterocycloalkyl ring optionally interrupted by one or more additional heteroatom moieties selected from nitrogen, oxygen, sulfur, S(═O), and S(═O)₂.

65. The compound according to claim 64, wherein the heterocycloalkyl ring is substituted with carboxy or alkoxycarbonyl.

66. The compound according to claim 60, wherein R^{4b} is —C(═O)—N(H)—[C(R⁵ⁱ)(R⁶ⁱ)]ₜ—C(═O)—R⁸, R⁸ is —OH or —O-alkyl, and t is 1, 2, or 3.

67. The compound according to claim 60, wherein R^{4b} is —C(═O)—OH.

68. The compound according to claim 66, wherein R³ is —N(R^{5g})—C(═O)-Z.

69. The compound according to claim 68, wherein Z is alkyl or aryl.

70. The compound according to claim 29, wherein A and B are both H and R^{4a} is —N(R^{5p})—Y-Z or —OR⁵ⁿ, wherein R⁵ⁿ is H, alkyl, cycloalkyl, cycloalkylalkyl, or aralkyl.

71. The compound according to claim 70, wherein R⁵ⁿ is H or alkyl.

72. The compound according to claim 70, wherein Y is a single bond or —[C(R^{5h})(R^{6h})]ₜ—.

73. The compound according to claim 70, wherein R³ is —N(R^{5g})—C(═O)—WR⁷.

74. The compound according to claim 73, wherein R⁷ is —C(═O)—OH.

75. The compound according to claim 70, wherein Z is alkyl, aryl, or —WR⁷.

76. The compound according to claim 70, wherein R^{4b} is —N(R^{5p})—Y-Z and R^{5p} and Z together with the atoms through which they are connected form a 4- to 8-membered heterocycloalkyl ring, said heterocycloalkyl ring optionally interrupted by one or more additional heteroatom moieties selected from nitrogen, oxygen, sulfur, S(═O), and S(═O)₂.

77. The compound according to claim 76, wherein the heterocycloalkyl ring is substituted with carboxy or alkoxycarbonyl.

78. The compound according to claim 29, wherein R³ is —N(R^{5g})—Y-Z.

79. The compound according to claim 78, wherein Y is —C(═O)—.

80. The compound according to claim 79, wherein Z is alkyl or —WR⁷.

81. The compound according to claim 80, wherein said alkyl is methyl.

82. The compound according to claim 29, of Formula Ia wherein R¹ is —OH, R² is cyclopropylmethyl, R³ is —NH—C(═O)—CH₃, A and B are H, and R^{4a} is —NH—[C(R⁵ⁱ)(R⁶ⁱ)]ₜ—COOH.

83. The compound according to claim 82, wherein R^{4a} is —NHCH₂COOH.

84. The compound according to claim 80, wherein Z is —WR⁷.

85. The compound according to claim 84, wherein WR⁷ is —[C(R⁵ⁱ)(R⁶ⁱ)]ₜ—COOH.

86. The compound according to claim 85, wherein R⁵ⁱ and R⁶ⁱ are each H.

87. The compound according to claim 86, wherein t is 3.

88. The compound according to claim 87, wherein R^{4a} is —OH and A and B are each H, or R^{4a} and B taken together with the carbon atom to which they are attached form: 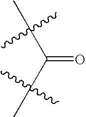
and A is H.

89. The compound according to claim 88, wherein R^{4a} and B taken together with the carbon atom to which they are attached form: 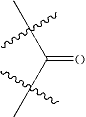
and A is H.

90. The compound according to claim 88, of Formula IIa wherein R^{4a} is —OH and A and B are each H.

91. The compound according to claim 88, of Formula IIb wherein R^{4a} is —OH and A and B are each H.

92. The compound according to claim 31, wherein R¹ and R³ are each —OH, R² is cyclopropylmethyl, and A is H.

93. The compound according to claim 92, wherein R^{4b} is C(═O)—N(R^{5k})(R^{6k}), wherein R^{5k} and R^{6k} together with the nitrogen atom to which they are attached form a 4- to 12-membered heterocycloalkyl ring, said heterocycloalkyl ring optionally interrupted by one or more additional heteroatom moieties selected from nitrogen, oxygen, sulfur, S(═O), and S(═O)₂.

94. The compound according to claim 93, wherein R^{4b} is:

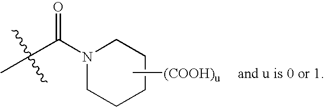

95. The compound according to claim 94 of Formula IVa, wherein R^{4b} is:

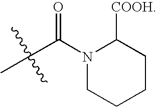

96. The compound according to claim 94 of Formula IVb, wherein R^{4b} is:

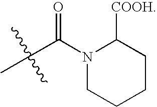

97. The compound according to claim 94 of Formula IVa, wherein R^{4b} is:

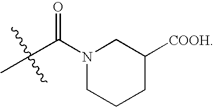

98. The compound according to claim 92, wherein R^{4b} is —[C(R^{5q})(R^{6q})]ₛ—C(═O)—N(R^{5r})—WR⁷.

99. The compound according to claim 98, wherein R^{4b} is —C(═O)—NH—[C(R⁵ⁱ)(R⁶ⁱ)]ₜ—COOH.

100. The compound according to claim 99, wherein R^{4b} is —C(═O)—NH—(CH₂)ₜ—COOH.

101. The compound according to claim 100 of Formula IVa, wherein t is 1.

102. The compound according to claim 100 of Formula IVb, wherein t is 1.

103. The compound according to claim 100 of Formula IVa, wherein t is 3.

104. The compound according to claim 1, selected from the group consisting of:
4,5α-Epoxy-6-carboxy-6,7-deshydro-3,14β-dihydroxy-17-(cyclopropylmethyl)-morphinan;
4,5α-Epoxy-6(α/β)-carboxy-3,14β-dihydroxy-17-(cyclopropylmethyl)morphinan;
4,5α-Epoxy-3,14β-dihydroxy-6,6Z(N-acryloylamino-acetic acid)-17-(cyclopropylmethyl)morphinan;
4,5α-Epoxy-3,14β-dihydroxy-6,6Z(N-acryloyl-piperidine-2(R/S)-carboxylic acid methyl ester)-17-(cyclopropylmethyl)morphinan;
4,5α-Epoxy-3,14β-dihydroxy-6,6Z(N-acryloyl-piperidine-3(R/S)-carboxylic acid)-17-(cyclopropylmethyl)morphinan;
4,5α-Epoxy-3,14β-dihydroxy-6,6E(carboxyethenyl)-17-(cyclopropylmethyl)morphinan;
4,5α-Epoxy-3,14β-dihydroxy-6,6E(N-acryloylamino-acetic acid)-17-(cyclopropylmethyl)morphinan;
4,5α-Epoxy-3,14β-dihydroxy-6,6E(N-acryloylamino-butyric acid)-17-(cyclopropylmethyl)morphinan;
4,5α-Epoxy-3,14β-dihydroxy-6,6E(N-acryloyl-piperidine-2(R/S)-carboxylic acid)-17-(cyclopropylmethyl)morphinan;
4,5α-Epoxy-3,14β-dihydroxy-6,6E(N-acryloyl-piperidine-3(R/S)-carboxylic acid)-17-(cyclopropylmethyl)morphinan;
4,5α-Epoxy-3,14β-dihydroxy-6,6E(N-acryloyl-piperidine-4-carboxylic acid)-17-(cyclopropylmethyl)morphinan;
4,5α-Epoxy-3-hydroxy-14β-acetylamino-6 β-carboxymethylamino-17-(cyclopropylmethyl)morphinan;
4,5α-Epoxy-3-hydroxy-14β-acetylamino-6 β-(4-carboxypiperidino)-17-(cyclopropylmethyl)morphinan;
4,5α-Epoxy-3-hydroxy-14β-acetylamino-6 β-(4-carboxybenzylamino)-17-(cyclopropylmethyl)morphinan;
4,5α-Epoxy-3-hydroxy-14β-[(4-carboxy)phenylcarbonylamino]-6-keto-17-(cyclopropylmethyl)morphinan;
4,5α-Epoxy-3-hydroxy-14β-[(4-carboxy)propionylamino]-6-keto-17-(cyclopropylmethyl)morphinan;
4,5α-Epoxy-3-hydroxy-14β-[(4-carboxy)propionylamino]-6-β-hydroxy-17-(cyclopropylmethyl)morphinan;
4,5α-Epoxy-3-hydroxy-14β-[(4-carboxy)propionylamino]-6-α-hydroxy-17-(cyclopropylmethyl)morphinan; and
stereoisomers, prodrugs, pharmaceutically acceptable salts, hydrates, solvates, acid hydrates, and N-oxides thereof.

105. The compound according to claim 1, selected from the group consisting of:
4,5α-Epoxy-3,14β-dihydroxy-6,6Z(N-acryloylamino-acetic acid)-17-(cyclopropylmethyl)morphinan;
4,5α-Epoxy-3,14β-dihydroxy-6,6Z(N-acryloyl-piperidine-2(R/S)-carboxylic acid methyl ester)-17-(cyclopropylmethyl)morphinan;
4,5α-Epoxy-3,14β-dihydroxy-6,6E(N-acryloylamino-acetic acid)-17-(cyclopropylmethyl)morphinan;
4,5α-Epoxy-3,14β-dihydroxy-6,6E(N-acryloylamino-butyric acid)-17-(cyclopropylmethyl)morphinan;
4,5α-Epoxy-3,14β-dihydroxy-6,6E(N-acryloyl-piperidine-2(R/S)-carboxylic acid)-17-(cyclopropylmethyl)morphinan;
4,5α-Epoxy-3,14β-dihydroxy-6,6E(N-acryloyl-piperidine-3(R/S)-carboxylic acid)-17-(cyclopropylmethyl)morphinan;
4,5α-Epoxy-3-hydroxy-14β-acetylamino-6 β-carboxymethylamino-17-(cyclopropylmethyl)morphinan;
4,5α-Epoxy-3-hydroxy-14β-[(4-carboxy)propionylamino]-6-keto-17-(cyclopropylmethyl)morphinan;
4,5α-Epoxy-3-hydroxy-14β-[(4-carboxy)propionylamino]-6-β-hydroxy-17-(cyclopropylmethyl)morphinan;
4,5α-Epoxy-3-hydroxy-14β-[(4-carboxy)propionylamino]-6-α-hydroxy-17-(cyclopropylmethyl)morphinan; and
stereoisomers, prodrugs, pharmaceutically acceptable salts, hydrates, solvates, acid hydrates, and N-oxides thereof.

106. The compound according to claim 1, selected from the group consisting of:
4,5α-Epoxy-3,14β-dihydroxy-6,6Z(N-acryloylamino-acetic acid)-17-(cyclopropylmethyl)morphinan;
4,5α-Epoxy-3,14β-dihydroxy-6,6E(N-acryloyl-piperidine-2(R/S)-carboxylic acid)-17-(cyclopropylmethyl)morphinan;
4,5α-Epoxy-3-hydroxy-14β-[(4-carboxy)propionylamino]-6-keto-17-(cyclopropylmethyl)morphinan;
4,5α-Epoxy-3-hydroxy-14β-[(4-carboxy)propionylamino]-6-β-hydroxy-17-(cyclopropylmethyl)morphinan;
4,5α-Epoxy-3-hydroxy-14β-[(4-carboxy)propionylamino]-6-α-hydroxy-17-(cyclopropylmethyl)morphinan; and
stereoisomers, prodrugs, pharmaceutically acceptable salts, hydrates, solvates, acid hydrates, and N-oxides thereof.

107. The compound according to claim 27, wherein R^{4a} is —[C(R^{5m})(R^{6m})]ₛ—R^{4b}, and R^{4b} is —[C(R^{5q})(R^{6q})]ₛ—C(═O)—N(R^{5r})—W—CO₂H or —N(R^{5p})—Y-Z, and said Z of said —N(R^{5p})—Y-Z is —W—C(═O)—OH, alkyl, cycloalkyl, cycloalkylalkyl, heterocycloalkyl, aryl or heteroaryl, said alkyl, cycloalkyl, cycloalkylalkyl, heterocycloalkyl, aryl or heteroaryl groups being substituted with —CO₂H, or R^{5p} and Z together with the atoms through which they are connected form a 4- to 8-membered heterocycloalkyl ring, said heterocycloalkyl ring being substituted with —CO₂H.

108. The compound according to claim 28, wherein R^{4a} is —[C(R^{5m})(R^{6m})]ₛ—N(R^{5p})—Y-Z.

109. A compound of Formula Ib: 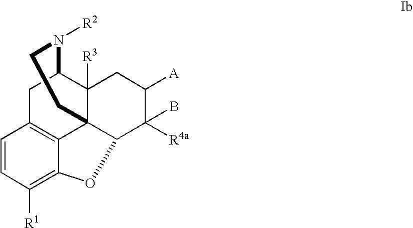
wherein:
R¹ is —OR^{5a}, —N(R^{5b})(R^{6b}), —COOR^{5c}, —CON(R^{5d})(R^{6d}), or —CH₂OR^{5e};
R² is H, alkyl, cycloalkyl, cycloalkylalkyl, aralkyl, aryl, or alkenyl;
R³ is —N(R^{5g})—Y-Z, wherein —N(R^{5g})—Y-Z is other than —NH₂;
each Y is independently a single bond, —[C(R^{5h})(R^{6h})]ₜ—, —C(═O)—, or —S(═O)₂—;
each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;
each W is independently —[C(R⁵ⁱ)(R⁶ⁱ)]ₜ—;
each R⁷ is independently —C(═O)—R⁸;
each R⁸ is independently —OR^{5j} or —N(R^{5k})(R^{6k});
A and B are each independently H or alkyl, or together represent a double bond between the carbon atoms to which they are attached;
R^{4a} is —[C(R^{5m})(R^{6m})]ₛ—R^{4b} or —OR⁵ⁿ, or R^{4a} and B taken together with the carbon atom to which they are attached may form: 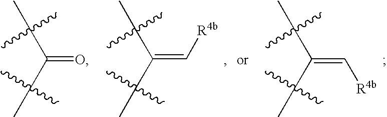
provided that when B is alkyl, then R^{4a} is —[C(R^{5m})(R^{6m})]ₛ—R^{4b};
R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;
each R^{5a}, R^{5b}, R^{5c}, R^{5d}, and R^{5e} is independently H, alkyl, cycloalkyl, cycloalkylalkyl, or aralkyl;
each R^{5g}, R^{5h}, R⁵ⁱ, R^{5j}, R^{5k}, R^{5m}, R⁵ⁿ, R^{5p}, R^{5q} and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;
each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
each s is independently 0 or 1; and
each t is independently an integer from 1 to 12;
provided that:
when R¹ is —OH or —O-alkyl, R² is H, cycloalkylalkyl, or alkenyl, R^{5g} is H, alkyl, cycloalkylalkyl, or aralkyl, Y is —C(═O)— or a single bond, A is H, and either B is H and R^{4a} is —OR⁵ⁿ or B and R^{4a} taken together with the carbon atom to which they are attached form: 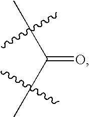
then Z is heterocycloalkyl, heteroaryl, or —WR⁷;
or a pharmaceutically acceptable salt thereof.

110. The compound according to claim 109, wherein R¹ is —CON(R^{5d})(R^{6d}).

111. The compound according to claim 110, wherein R^{5d} and R^{6d} are each H.

112. A compound of Formula Ic: 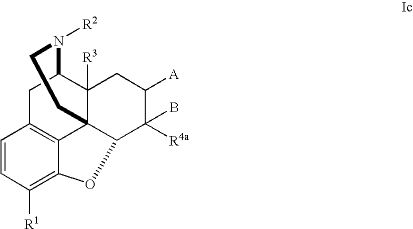
wherein:
R¹ is —OR^{5a}, —N(R^{5b})(R^{6b}), —COOR^{5c}, —CON(R^{5d})(R^{6d}), or CH₂OR^{5e};
R² is H, alkyl, cycloalkyl, cycloalkylalkyl, aralkyl, aryl, or alkenyl;
R³ is —OR^{5f} or —N(R^{5g})—Y-Z;
each Y is independently a single bond, —[C(R^{5h})(R^{6h})]ₜ—, —C(═O)—, or —S(═O)₂—;
each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;
each W is independently —[C(R⁵ⁱ)(R⁶ⁱ)]ₜ—;
each R⁷ is independently —C(═O)—R⁸;
each R⁸is independently —OR^{5j} or —N(R^{5k})(R^{6k});
A and B are each independently H or alkyl, or together represent a double bond between the carbon atoms to which they are attached;
R^{4a} is —[C(R^{5m})(R^{6m})]ₛ—R^{4b}, or
when A is H or alkyl, R^{4a} and B taken together with the carbon atom to which they are attached may form: 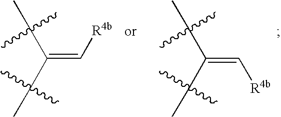
R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;
each R^{5a}, R^{5b}, R^{5c}, R^{5d}, and R^{5e} is independently H, alkyl, cycloalkyl, cycloalkylalkyl, or aralkyl;
each R^{5f}, R^{5g}, R^{5h}, R⁵ⁱ, R^{5j}, R^{5k}, R^{5m}, R^{5p}, R^{5q} and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;
each R^{6b}, R^{6d}, R^{6h}, R⁶ⁱ, R^{6k}, R^{6m}, and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d}, or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
each s is independently 0 or 1; and
each t is independently an integer from 1 to 12;
provided that:
(a) when R^{4a} is —N(R^{5p})-Z, at least one of R^{5p} and Z is other than H or haloalkyl;
(b) when R¹ and R³ are both —OH, R² is alkyl, cycloalkylalkyl, aryl, aralkyl, or alkenyl, A and B are each H, and R^{4a} is —NR^{5p}—C(═O)-Z, then R^{5p} is other than H or Z is other than alkyl;
(c) when R¹ and R³ are both —OH, R²is cyclopropylmethyl, and A and B together represent a double bond between the carbon atoms to which they are attached, then R^{4a} is other than phenyl, indolyl, or pyrrolyl;
(d) when R¹ and R³ are both —OH, R² is alkyl, cycloalkylalkyl, aryl, aralkyl, or alkenyl, A and B are each H, and R^{4a} is —NR^{5p}-Z, then at least one of R^{5p} and Z is other than haloalkyl; and
(e) when R¹ and R³ are —OH, R² is cycloalkylalkyl, and A and B are both H, then R^{4a} is other than —NH₂;
or a pharmaceutically acceptable salt thereof.

113. The compound according to claim 112, wherein R¹ is —CON(R^{5d})(R^{6d}).

114. The compound according to claim 113, wherein R^{5d} and R^{6d} are each H.

115. A compound of Formula Id: 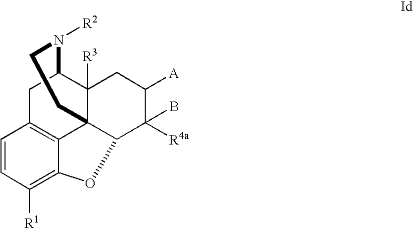
wherein:
R¹ is —OR^{5a}, —N(R^{5b})(R^{6b}), —COOR^{5c}, —CON(R^{5d})(R^{6d}), or —CH₂OR^{5e};
R² is H, alkyl, cycloalkyl, cycloalkylalkyl, aralkyl, aryl, or alkenyl;
R³is —OR^{5f} or —N(R^{5g})—Y-Z;
each Y is independently a single bond, —[C(R^{5h})(R^{6h})]ₜ—, —C(═O)—, or —S(═O)₂—;
each Z is independently H, alkyl, cycloalkyl, cycloalkylalkyl, heterocycloalkyl, aryl, aralkyl, aralkenyl, heteroaryl, or WR⁷, provided that when Y is —C(═O)— or —S(═O)₂—, then Z is other than H;
each W is independently —[C(R⁵ⁱ)(R⁶ⁱ)]ₜ—;
each R⁷ is independently —C(═O)—R⁸;
each R⁸ is independently —OR^{5j} or —N(R^{5k})(R^{6k});
A is H or alkyl and B is alkyl, or together A and B represent a double bond between the carbon atoms to which they are attached;
R^{4a} is —[C(R^{5m})(R^{6m})]ₛ—R^{4b}, or
when A is H or alkyl, R^{4a} and B taken together with the carbon atom to which they are attached may form: 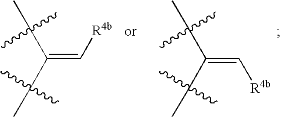
R^{4b} is alkenyl, alkynyl, alkyl, cycloalkyl, cycloalkylalkyl, aralkyl, aryl, heteroaryl, —N(R^{5p})—Y-Z, —C(═O)—R⁸, or —[C(R^{5q})(R^{6q})]ₛ—C(═O)—N(R^{5r})—WR⁷;
each R^{5a}, R^{5b}, R^{5c}, R^{5d} and R^{5e} is independently H, alkyl, cycloalkyl, cycloalkylalkyl, or aralkyl;
each R^{5f}, R^{5g}, R^{5h}, R⁵ⁱ, R^{5j}, R^{5k}, R^{5m}, R⁵ⁿ, R^{5p}, R^{5q} and R^{5r} is independently H, alkyl, cycloalkyl, cycloalkylalkyl, aryl, heteroaryl, or aralkyl; or when R^{4b} is —N(R^{5p})—Y-Z, R^{5p} and Z together with the atoms through which they are connected may form a 4- to 8-membered heterocycloalkyl ring, said heterocycloalkyl ring optionally interrupted by one or more additional heteroatom moieties selected from nitrogen, oxygen, sulfur, S(═O), and S(═O)₂;
each R^{6b}, R^{6d}, R^{6h}, R⁶ⁱ, R^{6k}, R^{6m} and R^{6q} is independently H, alkyl, aralkyl, or aryl, or when R¹ is —N(R^{5b})(R^{6b}) or —CON(R^{5d})(R^{6d}), or R⁸ is —N(R^{5k})(R^{6k}), then R^{5b} and R^{6b}, R^{5d} and R^{6d} or R^{5k} and R^{6k}, together with the nitrogen atom to which they are attached may form a 4- to 12-membered heterocycloalkyl ring, said heterocycloalkyl ring optionally interrupted by one or more additional heteroatom moieties selected from nitrogen, oxygen, sulfur, S(═O), and S(═O)₂;
each s is independently the integer 0 or 1; and
each t is independently an integer from 1 to 12;
provided that:
when R¹ and R³ are both —OH, R² is cyclopropylmethyl and A and B together represent a double bond between the carbon atoms to which they are attached, then R^{4a} is other than phenyl, indolyl, or pyrrolyl;
or a pharmaceutically acceptable salt thereof.

116. The compound according to claim 115, wherein R¹ is —CON(R^{5d})(R^{6d}).

117. The compound according to claim 116, wherein R^{5d} and R^{6d} are each H.

118. A pharmaceutical composition, comprising:
a pharmaceutically acceptable carrier; and
a compound according to claim 1.

119. The pharmaceutical composition according to claim 118,
further comprising at least one opioid.

120. The pharmaceutical composition according to claim 119,
wherein the opioid is alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol, or mixtures thereof.

121. A method of preventing or treating a condition or disease associated with binding opioid receptors in a patient in need thereof, comprising the step of:
administering to said patient a composition comprising an effective amount of a compound according to claim 1.

122. The method according to claim 121,
wherein the binding antagonizes the activity of the opioid receptors.

123. The method according to claim 122,
wherein the condition or disease is pain, gastrointestinal dysfunction, or ileus.

124. The method of claim 123, wherein the ileus is post-operative ileus.

125. A method according to claim 123,
wherein the condition is pain and the composition further comprises an effective amount of an opioid.

126. The method according to claim 122,
wherein the compound binds t opioid receptors.

127. The method according to claim 126,
wherein the μ opioid receptors are located in the central nervous system.

128. The method according to claim 126,
wherein the μ opioid receptors are located peripherally to the central nervous system.

129. The method according to claim 122,
wherein the compound does not substantially cross the blood-brain barrier.

130. The method of treating or preventing a side effect associated with an opioid, comprising the step of:
administering to a patient in need thereof, a composition comprising an effective amount of a compound according to claim 1.

131. The method according to claim 130,
wherein the opioid is endogenous.

132. The method according to claim 130,
wherein the opioid is exogenous.

133. The method according to claim 130, wherein the composition further comprises an effective amount of at least one opioid.

134. The method according to claim 130,
wherein the side effect is selected from the group consisting of constipation, opioid-induced bowel dysfunction, nausea, vomiting, and combinations thereof.

135. The method according to claim 130,
wherein the administering step occurs before, during or after a step of administering at least one opioid.

136. The method according to claim 133,
wherein the opioid is alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol, or mixtures thereof.
